(19) United States
(12) Patent Application Publication
(10) Pub. No.: US 2025/0352520 A1
(43) Pub. Date: Nov. 20, 2025

(54) USE OF DEXPRAMIPEXOLE FOR THE TREATMENT OF EOSINOPHILIC COPD

(71) Applicant: Areteia Therapeutics, Inc., Chapel Hill, NC (US)

(72) Inventors: Eric BRADFORD, Chapel Hill, NC (US); Calman PRUSSIN, Rockville, MD (US); Jonathan STEINFELD, Merion Station, PA (US); Steven W YANCEY, Apex, NC (US); Stephanie HARRIS, Raleigh, NC (US)

(73) Assignee: Areteia Therapeutics, Inc., Chapel Hill, NC (US)

(21) Appl. No.: 19/211,099

(22) Filed: May 16, 2025

Related U.S. Application Data
(60) Provisional application No. 63/648,963, filed on May 17, 2024.

(51) Int. Cl. A61K 31/428; A61P 11/00

(52) U.S. Cl. CPC A61K 31/428; A61P 11/00

(57) ABSTRACT

The present disclosure provides a method of treating eosinophilic COPD and/or eosinophilic inflammatory responses of eosinophilic COPD in a subject in need thereof. The method comprises orally administering to the subject a therapeutically effective amount of dexpramipexole, or a pharmaceutically acceptable salt thereof. The treating eosinophilic COPD can comprise reducing the rate of COPD exacerbation, reducing a mucus plug score, reducing the rate of hospitalizations due to exacerbations, reducing the rate of oral/parenteral steroid and/or antibiotic use for treatment of COPD, changing the level of one or more biomarkers associated with eosinophilic chronic obstructive pulmonary disorder (COPD), lung function decline, or any combination thereof.

### CROSS REFERENCE TO RELATED APPLICATIONS

This application claims the benefit of U.S. Provisional Application No. 63/648,963, filed on May 17, 2024. The disclosure of U.S. Provisional Application No. 63/648,963 is incorporated herein by reference.

### FIELD OF THE DISCLOSURE

The present disclosure relates to methods of treating chronic obstructive pulmonary disorder (COPD) in a human subject comprising orally administering to the subject a therapeutically effective amount of dexpramipexole, or a pharmaceutically acceptable salt thereof.

### BACKGROUND OF THE DISCLOSURE

COPD is a chronic progressive disease caused by chronic inflammation and destruction of the airways and lung parenchyma, and is usually associated with tobacco smoking or prolonged exposure to noxious particles and gases. COPD is the third leading cause of death worldwide, and affects 11.7 million people in America to date. The burden and prevalence of COPD are projected to increase due to both the continued exposure to COPD risk factors and the aging population.

COPD is characterized by chronic respiratory symptoms (e.g., dyspnea, cough, sputum production and/or flareups/exacerbations) due to abnormalities of the airways (bronchitis, bronchiolitis) and/or alveoli (emphysema) that cause persistent, often leading to progressive airflow obstruction. In addition to the daily symptom burden, poor quality of life and reduced health status, COPD is a progressive disease that gets worse over time, reflected by increases in exacerbations and by reductions in spirometry measures, such as forced expiratory volume in 1 second (e.g., FEV₁, FVC, FEV₁/FVC), over time. The diagnosis of COPD is confirmed by the patient's′ medical history and by the presence of non-fully reversible airflow limitation (i.e., post-bronchodilation FEV₁/forced vital capacity FVC<0.7).

The primary recommendation for treatment and prevention of COPD is smoking cessation. Drug therapy is useful to reduce symptoms, reduce the severity and frequency of exacerbations, and to improve QoL and health status. Although there are many medications approved for the treatment of COPD (e.g., short- and long-acting bronchodilators including β2-agonists and anticholinergics/anti-muscarinics and short- and long-acting inhaled glucocorticoids), 30 to 40% of patients continue to experience symptoms as well as moderate or severe exacerbations despite receiving inhaled triple therapy (i.e., inhaled corticosteroid, long-acting β2-agonist, and long-acting muscarinic antagonist).

Eosinophilic airway inflammation, which is most commonly encountered in asthma, has been also described in patients with COPD. Increased numbers of airway and circulating eosinophils are associated with an increased frequency of exacerbations, a high symptom burden, and impaired lung function. Conversely, maintaining lowered eosinophils has been linked with fewer exacerbations and hospital admissions in asthma.

Several drugs have demonstrated lowering eosinophil levels in asthma. For example, benralizumab, an anti-interleukin-5 receptor monoclonal antibody, effectively depleted eosinophils, decreased exacerbations, and improved lung function in patients with severe, uncontrolled, eosinophilic asthma in randomized, placebo-controlled phase 3 trials, CALIMA and SIROCCO. Similarly, in a multicenter, randomized, double-blind, double-dummy, phase 3, placebo controlled trial (MENSA), mepolizumab, a humanized monoclonal antibody against interleukin-5, significantly reduced asthma exacerbations and was associated with improvements in biomarkers of asthma control, including eosinophil counts.

Because of the success of eosinophil-lowering drugs benralizumab and mepolizumab in asthma, they were tested in patients with COPD with elevated eosinophils. However, these drugs did not demonstrate consistent or comparable clinical benefits as proven in asthma, even though an eosinophil-lowering effect was observed. For example, two phase 3, randomized, double-blind, placebo-controlled, parallel-group benralizumab trials (GALATHEA and TERRANOVA) were disappointing and failed to show statistically significant reduction of exacerbations in patients with COPD, even though all benralizumab doses led to substantial depletion of blood eosinophils throughout the duration of the trial. Similarly, mepolizumab was evaluated in multiple clinical studies for treatment of COPD (METREO, METREX), but also disappointing. While mepolizumab effectively reduced blood eosinophil counts, this effect was not associated with consistent improvements in lung function and clinical outcomes. Taken together, the results with eosinophil-lowering agents benralizumab and mepolizumab demonstrate that despite reported eosinophil involvement in COPD, eosinophil reduction or depletion may not be expected to ameliorate exacerbation outcomes for patients with COPD.

Accordingly, there is a high unmet need for novel therapies that deliver clinically relevant outcomes in the treatment and/or prevention of eosinophilic COPD.

Dexpramipexole ((6R)-2-amino-4,5,6,7-tetrahydro-6-(propylamino)benzothiazole) has a targeted effect on eosinophil hematopoiesis in the bone marrow by inhibiting the maturation of eosinophil precursor cells. Dexpramipexole has the following structure:

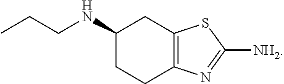

Dexpramipexole was initially identified for its neuroprotective activity in vitro and consequently its initial clinical development was focused on the treatment of amyotrophic lateral sclerosis (ALS). The safety and efficacy of dexpramipexole was evaluated in a Phase III pivotal study (233AS302; EMPOWER) in participants with ALS, which failed to demonstrate efficacy. However, a substantial, durable, and targeted reduction in absolute eosinophil count (AEC) was observed. Subsequently, the clinical development of dexpramipexole pivoted to the treatment of eosinophilic asthma and other eosinophilic associated disorders (EADs).

In a Phase II study in moderate to severe asthma (EXHALE-1), administration of a 150 mg BID dose of dexpramipexole dihydrochloride (equivalent to 112 mg of dexpramipexole) resulted in significant, dose-dependent, and durable reductions of eosinophils in blood and airways of up to approximately 80% (FIG. 1). In addition, clinically meaningful, placebo-corrected, change from baseline in pre-bronchodilator (pre-BD) forced expiratory volume in 1 second (FEV₁) were observed across the dose groups at various time-points.

It has now been unexpectedly discovered that dexpramipexole, or a pharmaceutically acceptable salt thereof, can be used to treat eosinophilic COPD and/or eosinophilic inflammatory responses of eosinophilic COPD.

### BRIEF SUMMARY OF THE DISCLOSURE

Provided herein are methods of reducing the rate of COPD exacerbations in a human subject with eosinophilic chronic obstructive pulmonary disorder (COPD), comprising orally administering to the subject a therapeutically effective amount of dexpramipexole, or a pharmaceutically acceptable salt thereof.

In some embodiments, dexpramipexole, or a pharmaceutically acceptable salt thereof, is administered in a daily dose of about 150 mg to about 300 mg per day. In some embodiments, dexpramipexole, or a pharmaceutically acceptable salt thereof, is administered in a daily dose of about 300 mg per day. In some embodiments, dexpramipexole, or a pharmaceutically acceptable salt thereof, is administered as about 300 mg once per day. In some embodiments, dexpramipexole, or a pharmaceutically acceptable salt thereof, is administered as about 150 mg twice daily. In some embodiments, dexpramipexole, or a pharmaceutically acceptable salt thereof, is administered in a daily dose of about 150 mg per day. In some embodiments, dexpramipexole, or a pharmaceutically acceptable salt thereof, is administered as about 150 mg once per day. In some embodiments, dexpramipexole, or a pharmaceutically acceptable salt thereof, is administered as about 75 mg twice daily.

In some embodiments, the subject has an elevated blood absolute eosinophil count (AEC). AEC can be expressed in units of cells/uL or ×10⁹/L. In some embodiments, the subject has a blood absolute eosinophil count (AEC) of ≥0.15×10⁹/L (150 cells/μL) before administration of dexpramipexole, or a pharmaceutically acceptable salt thereof. In some embodiments, the subject has a blood absolute eosinophil count (AEC) of ≥0.30×10⁹/L (300 cells/μL) before administration of dexpramipexole, or a pharmaceutically acceptable salt thereof.

In some embodiments, the subject has evidence of mucus plugs (i.e., a mucus plug score of ≥1) before administration of dexpramipexole, or a pharmaceutically acceptable salt thereof. In some embodiments, administering dexpramipexole, or a pharmaceutically acceptable salt thereof, reduces the presence of mucus plugs as demonstrated by reducing the mucus plug score.

In some embodiments, administering dexpramipexole, or a pharmaceutically acceptable salt thereof, improves the subject's mean forced expiratory volume in 1 second (FEV₁). In some embodiments, the subject's FEV₁ is increased by about 5% to about 50%. In some embodiments, the subject's FEV₁ is increased by about 5% to about 10%. In some embodiments, the subject's FEV₁ is increased by about 10% to about 30%. In some embodiments, the subject's FEV₁ is increased by about 16% to about 30%. In some embodiments, the subject's FEV₁ is increased by about 18% to about 30%. In some embodiments, the subject's FEV₁ is increased by about 20% to about 30%. In some embodiments, the subject's FEV₁ is increased by about 22% to about 30%. In some embodiments, the subject's FEV₁ is increased by about 24% to about 30%. In some embodiments, the subject's FEV₁ is increased by about 26% to about 30%. In some embodiments, the subject's FEV₁ is increased by about 28% to about 30%. In some embodiments, the subject's FEV₁ is increased by about 30% to about 50%. In some embodiments, the subject's FEV₁ is increased by about 30% to about 75%. In some embodiments, the subject's FEV₁ is increased by about 35% to about 75%. In some embodiments, the subject's FEV₁ is increased by about 40% to about 75%. In some embodiments, the subject's FEV₁ is increased by about 45% to about 75%. In some embodiments, the subject's FEV₁ is increased by about 55% to about 75%. In some embodiments, the subject's FEV₁ is increased by about 65% to about 75%. In some embodiments FEV₁ is measured pre-bronchodilation. In some embodiments FEV₁ is measured post-bronchodilation.

In some embodiments, the subject's FEV₁ is increased by about 30 to about 1000 ml. In some embodiments, the subject's FEV₁ is increased by about 40 to about 500 ml after administration. In some embodiments, the subject's FEV₁ is increased by about 200 mL to about 500 mL after administration. In some embodiments, the subject's FEV₁ is increased by about 250 mL to about 400 mL after administration. In some embodiments, the subject's FEV₁ is increased by about 300 mL to about 400 mL after administration. In some embodiments, the subject's FEV₁ is increased by about 350 mL to about 400 mL after administration. In some embodiments, the subject's FEV₁ is increased by about 50 to about 150 ml after administration. In some embodiments, the subject's FEV₁ is increased by about 100 ml after administration. In some embodiments, the subject's FEV₁ is increased by about 500 mL to about 900 mL. In some embodiments, the subject's FEV₁ is increased by about 550 mL to about 900 mL. In some embodiments, the subject's FEV₁ is increased by about 600 mL to about 900 mL. In some embodiments, the subject's FEV₁ is increased by about 650 mL to about 900 mL. In some embodiments, the subject's FEV₁ is increased by about 700 mL to about 900 mL. In some embodiments, the subject's FEV₁ is increased by about 750 mL to about 900 mL. In some embodiments, the subject's FEV₁ is increased by about 800 mL to about 900 mL. In some embodiments, the subject's FEV₁ is increased by about 850 mL to about 900 mL. In some embodiments FEV₁ is measured pre-bronchodilation. In some embodiments FEV₁ is measured post-bronchodilation.

In some embodiments, administering dexpramipexole, or a pharmaceutically acceptable salt thereof, improves the subject's forced vital capacity (FVC). In some embodiments, the subject's FVC is increased by about 5% to about 50%. In some embodiments, the subject's FVC is increased by about 5% to about 10%. In some embodiments, the subject's FVC is increased by about 10% to about 30%. In some embodiments, the subject's FVC is increased by about 30% to about 50%. In some embodiments, the subject's FVC is increased by about 6% to about 20%. In some embodiments, the subject's FVC is increased by about 8% to about 20%. In some embodiments, the subject's FVC is increased by about 10% to about 20%. In some embodiments, the subject's FVC is increased by about 12% to about 20%. In some embodiments, the subject's FVC is increased by about 14% to about 20%. In some embodiments, the subject's FVC is increased by about 16% to about 20%. In some embodiments, the subject's FVC is increased by about 18% to about 20%. In some embodiments, the subject's FVC is increased by about 10% to about 40%. In some embodiments, the subject's FVC is increased by about 15% to about 40%. In some embodiments, the subject's FVC is increased by about 20% to about 40%. In some embodiments, the subject's FVC is increased by about 25% to about 40%. In some embodiments, the subject's FVC is increased by about 30% to about 40%. In some embodiments, the subject's FVC is increased by about 35% to about 40%. In some embodiments FVC is measured pre-bronchodilation. In some embodiments FVC is measured post-bronchodilation.

In some embodiments, the subject's FVC is increased by about 30 ml to about 1000 ml after administration. In some embodiments, the subject's FVC is increased by about 40 to about 500 ml after administration. In some embodiments, the subject's FVC is increased by about 50 to about 150 ml after administration. In some embodiments, the subject's FVC is increased by about 100 ml after administration. In some embodiments, the subject's FVC is increased by about 200 mL to about 500 mL after administration. In some embodiments, the subject's FVC is increased by about 250 mL to about 500 mL after administration. In some embodiments, the subject's FVC is increased by about 300 mL to about 500 mL after administration. In some embodiments, the subject's FVC is increased by about 350 mL to about 500 mL after administration. In some embodiments, the subject's FVC is increased by about 400 mL to about 500 mL after administration. In some embodiments, the subject's FVC is increased by about 450 mL to about 500 mL after administration. In some embodiments, the subject's FVC is increased by about 500 mL to about 900 mL. In some embodiments, the subject's FVC is increased by about 550 mL to about 900 mL. In some embodiments, the subject's FVC is increased by about 600 mL to about 900 mL. In some embodiments, the subject's FVC is increased by about 650 mL to about 900 mL. In some embodiments, the subject's FVC is increased by about 700 mL to about 900 mL. In some embodiments, the subject's FVC is increased by about 750 mL to about 900 mL. In some embodiments, the subject's FVC is increased by about 800 mL to about 900 mL. In some embodiments, the subject's FVC is increased by about 850 mL to about 900 mL. In some embodiments FVC is measured pre-bronchodilation. In some embodiments FVC is measured post-bronchodilation.

In some embodiments, administering dexpramipexole, or a pharmaceutically acceptable salt thereof, improves the subject's FEV₁/FVC. In some embodiments, the subject's FEV₁/FVC is increased by about 0.1 or greater. In some embodiments, the subject's FEV₁/FVC is increased by about 0.2 or greater. In some embodiments FEV₁/FVC is measured pre-bronchodilation. In some embodiments FEV₁/FVC is measured post-bronchodilation.

In some embodiments, administering dexpramipexole, or a pharmaceutically acceptable salt thereof, improves the subject's forced mid-expiratory flow (FEF₂₅₋₇₅). In some embodiments FEF₂₅₋₇₅ is measured pre-bronchodilation. In some embodiments FEF₂₅₋₇₅ is measured post-bronchodilation.

In some embodiments, administering dexpramipexole, or a pharmaceutically acceptable salt thereof, improves the subject's ratio of forced expiratory volume in 3 seconds to FVC (FEV₃/FVC). In some embodiments FEV₃/FVC is measured pre-bronchodilation. In some embodiments FEV₃/FVC is measured post-bronchodilation.

In some embodiments, administering dexpramipexole, or a pharmaceutically acceptable salt thereof, improves a measurement selected from the group consisting of St. George's Respiratory Questionnaire for COPD Patients (SGRQ-C) (Meguro, M., et al., Chest 2006; 132: 456-463), COPD Assessment Test (CAT) (Jones, P. W., et al., Eur. Respir. J. 2009: 34: 648-654), Modified Medical Research Council Grading System (mMRC) (Bestall, J. C., et al., Thorax. 1999, 54 (7): 581-586), Evaluating Respiratory Symptoms in Chronic Obstructive Pulmonary Disease (E-RS:COPD) (Kline Leidy, N., et al., Chronic Obstr. Pulm. Dis. 2022 Oct. 26; 9(4):576-590), and any combination thereof.

In some embodiments, administering dexpramipexole, or a pharmaceutically acceptable salt thereof, improves the subject's SGRQ-C total score measurement. In some embodiments the SGRQ-C measurement improves by about 1 to about 40 points. In some embodiments the SGRQ-C measurement improves by about 10 to about 15 points. In some embodiments the SGRQ-C measurement improves by about 10 to about 20 points. In some embodiments the SGRQ-C measurement improves by about 20 to about 30 points. In some embodiments the SGRQ-C measurement improves by about 20 to about 40 points. In some embodiments the SGRQ-C measurement improves by about 5 to about 10 points. In some embodiments the SGRQ-C measurement improves by about 10 to about 15 points. In some embodiments the SGRQ-C measurement improves by about 15 to about 20 points.

In some embodiments, administering dexpramipexole, or a pharmaceutically acceptable salt thereof, improves the subject's CAT total score measurement. In some embodiments the CAT measurement improves by about 1 to about 30 points. In some embodiments the CAT measurement improves by about 10 to about 15 points. In some embodiments the CAT measurement improves by about 10 to about 20 points. In some embodiments the CAT measurement improves by about 20 to about 30 points. In some embodiments the CAT measurement improves by about 5 to about 10 points. In some embodiments the CAT measurement improves by about 10 to about 15 points. In some embodiments the CAT measurement improves by about 15 to about 20 points.

In some embodiments, prior to treatment with dexpramipexole or a pharmaceutically acceptable salt thereof, the subject is already receiving a treatment regimen comprising administration of an inhaled corticosteroid (ICS), thereby treating the COPD in the subject. In some embodiments, the treatment regimen comprises administration of an ICS and a long-acting β2 agonist (LABA). In some embodiments, the treatment regimen comprises administration of an ICS and a long-acting muscarinic antagonist (LAMA). In some embodiments, the treatment regimen comprises administration of an ICS, LABA, and LAMA. In some embodiments, the subject has received the treatment regimen for at least 12 weeks. In some embodiments, the subject has received a stable dose of the treatment regimen for at least 4 weeks. In some embodiments, the ICS is selected from the group consisting of beclomethasone, fluticasone, ciclesonide, mometasone, budesonide, flunisolide, and any combination thereof. In some embodiments, the LABA is selected from the group consisting of formoterol fumarate, salmeterol, salmeterol xinafoate, arformoterol tartrate, olodaterol, vilanterol, indacaterol, and any combination thereof. In some embodiments, the LAMA is selected from the group consisting of aclidinium, glycopyrrolate, glycopyrronium, tiotropium, umeclidinium, and any combination thereof. In some embodiments, prior to treatment with dexpramipexole or a pharmaceutically acceptable salt thereof, the subject is receiving one or more COPD medications selected from the group consisting of short-acting bronchodilators, long-acting bronchodilators, ultra-long-acting bronchodilators, xanthines, short-acting muscarinic antagonists, long-acting muscarinic antagonists, leukotriene antagonists, 5-lipoxygenase inhibitors, and any combination thereof.

In some embodiments, the subject is a current or former smoker. In some embodiments, the subject's COPD is from toxic exposure.

Further provided herein are methods of reducing the presence of mucus plugs as demonstrated by reducing a mucus plug score in a human subject with eosinophilic chronic obstructive pulmonary disorder (COPD), comprising orally administering to the subject a therapeutically effective amount of dexpramipexole, or a pharmaceutically acceptable salt thereof.

In some embodiments, dexpramipexole, or a pharmaceutically acceptable salt thereof, is administered in a daily dose of about 150 mg to about 300 mg per day. In some embodiments, dexpramipexole, or a pharmaceutically acceptable salt thereof, is administered in a daily dose of about 300 mg per day. In some embodiments, dexpramipexole, or a pharmaceutically acceptable salt thereof, is administered as about 300 mg once per day. In some embodiments, dexpramipexole, or a pharmaceutically acceptable salt thereof, is administered as about 150 mg twice daily. In some embodiments, dexpramipexole, or a pharmaceutically acceptable salt thereof, is administered in a daily dose of about 150 mg per day. In some embodiments, dexpramipexole, or a pharmaceutically acceptable salt thereof, is administered as about 150 mg once per day. In some embodiments, dexpramipexole, or a pharmaceutically acceptable salt thereof, is administered as about 75 mg twice daily.

In some embodiments, the subject has a blood absolute eosinophil count (AEC) of ≥0.15×10⁹/L before administration of dexpramipexole, or a pharmaceutically acceptable salt thereof. In some embodiments, the subject has a blood absolute eosinophil count (AEC) of ≥0.30×10⁹/L before administration of dexpramipexole, or a pharmaceutically acceptable salt thereof.

In some embodiments, the subject has evidence of mucus plugs (i.e., a mucus plug score of ≥1) before administration of dexpramipexole, or a pharmaceutically acceptable salt thereof. In some embodiments, administering dexpramipexole, or a pharmaceutically acceptable salt thereof, reduces the presence of mucus plugs as demonstrated by reducing the mucus plug score.

In some embodiments, administering dexpramipexole, or a pharmaceutically acceptable salt thereof, improves the subject's mean forced expiratory volume in 1 second (FEV₁). In some embodiments, the subject's FEV₁ is increased by about 5% to about 50%. In some embodiments, the subject's FEV₁ is increased by about 5% to about 10%. In some embodiments, the subject's FEV₁ is increased by about 10% to about 30%. In some embodiments, the subject's FEV₁ is increased by about 30% to about 50%. In some embodiments, the subject's FEV₁ is increased by about 16% to about 30%. In some embodiments, the subject's FEV₁ is increased by about 18% to about 30%. In some embodiments, the subject's FEV₁ is increased by about 20% to about 30%. In some embodiments, the subject's FEV₁ is increased by about 22% to about 30%. In some embodiments, the subject's FEV₁ is increased by about 24% to about 30%. In some embodiments, the subject's FEV₁ is increased by about 26% to about 30%. In some embodiments, the subject's FEV₁ is increased by about 28% to about 30%. In some embodiments, the subject's FEV₁ is increased by about 30% to about 75%. In some embodiments, the subject's FEV₁ is increased by about 35% to about 75%. In some embodiments, the subject's FEV₁ is increased by about 40% to about 75%. In some embodiments, the subject's FEV₁ is increased by about 45% to about 75%. In some embodiments, the subject's FEV₁ is increased by about 55% to about 75%. In some embodiments, the subject's FEV₁ is increased by about 65% to about 75%. In some embodiments FEV₁ is measured pre-bronchodilation. In some embodiments FEV₁ is measured post-bronchodilation.

In some embodiments, the subject's FEV₁ is increased by about 30 to about 1000 ml. In some embodiments, the subject's FEV₁ is increased by about 40 to about 500 ml after administration. In some embodiments, the subject's FEV₁ is increased by about 50 to about 150 ml after administration. In some embodiments, the subject's FEV₁ is increased by about 100 ml after administration. In some embodiments, the subject's FEV₁ is increased by about 200 mL to about 500 mL after administration. In some embodiments, the subject's FEV₁ is increased by about 250 mL to about 500 mL after administration. In some embodiments, the subject's FEV₁ is increased by about 300 mL to about 500 mL after administration. In some embodiments, the subject's FEV₁ is increased by about 350 mL to about 500 mL after administration. In some embodiments, the subject's FEV₁ is increased by about 400 mL to about 500 mL after administration. In some embodiments, the subject's FEV₁ is increased by about 450 mL to about 500 mL after administration. In some embodiments, the subject's FEV₁ is increased by about 500 mL to about 900 mL. In some embodiments, the subject's FEV₁ is increased by about 550 mL to about 900 mL. In some embodiments, the subject's FEV₁ is increased by about 600 mL to about 900 mL. In some embodiments, the subject's FEV₁ is increased by about 650 mL to about 900 mL. In some embodiments, the subject's FEV₁ is increased by about 700 mL to about 900 mL. In some embodiments, the subject's FEV₁ is increased by about 750 mL to about 900 mL. In some embodiments, the subject's FEV₁ is increased by about 800 mL to about 900 mL. In some embodiments, the subject's FEV₁ is increased by about 850 mL to about 900 mL. In some embodiments FEV₁ is measured pre-bronchodilation. In some embodiments FEV₁ is measured post-bronchodilation.

In some embodiments, administering dexpramipexole, or a pharmaceutically acceptable salt thereof, improves the subject's forced vital capacity (FVC). In some embodiments, the subject's FVC is increased by about 5% to about 50%. In some embodiments, the subject's FVC is increased by about 5% to about 10%. In some embodiments, the subject's FVC is increased by about 10% to about 30%. In some embodiments, the subject's FVC is increased by about 30% to about 50%. In some embodiments, the subject's FVC is increased by about 6% to about 20%. In some embodiments, the subject's FVC is increased by about 8% to about 20%. In some embodiments, the subject's FVC is increased by about 10% to about 20%. In some embodiments, the subject's FVC is increased by about 12% to about 20%. In some embodiments, the subject's FVC is increased by about 14% to about 20%. In some embodiments, the subject's FVC is increased by about 16% to about 20%. In some embodiments, the subject's FVC is increased by about 18% to about 20%. In some embodiments, the subject's FVC is increased by about 10% to about 40%. In some embodiments, the subject's FVC is increased by about 15% to about 40%. In some embodiments, the subject's FVC is increased by about 20% to about 40%. In some embodiments, the subject's FVC is increased by about 25% to about 40%. In some embodiments, the subject's FVC is increased by about 30% to about 40%. In some embodiments, the subject's FVC is increased by about 35% to about 40%. In some embodiments FVC is measured pre-bronchodilation. In some embodiments FVC is measured post-bronchodilation.

In some embodiments, the subject's FVC is increased by about 30 ml to about 1000 ml after administration. In some embodiments, the subject's FVC is increased by about 40 to about 500 ml after administration. In some embodiments, the subject's FVC is increased by about 50 to about 150 ml after administration. In some embodiments, the subject's FVC is increased by about 100 ml after administration. In some embodiments, the subject's FVC is increased by about 200 mL to about 500 mL after administration. In some embodiments, the subject's FVC is increased by about 250 mL to about 500 mL after administration. In some embodiments, the subject's FVC is increased by about 300 mL to about 500 mL after administration. In some embodiments, the subject's FVC is increased by about 350 mL to about 500 mL after administration. In some embodiments, the subject's FVC is increased by about 400 mL to about 500 mL after administration. In some embodiments, the subject's FVC is increased by about 450 mL to about 500 mL after administration. In some embodiments, the subject's FVC is increased by about 500 mL to about 900 mL. In some embodiments, the subject's FVC is increased by about 550 mL to about 900 mL. In some embodiments, the subject's FVC is increased by about 600 mL to about 900 mL. In some embodiments, the subject's FVC is increased by about 650 mL to about 900 mL. In some embodiments, the subject's FVC is increased by about 700 mL to about 900 mL. In some embodiments, the subject's FVC is increased by about 750 mL to about 900 mL. In some embodiments, the subject's FVC is increased by about 800 mL to about 900 mL. In some embodiments, the subject's FVC is increased by about 850 mL to about 900 mL. In some embodiments FVC is measured pre-bronchodilation. In some embodiments FVC is measured post-bronchodilation.

In some embodiments, administering dexpramipexole, or a pharmaceutically acceptable salt thereof, improves the subject's FEV₁/FVC. In some embodiments, the subject's FEV₁/FVC is increased by about 0.1 or greater. In some embodiments, the subject's FEV₁/FVC is increased by about 0.2 or greater. In some embodiments FEV₁/FVC is measured pre-bronchodilation. In some embodiments FEV₁/FVC is measured post-bronchodilation.

In some embodiments, administering dexpramipexole, or a pharmaceutically acceptable salt thereof, improves the subject's forced mid-expiratory flow (FEF₂₅₋₇₅). In some embodiments FEF₂₅₋₇₅ is measured pre-bronchodilation. In some embodiments FEF₂₅₋₇₅ is measured post-bronchodilation.

In some embodiments, administering dexpramipexole, or a pharmaceutically acceptable salt thereof, improves the subject's ratio of forced expiratory volume in 3 seconds to FVC (FEV₃/FVC). In some embodiments FEV₃/FVC is measured pre-bronchodilation. In some embodiments FEV₃/FVC is measured post-bronchodilation.

In some embodiments, administering dexpramipexole, or a pharmaceutically acceptable salt thereof, improves a measurement selected from the group consisting of St George's Respiratory Questionnaire for COPD Patients (SGRQ-C), COPD Assessment Test (CAT), Modified Medical Research Council Grading System (mMRC), Evaluating Respiratory Symptoms in Chronic Obstructive Pulmonary Disease (E-RS:COPD), and any combination thereof.

In some embodiments, administering dexpramipexole, or a pharmaceutically acceptable salt thereof, improves the subject's SGRQ-C total score measurement. In some embodiments the SGRQ-C measurement improves by about 1 to about 40 points. In some embodiments the SGRQ-C measurement improves by about 10 to about 15 points. In some embodiments the SGRQ-C measurement improves by about 10 to about 20 points. In some embodiments the SGRQ-C measurement improves by about 20 to about 30 points. In some embodiments the SGRQ-C measurement improves by about 20 to about 40 points. In some embodiments the SGRQ-C measurement improves by about 5 to about 10 points. In some embodiments the SGRQ-C measurement improves by about 10 to about 15 points. In some embodiments the SGRQ-C measurement improves by about 15 to about 20 points.

In some embodiments, administering dexpramipexole, or a pharmaceutically acceptable salt thereof, improves the subject's CAT total score measurement. In some embodiments the CAT measurement improves by about 1 to about 30 points. In some embodiments the CAT measurement improves by about 10 to about 15 points. In some embodiments the CAT measurement improves by about 10 to about 20 points. In some embodiments the CAT measurement improves by about 20 to about 30 points. In some embodiments the CAT measurement improves by about 5 to about 10 points. In some embodiments the CAT measurement improves by about 10 to about 15 points. In some embodiments the CAT measurement improves by about 15 to about 20 points.

In some embodiments, prior to treatment with dexpramipexole or a pharmaceutically acceptable salt thereof, the subject is already receiving a treatment regimen comprising administration of an inhaled corticosteroid (ICS), thereby treating the COPD in the subject. In some embodiments, the treatment regimen comprises administration of an ICS and a long-acting 32 agonist (LABA). In some embodiments, the treatment regimen comprises administration of an ICS and a long-acting muscarinic antagonist (LAMA). In some embodiments, the treatment regimen comprises administration of an ICS, LABA, and LAMA. In some embodiments, the subject has received the treatment regimen for at least 12 weeks. In some embodiments, the subject has received a stable dose of the treatment regimen for at least 4 weeks. In some embodiments, the ICS is selected from the group consisting of beclomethasone, fluticasone, ciclesonide, mometasone, budesonide, flunisolide, and any combination thereof. In some embodiments, the LABA is selected from the group consisting of formoterol fumarate, salmeterol, salmeterol xinafoate, arformoterol tartrate, olodaterol, vilanterol, indacaterol, and any combination thereof. In some embodiments, the LAMA is selected from the group consisting of aclidinium, glycopyrrolate, glycopyrronium, tiotropium, umeclidinium, and any combination thereof. In some embodiments, prior to treatment with dexpramipexole or a pharmaceutically acceptable salt thereof, the subject is receiving one or more COPD medications selected from the group consisting of short-acting bronchodilators, long-acting bronchodilators, ultra-long-acting bronchodilators, xanthines, short-acting muscarinic antagonists, long-acting muscarinic antagonists, leukotriene antagonists, 5-lipoxygenase inhibitors, and any combination thereof.

In some embodiments, the subject is a current or former smoker. In some embodiments, the subject's COPD is from toxic exposure.

Further provided herein are methods of reducing rate of hospitalization due to exacerbations in a human subject with eosinophilic chronic obstructive pulmonary disorder (COPD), comprising orally administering to the subject a therapeutically effective amount of dexpramipexole, or a pharmaceutically acceptable salt thereof.

In some embodiments, dexpramipexole, or a pharmaceutically acceptable salt thereof, is administered in a daily dose of about 150 mg to about 300 mg per day. In some embodiments, dexpramipexole, or a pharmaceutically acceptable salt thereof, is administered in a daily dose of about 300 mg per day. In some embodiments, dexpramipexole, or a pharmaceutically acceptable salt thereof, is administered as about 300 mg once per day. In some embodiments, dexpramipexole, or a pharmaceutically acceptable salt thereof, is administered as about 150 mg twice daily. In some embodiments, dexpramipexole, or a pharmaceutically acceptable salt thereof, is administered in a daily dose of about 150 mg per day. In some embodiments, dexpramipexole, or a pharmaceutically acceptable salt thereof, is administered as about 150 mg once per day. In some embodiments, dexpramipexole, or a pharmaceutically acceptable salt thereof, is administered as about 75 mg twice daily.

In some embodiments, the subject has a blood absolute eosinophil count (AEC) of ≥0.15×10⁹/L before administration of dexpramipexole, or a pharmaceutically acceptable salt thereof. In some embodiments, the subject has a blood absolute eosinophil count (AEC) of ≥0.30×10⁹/L before administration of dexpramipexole, or a pharmaceutically acceptable salt thereof.

In some embodiments, the subject has evidence of mucus plugs (i.e., a mucus plug score of ≥1) before administration of dexpramipexole, or a pharmaceutically acceptable salt thereof. In some embodiments, administering dexpramipexole, or a pharmaceutically acceptable salt thereof, reduces the presence of mucus plugs as demonstrated by reducing the mucus plug score.

In some embodiments, administering dexpramipexole, or a pharmaceutically acceptable salt thereof, improves the subject's mean forced expiratory volume in 1 second (FEV₁). In some embodiments, the subject's FEV₁ is increased by about 5% to about 50%. In some embodiments, the subject's FEV₁ is increased by about 5% to about 10%. In some embodiments, the subject's FEV₁ is increased by about 10% to about 30%. In some embodiments, the subject's FEV₁ is increased by about 30% to about 50%. In some embodiments, the subject's FEV₁ is increased by about 16% to about 30%. In some embodiments, the subject's FEV₁ is increased by about 18% to about 30%. In some embodiments, the subject's FEV₁ is increased by about 20% to about 30%. In some embodiments, the subject's FEV₁ is increased by about 20% to about 30%. In some embodiments, the subject's FEV₁ is increased by about 22% to about 30%. In some embodiments, the subject's FEV₁ is increased by about 24% to about 30%. In some embodiments, the subject's FEV₁ is increased by about 26% to about 30%. In some embodiments, the subject's FEV₁ is increased by about 28% to about 30%. In some embodiments, the subject's FEV₁ is increased by about 30% to about 75%. In some embodiments, the subject's FEV₁ is increased by about 35% to about 75%. In some embodiments, the subject's FEV₁ is increased by about 40% to about 75%. In some embodiments, the subject's FEV₁ is increased by about 45% to about 75%. In some embodiments, the subject's FEV₁ is increased by about 55% to about 75%. In some embodiments, the subject's FEV₁ is increased by about 65% to about 75%. In some embodiments FEV₁ is measured pre-bronchodilation. In some embodiments FEV₁ is measured post-bronchodilation.

In some embodiments, the subject's FEV₁ is increased by about 30 to about 1000 ml. In some embodiments, the subject's FEV₁ is increased by about 40 to about 500 ml after administration. In some embodiments, the subject's FEV₁ is increased by about 50 to about 150 ml after administration. In some embodiments, the subject's FEV₁ is increased by about 100 ml after administration. In some embodiments, the subject's FEV₁ is increased by about 200 mL to about 500 mL after administration. In some embodiments, the subject's FEV₁ is increased by about 250 mL to about 500 mL after administration. In some embodiments, the subject's FEV₁ is increased by about 300 mL to about 500 mL after administration. In some embodiments, the subject's FEV₁ is increased by about 350 mL to about 500 mL after administration. In some embodiments, the subject's FEV₁ is increased by about 400 mL to about 500 mL after administration. In some embodiments, the subject's FEV₁ is increased by about 450 mL to about 500 mL after administration. In some embodiments, the subject's FEV₁ is increased by about 500 mL to about 900 mL. In some embodiments, the subject's FEV₁ is increased by about 550 mL to about 900 mL. In some embodiments, the subject's FEV₁ is increased by about 600 mL to about 900 mL. In some embodiments, the subject's FEV₁ is increased by about 650 mL to about 900 mL. In some embodiments, the subject's FEV₁ is increased by about 700 mL to about 900 mL. In some embodiments, the subject's FEV₁ is increased by about 750 mL to about 900 mL. In some embodiments, the subject's FEV₁ is increased by about 800 mL to about 900 mL. In some embodiments, the subject's FEV₁ is increased by about 850 mL to about 900 mL. In some embodiments FEV₁ is measured pre-bronchodilation. In some embodiments FEV₁ is measured post-bronchodilation.

In some embodiments, administering dexpramipexole, or a pharmaceutically acceptable salt thereof, improves the subject's forced vital capacity (FVC). In some embodiments, the subject's FVC is increased by about 5% to about 50%. In some embodiments, the subject's FVC is increased by about 5% to about 10%. In some embodiments, the subject's FVC is increased by about 10% to about 30%. In some embodiments, the subject's FVC is increased by about 30% to about 50%. In some embodiments, the subject's FVC is increased by about 6% to about 20%. In some embodiments, the subject's FVC is increased by about 8% to about 20%. In some embodiments, the subject's FVC is increased by about 10% to about 20%. In some embodiments, the subject's FVC is increased by about 12% to about 20%. In some embodiments, the subject's FVC is increased by about 14% to about 20%. In some embodiments, the subject's FVC is increased by about 16% to about 20%. In some embodiments, the subject's FVC is increased by about 18% to about 20%. In some embodiments, the subject's FVC is increased by about 10% to about 40%. In some embodiments, the subject's FVC is increased by about 15% to about 40%. In some embodiments, the subject's FVC is increased by about 20% to about 40%. In some embodiments, the subject's FVC is increased by about 25% to about 40%. In some embodiments, the subject's FVC is increased by about 30% to about 40%. In some embodiments, the subject's FVC is increased by about 35% to about 40%. In some embodiments FVC is measured pre-bronchodilation. In some embodiments FVC is measured post-bronchodilation.

In some embodiments, the subject's FVC is increased by about 30 ml to about 1000 ml after administration. In some embodiments, the subject's FVC is increased by about 40 to about 500 ml after administration. In some embodiments, the subject's FVC is increased by about 50 to about 150 ml after administration. In some embodiments, the subject's FVC is increased by about 100 ml after administration. In some embodiments, the subject's FVC is increased by about 200 mL to about 500 mL after administration. In some embodiments, the subject's FVC is increased by about 250 mL to about 500 mL after administration. In some embodiments, the subject's FVC is increased by about 300 mL to about 500 mL after administration. In some embodiments, the subject's FVC is increased by about 350 mL to about 500 mL after administration. In some embodiments, the subject's FVC is increased by about 400 mL to about 500 mL after administration. In some embodiments, the subject's FVC is increased by about 450 mL to about 500 mL after administration. In some embodiments, the subject's FVC is increased by about 500 mL to about 900 mL. In some embodiments, the subject's FVC is increased by about 550 mL to about 900 mL. In some embodiments, the subject's FVC is increased by about 600 mL to about 900 mL. In some embodiments, the subject's FVC is increased by about 650 mL to about 900 mL. In some embodiments, the subject's FVC is increased by about 700 mL to about 900 mL. In some embodiments, the subject's FVC is increased by about 750 mL to about 900 mL. In some embodiments, the subject's FVC is increased by about 800 mL to about 900 mL. In some embodiments, the subject's FVC is increased by about 850 mL to about 900 mL. In some embodiments FVC is measured pre-bronchodilation. In some embodiments FVC is measured post-bronchodilation.

In some embodiments, administering dexpramipexole, or a pharmaceutically acceptable salt thereof, improves the subject's FEV₁/FVC. In some embodiments, the subject's FEV₁/FVC is increased by about 0.1 or greater. In some embodiments, the subject's FEV₁/FVC is increased by about 0.2 or greater. In some embodiments FEV₁/FVC is measured pre-bronchodilation. In some embodiments FEV₁/FVC is measured post-bronchodilation.

In some embodiments, administering dexpramipexole, or a pharmaceutically acceptable salt thereof, improves the subject's forced mid-expiratory flow (FEF₂₅₋₇₅). In some embodiments FEF₂₅₋₇₅ is measured pre-bronchodilation. In some embodiments FEF₂₅₋₇₅ is measured post-bronchodilation.

In some embodiments, administering dexpramipexole, or a pharmaceutically acceptable salt thereof, improves the subject's ratio of forced expiratory volume in 3 seconds to FVC (FEV₃/FVC). In some embodiments FEV₃/FVC is measured pre-bronchodilation. In some embodiments FEV₃/FVC is measured post-bronchodilation.

In some embodiments, administering dexpramipexole, or a pharmaceutically acceptable salt thereof, improves a measurement selected from the group consisting of St George's Respiratory Questionnaire for COPD Patients (SGRQ-C), COPD Assessment Test (CAT), Modified Medical Research Council Grading System (mMRC), Evaluating Respiratory Symptoms in Chronic Obstructive Pulmonary Disease (E-RS:COPD), and any combination thereof.

In some embodiments, administering dexpramipexole, or a pharmaceutically acceptable salt thereof, improves the subject's SGRQ-C total score measurement. In some embodiments the SGRQ-C measurement improves by about 1 to about 40 points. In some embodiments the SGRQ-C measurement improves by about 10 to about 15 points. In some embodiments the SGRQ-C measurement improves by about 10 to about 20 points. In some embodiments the SGRQ-C measurement improves by about 20 to about 30 points. In some embodiments the SGRQ-C measurement improves by about 20 to about 40 points. In some embodiments the SGRQ-C measurement improves by about 5 to about 10 points. In some embodiments the SGRQ-C measurement improves by about 10 to about 15 points. In some embodiments the SGRQ-C measurement improves by about 15 to about 20 points.

In some embodiments, administering dexpramipexole, or a pharmaceutically acceptable salt thereof, improves the subject's CAT total score measurement. In some embodiments the CAT measurement improves by about 1 to about 30 points. In some embodiments the CAT measurement improves by about 10 to about 15 points. In some embodiments the CAT measurement improves by about 10 to about 20 points. In some embodiments the CAT measurement improves by about 20 to about 30 points. In some embodiments the CAT measurement improves by about 5 to about 10 points. In some embodiments the CAT measurement improves by about 10 to about 15 points. In some embodiments the CAT measurement improves by about 15 to about 20 points.

In some embodiments, prior to treatment with dexpramipexole or a pharmaceutically acceptable salt thereof, the subject is already receiving a treatment regimen comprising administration of an inhaled corticosteroid (ICS), thereby treating the COPD in the subject. In some embodiments, the treatment regimen comprises administration of an ICS and a long-acting 32 agonist (LABA). In some embodiments, the treatment regimen comprises administration of an ICS and a long-acting muscarinic antagonist (LAMA). In some embodiments, the treatment regimen comprises administration of an ICS, LABA, and LAMA. In some embodiments, the subject has received the treatment regimen for at least 12 weeks. In some embodiments, the subject has received a stable dose of the treatment regimen for at least 4 weeks. In some embodiments, the ICS is selected from the group consisting of beclomethasone, fluticasone, ciclesonide, mometasone, budesonide, flunisolide, and any combination thereof. In some embodiments, the LABA is selected from the group consisting of formoterol fumarate, salmeterol, salmeterol xinafoate, arformoterol tartrate, olodaterol, vilanterol, indacaterol, and any combination thereof. In some embodiments, the LAMA is selected from the group consisting of aclidinium, glycopyrrolate, glycopyrronium, tiotropium, umeclidinium, and any combination thereof. In some embodiments, prior to treatment with dexpramipexole or a pharmaceutically acceptable salt thereof, the subject is receiving one or more COPD medications selected from the group consisting of short-acting bronchodilators, long-acting bronchodilators, ultra-long-acting bronchodilators, xanthines, short-acting muscarinic antagonists, long-acting muscarinic antagonists, leukotriene antagonists, 5-lipoxygenase inhibitors, and any combination thereof.

In some embodiments, the subject is a current or former smoker. In some embodiments, the subject's COPD is from toxic exposure.

Further provided herein are methods of reducing the rate of oral/parenteral steroid and/or antibiotic use for treatment of COPD in a human subject with eosinophilic chronic obstructive pulmonary disorder (COPD), comprising orally administering to the subject a therapeutically effective amount of dexpramipexole, or a pharmaceutically acceptable salt thereof.

In some embodiments, dexpramipexole, or a pharmaceutically acceptable salt thereof, is administered in a daily dose of about 150 mg to about 300 mg per day. In some embodiments, dexpramipexole, or a pharmaceutically acceptable salt thereof, is administered in a daily dose of about 300 mg per day. In some embodiments, dexpramipexole, or a pharmaceutically acceptable salt thereof, is administered as about 300 mg once per day. In some embodiments, dexpramipexole, or a pharmaceutically acceptable salt thereof, is administered as about 150 mg twice daily. In some embodiments, dexpramipexole, or a pharmaceutically acceptable salt thereof, is administered in a daily dose of about 150 mg per day. In some embodiments, dexpramipexole, or a pharmaceutically acceptable salt thereof, is administered as about 150 mg once per day. In some embodiments, dexpramipexole, or a pharmaceutically acceptable salt thereof, is administered as about 75 mg twice daily.

In some embodiments, the subject has a blood absolute eosinophil count (AEC) of ≥0.15×10⁹/L before administration of dexpramipexole, or a pharmaceutically acceptable salt thereof. In some embodiments, the subject has a blood absolute eosinophil count (AEC) of ≥0.30×10⁹/L before administration of dexpramipexole, or a pharmaceutically acceptable salt thereof.

In some embodiments, the subject has evidence of mucus plugs (i.e., a mucus plug score of ≥1) before administration of dexpramipexole, or a pharmaceutically acceptable salt thereof. In some embodiments, administering dexpramipexole, or a pharmaceutically acceptable salt thereof, reduces the presence of mucus plugs as demonstrated by reducing the mucus plug score.

In some embodiments, administering dexpramipexole, or a pharmaceutically acceptable salt thereof, improves the subject's mean forced expiratory volume in 1 second (FEV₁). In some embodiments, the subject's FEV₁ is increased by about 5% to about 50%. In some embodiments, the subject's FEV₁ is increased by about 5% to about 10%. In some embodiments, the subject's FEV₁ is increased by about 10% to about 30%. In some embodiments, the subject's FEV₁ is increased by about 30% to about 50%. In some embodiments, the subject's FEV₁ is increased by about 16% to about 30%. In some embodiments, the subject's FEV₁ is increased by about 18% to about 30%. In some embodiments, the subject's FEV₁ is increased by about 20% to about 30%. In some embodiments, the subject's FEV₁ is increased by about 22% to about 30%. In some embodiments, the subject's FEV₁ is increased by about 24% to about 30%. In some embodiments, the subject's FEV₁ is increased by about 26% to about 30%. In some embodiments, the subject's FEV₁ is increased by about 28% to about 30%. In some embodiments, the subject's FEV₁ is increased by about 30% to about 75%. In some embodiments, the subject's FEV₁ is increased by about 35% to about 75%. In some embodiments, the subject's FEV₁ is increased by about 40% to about 75%. In some embodiments, the subject's FEV₁ is increased by about 45% to about 75%. In some embodiments, the subject's FEV₁ is increased by about 55% to about 75%. In some embodiments, the subject's FEV₁ is increased by about 65% to about 75%. In some embodiments FEV₁ is measured pre-bronchodilation. In some embodiments FEV₁ is measured post-bronchodilation.

In some embodiments, the subject's FEV₁ is increased by about 30 to about 1000 ml. In some embodiments, the subject's FEV₁ is increased by about 40 to about 500 ml after administration. In some embodiments, the subject's FEV₁ is increased by about 50 to about 150 ml after administration. In some embodiments, the subject's FEV₁ is increased by about 100 ml after administration. In some embodiments, the subject's FEV₁ is increased by about 200 mL to about 500 mL after administration. In some embodiments, the subject's FEV₁ is increased by about 250 mL to about 500 mL after administration. In some embodiments, the subject's FEV₁ is increased by about 300 mL to about 500 mL after administration. In some embodiments, the subject's FEV₁ is increased by about 350 mL to about 500 mL after administration. In some embodiments, the subject's FEV₁ is increased by about 400 mL to about 500 mL after administration. In some embodiments, the subject's FEV₁ is increased by about 450 mL to about 500 mL after administration. In some embodiments, the subject's FEV₁ is increased by about 500 mL to about 900 mL. In some embodiments, the subject's FEV₁ is increased by about 550 mL to about 900 mL. In some embodiments, the subject's FEV₁ is increased by about 600 mL to about 900 mL. In some embodiments, the subject's FEV₁ is increased by about 650 mL to about 900 mL. In some embodiments, the subject's FEV₁ is increased by about 700 mL to about 900 mL. In some embodiments, the subject's FEV₁ is increased by about 750 mL to about 900 mL. In some embodiments, the subject's FEV₁ is increased by about 800 mL to about 900 mL. In some embodiments, the subject's FEV₁ is increased by about 850 mL to about 900 mL. In some embodiments FEV₁ is measured pre-bronchodilation. In some embodiments FEV₁ is measured post-bronchodilation.

In some embodiments, administering dexpramipexole, or a pharmaceutically acceptable salt thereof, improves the subject's forced vital capacity (FVC). In some embodiments, the subject's FVC is increased by about 5% to about 50%. In some embodiments, the subject's FVC is increased by about 5% to about 10%. In some embodiments, the subject's FVC is increased by about 10% to about 30%. In some embodiments, the subject's FVC is increased by about 30% to about 50%. In some embodiments, the subject's FVC is increased by about 6% to about 20%. In some embodiments, the subject's FVC is increased by about 8% to about 20%. In some embodiments, the subject's FVC is increased by about 10% to about 20%. In some embodiments, the subject's FVC is increased by about 12% to about 20%. In some embodiments, the subject's FVC is increased by about 14% to about 20%. In some embodiments, the subject's FVC is increased by about 16% to about 20%. In some embodiments, the subject's FVC is increased by about 18% to about 20%. In some embodiments, the subject's FVC is increased by about 10% to about 40%. In some embodiments, the subject's FVC is increased by about 15% to about 40%. In some embodiments, the subject's FVC is increased by about 20% to about 40%. In some embodiments, the subject's FVC is increased by about 25% to about 40%. In some embodiments, the subject's FVC is increased by about 30% to about 40%. In some embodiments, the subject's FVC is increased by about 35% to about 40%. In some embodiments FVC is measured pre-bronchodilation. In some embodiments FVC is measured post-bronchodilation.

In some embodiments, the subject's FVC is increased by about 30 ml to about 1000 ml after administration. In some embodiments, the subject's FVC is increased by about 40 to about 500 ml after administration. In some embodiments, the subject's FVC is increased by about 50 to about 150 ml after administration. In some embodiments, the subject's FVC is increased by about 100 ml after administration. In some embodiments, the subject's FVC is increased by about 200 mL to about 500 mL after administration. In some embodiments, the subject's FVC is increased by about 250 mL to about 500 mL after administration. In some embodiments, the subject's FVC is increased by about 300 mL to about 500 mL after administration. In some embodiments, the subject's FVC is increased by about 350 mL to about 500 mL after administration. In some embodiments, the subject's FVC is increased by about 400 mL to about 500 mL after administration. In some embodiments, the subject's FVC is increased by about 450 mL to about 500 mL after administration. In some embodiments, the subject's FVC is increased by about 500 mL to about 900 mL. In some embodiments, the subject's FVC is increased by about 550 mL to about 900 mL. In some embodiments, the subject's FVC is increased by about 600 mL to about 900 mL. In some embodiments, the subject's FVC is increased by about 650 mL to about 900 mL. In some embodiments, the subject's FVC is increased by about 700 mL to about 900 mL. In some embodiments, the subject's FVC is increased by about 750 mL to about 900 mL. In some embodiments, the subject's FVC is increased by about 800 mL to about 900 mL. In some embodiments, the subject's FVC is increased by about 850 mL to about 900 mL. In some embodiments FVC is measured pre-bronchodilation. In some embodiments FVC is measured post-bronchodilation.

In some embodiments, administering dexpramipexole, or a pharmaceutically acceptable salt thereof, improves the subject's FEV₁/FVC. In some embodiments, the subject's FEV₁/FVC is increased by about 0.1 or greater. In some embodiments, the subject's FEV₁/FVC is increased by about 0.2 or greater. In some embodiments FEV₁/FVC is measured pre-bronchodilation. In some embodiments FEV₁/FVC is measured post-bronchodilation.

In some embodiments, administering dexpramipexole, or a pharmaceutically acceptable salt thereof, improves the subject's forced mid-expiratory flow (FEF₂₅₋₇₅). In some embodiments FEF₂₅₋₇₅ is measured pre-bronchodilation. In some embodiments FEF₂₅₋₇₅ is measured post-bronchodilation.

In some embodiments, administering dexpramipexole, or a pharmaceutically acceptable salt thereof, improves the subject's ratio of forced expiratory volume in 3 seconds to FVC (FEV₃/FVC). In some embodiments FEV₃/FVC is measured pre-bronchodilation. In some embodiments FEV₃/FVC is measured post-bronchodilation.

In some embodiments, administering dexpramipexole, or a pharmaceutically acceptable salt thereof, improves a measurement selected from the group consisting of St George's Respiratory Questionnaire for COPD Patients (SGRQ-C), COPD Assessment Test (CAT), Modified Medical Research Council Grading System (mMRC), Evaluating Respiratory Symptoms in Chronic Obstructive Pulmonary Disease (E-RS:COPD), and any combination thereof.

In some embodiments, administering dexpramipexole, or a pharmaceutically acceptable salt thereof, improves the subject's SGRQ-C total score measurement. In some embodiments the SGRQ-C measurement improves by about 1 to about 40 points. In some embodiments the SGRQ-C measurement improves by about 10 to about 15 points. In some embodiments the SGRQ-C measurement improves by about 10 to about 20 points. In some embodiments the SGRQ-C measurement improves by about 20 to about 30 points. In some embodiments the SGRQ-C measurement improves by about 20 to about 40 points. In some embodiments the SGRQ-C measurement improves by about 5 to about 10 points. In some embodiments the SGRQ-C measurement improves by about 10 to about 15 points. In some embodiments the SGRQ-C measurement improves by about 15 to about 20 points.

In some embodiments, administering dexpramipexole, or a pharmaceutically acceptable salt thereof, improves the subject's CAT total score measurement. In some embodiments the CAT measurement improves by about 1 to about 30 points. In some embodiments the CAT measurement improves by about 10 to about 15 points. In some embodiments the CAT measurement improves by about 10 to about 20 points. In some embodiments the CAT measurement improves by about 20 to about 30 points. In some embodiments the CAT measurement improves by about 5 to about 10 points. In some embodiments the CAT measurement improves by about 10 to about 15 points. In some embodiments the CAT measurement improves by about 15 to about 20 points.

In some embodiments, prior to treatment with dexpramipexole or a pharmaceutically acceptable salt thereof, the subject is already receiving a treatment regimen comprising administration of an inhaled corticosteroid (ICS), thereby treating the COPD in the subject. In some embodiments, the treatment regimen comprises administration of an ICS and a long-acting 32 agonist (LABA). In some embodiments, the treatment regimen comprises administration of an ICS and a long-acting muscarinic antagonist (LAMA). In some embodiments, the treatment regimen comprises administration of an ICS, LABA, and LAMA. In some embodiments, the subject has received the treatment regimen for at least 12 weeks. In some embodiments, the subject has received a stable dose of the treatment regimen for at least 4 weeks. In some embodiments, the ICS is selected from the group consisting of beclomethasone, fluticasone, ciclesonide, mometasone, budesonide, flunisolide, and any combination thereof. In some embodiments, the LABA is selected from the group consisting of formoterol fumarate, salmeterol, salmeterol xinafoate, arformoterol tartrate, olodaterol, vilanterol, indacaterol, and any combination thereof. In some embodiments, the LAMA is selected from the group consisting of aclidinium, glycopyrrolate, glycopyrronium, tiotropium, umeclidinium, and any combination thereof. In some embodiments, prior to treatment with dexpramipexole or a pharmaceutically acceptable salt thereof, the subject is receiving one or more COPD medications selected from the group consisting of short-acting bronchodilators, long-acting bronchodilators, ultra-long-acting bronchodilators, xanthines, short-acting muscarinic antagonists, long-acting muscarinic antagonists, leukotriene antagonists, 5-lipoxygenase inhibitors, and any combination thereof.

In some embodiments, the subject is a current or former smoker. In some embodiments, the subject's COPD is from toxic exposure.

Further provided herein are methods of changing the level of one or more biomarkers associated with eosinophilic chronic obstructive pulmonary disorder (COPD) or lung function decline in a human subject with eosinophilic COPD, comprising orally administering to the subject a therapeutically effective amount of dexpramipexole, or a pharmaceutically acceptable salt thereof.

In some embodiments, dexpramipexole, or a pharmaceutically acceptable salt thereof, is administered in a daily dose of about 150 mg to about 300 mg per day. In some embodiments, dexpramipexole, or a pharmaceutically acceptable salt thereof, is administered in a daily dose of about 300 mg per day. In some embodiments, dexpramipexole, or a pharmaceutically acceptable salt thereof, is administered as about 300 mg once per day. In some embodiments, dexpramipexole, or a pharmaceutically acceptable salt thereof, is administered as about 150 mg twice daily. In some embodiments, dexpramipexole, or a pharmaceutically acceptable salt thereof, is administered in a daily dose of about 150 mg per day. In some embodiments, dexpramipexole, or a pharmaceutically acceptable salt thereof, is administered as about 150 mg once per day. In some embodiments, dexpramipexole, or a pharmaceutically acceptable salt thereof, is administered as about 75 mg twice daily.

In some embodiments, the subject has a blood absolute eosinophil count (AEC) of ≥0.15×10⁹/L before administration of dexpramipexole, or a pharmaceutically acceptable salt thereof. In some embodiments, the subject has a blood absolute eosinophil count (AEC) of ≥0.30×10⁹/L before administration of dexpramipexole, or a pharmaceutically acceptable salt thereof.

In some embodiments, the subject has evidence of mucus plugs (i.e., a mucus plug score of ≥1) before administration of dexpramipexole, or a pharmaceutically acceptable salt thereof. In some embodiments, administering dexpramipexole, or a pharmaceutically acceptable salt thereof, reduces the presence of mucus plugs as demonstrated by reducing the mucus plug score.

In some embodiments, administering dexpramipexole, or a pharmaceutically acceptable salt thereof, improves the subject's mean forced expiratory volume in 1 second (FEV₁). In some embodiments, the subject's FEV₁ is increased by about 5% to about 50%. In some embodiments, the subject's FEV₁ is increased by about 5% to about 10%. In some embodiments, the subject's FEV₁ is increased by about 10% to about 30%. In some embodiments, the subject's FEV₁ is increased by about 30% to about 50%. In some embodiments, the subject's FEV₁ is increased by about 16% to about 30%. In some embodiments, the subject's FEV₁ is increased by about 18% to about 30%. In some embodiments, the subject's FEV₁ is increased by about 20% to about 30%. In some embodiments, the subject's FEV₁ is increased by about 22% to about 30%. In some embodiments, the subject's FEV₁ is increased by about 24% to about 30%. In some embodiments, the subject's FEV₁ is increased by about 26% to about 30%. In some embodiments, the subject's FEV₁ is increased by about 28% to about 30%. In some embodiments, the subject's FEV₁ is increased by about 30% to about 75%. In some embodiments, the subject's FEV₁ is increased by about 35% to about 75%. In some embodiments, the subject's FEV₁ is increased by about 40% to about 75%. In some embodiments, the subject's FEV₁ is increased by about 45% to about 75%. In some embodiments, the subject's FEV₁ is increased by about 55% to about 75%. In some embodiments, the subject's FEV₁ is increased by about 65% to about 75%. In some embodiments FEV₁ is measured pre-bronchodilation. In some embodiments FEV₁ is measured post-bronchodilation.

In some embodiments, the subject's FEV₁ is increased by about 30 to about 1000 ml. In some embodiments, the subject's FEV₁ is increased by about 40 to about 500 ml after administration. In some embodiments, the subject's FEV₁ is increased by about 50 to about 150 ml after administration. In some embodiments, the subject's FEV₁ is increased by about 100 ml after administration. In some embodiments, the subject's FEV₁ is increased by about 200 mL to about 500 mL after administration. In some embodiments, the subject's FEV₁ is increased by about 250 mL to about 500 mL after administration. In some embodiments, the subject's FEV₁ is increased by about 300 mL to about 500 mL after administration. In some embodiments, the subject's FEV₁ is increased by about 350 mL to about 500 mL after administration. In some embodiments, the subject's FEV₁ is increased by about 400 mL to about 500 mL after administration. In some embodiments, the subject's FEV₁ is increased by about 450 mL to about 500 mL after administration. In some embodiments, the subject's FEV₁ is increased by about 500 mL to about 900 mL. In some embodiments, the subject's FEV₁ is increased by about 550 mL to about 900 mL. In some embodiments, the subject's FEV₁ is increased by about 600 mL to about 900 mL. In some embodiments, the subject's FEV₁ is increased by about 650 mL to about 900 mL. In some embodiments, the subject's FEV₁ is increased by about 700 mL to about 900 mL. In some embodiments, the subject's FEV₁ is increased by about 750 mL to about 900 mL. In some embodiments, the subject's FEV₁ is increased by about 800 mL to about 900 mL. In some embodiments, the subject's FEV₁ is increased by about 850 mL to about 900 mL. In some embodiments FEV₁ is measured pre-bronchodilation. In some embodiments FEV₁ is measured post-bronchodilation.

In some embodiments, administering dexpramipexole, or a pharmaceutically acceptable salt thereof, improves the subject's forced vital capacity (FVC). In some embodiments, the subject's FVC is increased by about 5% to about 50%. In some embodiments, the subject's FVC is increased by about 5% to about 10%. In some embodiments, the subject's FVC is increased by about 10% to about 30%. In some embodiments, the subject's FVC is increased by about 30% to about 50%. In some embodiments, the subject's FVC is increased by about 6% to about 20%. In some embodiments, the subject's FVC is increased by about 8% to about 20%. In some embodiments, the subject's FVC is increased by about 10% to about 20%. In some embodiments, the subject's FVC is increased by about 12% to about 20%. In some embodiments, the subject's FVC is increased by about 14% to about 20%. In some embodiments, the subject's FVC is increased by about 16% to about 20%. In some embodiments, the subject's FVC is increased by about 18% to about 20%. In some embodiments, the subject's FVC is increased by about 10% to about 40%. In some embodiments, the subject's FVC is increased by about 15% to about 40%. In some embodiments, the subject's FVC is increased by about 20% to about 40%. In some embodiments, the subject's FVC is increased by about 25% to about 40%. In some embodiments, the subject's FVC is increased by about 30% to about 40%. In some embodiments, the subject's FVC is increased by about 35% to about 40%. In some embodiments FVC is measured pre-bronchodilation. In some embodiments FVC is measured post-bronchodilation.

In some embodiments, the subject's FVC is increased by about 30 ml to about 1000 ml after administration. In some embodiments, the subject's FVC is increased by about 40 to about 500 ml after administration. In some embodiments, the subject's FVC is increased by about 50 to about 150 ml after administration. In some embodiments, the subject's FVC is increased by about 100 ml after administration. In some embodiments, the subject's FVC is increased by about 200 mL to about 500 mL after administration. In some embodiments, the subject's FVC is increased by about 250 mL to about 500 mL after administration. In some embodiments, the subject's FVC is increased by about 300 mL to about 500 mL after administration. In some embodiments, the subject's FVC is increased by about 350 mL to about 500 mL after administration. In some embodiments, the subject's FVC is increased by about 400 mL to about 500 mL after administration. In some embodiments, the subject's FVC is increased by about 450 mL to about 500 mL after administration. In some embodiments, the subject's FVC is increased by about 500 mL to about 900 mL. In some embodiments, the subject's FVC is increased by about 550 mL to about 900 mL. In some embodiments, the subject's FVC is increased by about 600 mL to about 900 mL. In some embodiments, the subject's FVC is increased by about 650 mL to about 900 mL. In some embodiments, the subject's FVC is increased by about 700 mL to about 900 mL. In some embodiments, the subject's FVC is increased by about 750 mL to about 900 mL. In some embodiments, the subject's FVC is increased by about 800 mL to about 900 mL. In some embodiments, the subject's FVC is increased by about 850 mL to about 900 mL. In some embodiments FVC is measured pre-bronchodilation. In some embodiments FVC is measured post-bronchodilation.

In some embodiments, administering dexpramipexole, or a pharmaceutically acceptable salt thereof, improves the subject's FEV₁/FVC. In some embodiments, the subject's FEV₁/FVC is increased by about 0.1 or greater. In some embodiments, the subject's FEV₁/FVC is increased by about 0.2 or greater. In some embodiments FEV₁/FVC is measured pre-bronchodilation. In some embodiments FEV₁/FVC is measured post-bronchodilation.

In some embodiments, administering dexpramipexole, or a pharmaceutically acceptable salt thereof, improves the subject's forced mid-expiratory flow (FEF₂₅₋₇₅). In some embodiments FEF₂₅₋₇₅ is measured pre-bronchodilation. In some embodiments FEF₂₅₋₇₅ is measured post-bronchodilation.

In some embodiments, administering dexpramipexole, or a pharmaceutically acceptable salt thereof, improves the subject's ratio of forced expiratory volume in 3 seconds to FVC (FEV₃/FVC). In some embodiments FEV₃/FVC is measured pre-bronchodilation. In some embodiments FEV₃/FVC is measured post-bronchodilation.

In some embodiments, administering dexpramipexole, or a pharmaceutically acceptable salt thereof, improves a measurement selected from the group consisting of St George's Respiratory Questionnaire for COPD Patients (SGRQ-C), COPD Assessment Test (CAT), Modified Medical Research Council Grading System (mMRC), Evaluating Respiratory Symptoms in Chronic Obstructive Pulmonary Disease (E-RS:COPD), and any combination thereof.

In some embodiments, administering dexpramipexole, or a pharmaceutically acceptable salt thereof, improves the subject's SGRQ-C total score measurement. In some embodiments the SGRQ-C measurement improves by about 1 to about 40 points. In some embodiments the SGRQ-C measurement improves by about 10 to about 15 points. In some embodiments the SGRQ-C measurement improves by about 10 to about 20 points. In some embodiments the SGRQ-C measurement improves by about 20 to about 30 points. In some embodiments the SGRQ-C measurement improves by about 20 to about 40 points. In some embodiments the SGRQ-C measurement improves by about 5 to about 10 points. In some embodiments the SGRQ-C measurement improves by about 10 to about 15 points. In some embodiments the SGRQ-C measurement improves by about 15 to about 20 points.

In some embodiments, administering dexpramipexole, or a pharmaceutically acceptable salt thereof, improves the subject's CAT total score measurement. In some embodiments the CAT measurement improves by about 1 to about 30 points. In some embodiments the CAT measurement improves by about 10 to about 15 points. In some embodiments the CAT measurement improves by about 10 to about 20 points. In some embodiments the CAT measurement improves by about 20 to about 30 points. In some embodiments the CAT measurement improves by about 5 to about 10 points. In some embodiments the CAT measurement improves by about 10 to about 15 points. In some embodiments the CAT measurement improves by about 15 to about 20 points.

In some embodiments, prior to treatment with dexpramipexole or a pharmaceutically acceptable salt thereof, the subject is already receiving a treatment regimen comprising administration of an inhaled corticosteroid (ICS), thereby treating the COPD in the subject. In some embodiments, the treatment regimen comprises administration of an ICS and a long-acting β2 agonist (LABA). In some embodiments, the treatment regimen comprises administration of an ICS and a long-acting muscarinic antagonist (LAMA). In some embodiments, the treatment regimen comprises administration of an ICS, LABA, and LAMA. In some embodiments, the subject has received the treatment regimen for at least 12 weeks. In some embodiments, the subject has received a stable dose of the treatment regimen for at least 4 weeks. In some embodiments, the ICS is selected from the group consisting of beclomethasone, fluticasone, ciclesonide, mometasone, budesonide, flunisolide, and any combination thereof. In some embodiments, the LABA is selected from the group consisting of formoterol fumarate, salmeterol, salmeterol xinafoate, arformoterol tartrate, olodaterol, vilanterol, indacaterol, and any combination thereof. In some embodiments, the LAMA is selected from the group consisting of aclidinium, glycopyrrolate, glycopyrronium, tiotropium, umeclidinium, and any combination thereof. In some embodiments, prior to treatment with dexpramipexole or a pharmaceutically acceptable salt thereof, the subject is receiving one or more COPD medications selected from the group consisting of short-acting bronchodilators, long-acting bronchodilators, ultra-long-acting bronchodilators, xanthines, short-acting muscarinic antagonists, long-acting muscarinic antagonists, leukotriene antagonists, 5-lipoxygenase inhibitors, and any combination thereof.

In some embodiments, the subject is a current or former smoker. In some embodiments, the subject's COPD is from toxic exposure.

### BRIEF DESCRIPTION OF THE DRAWINGS

FIG. 1 shows a graph of Absolute Eosinophil Count Over Time in participants treated with dexpramipexole from 37.5 to 150 mg BID.

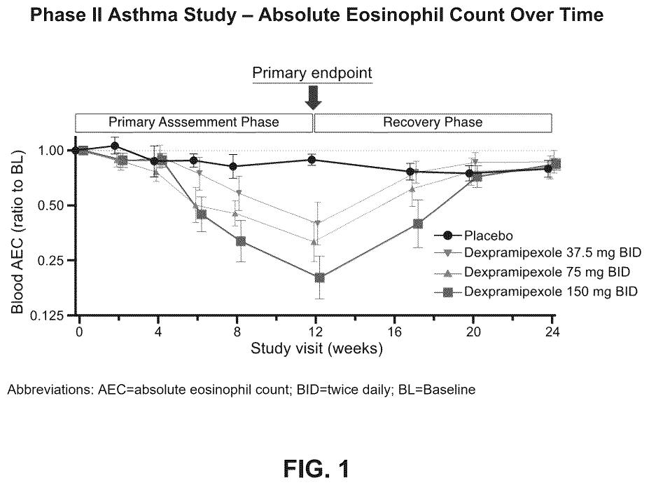

FIG. 2 depicts a schematic outline of the Phase II Clinical Trial in Participants with Eosinophilic COPD.

### DETAILED DESCRIPTION OF THE DISCLOSURE

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. In general, the headings provided herein are not to be understood as limiting the various aspects and embodiments of the present disclosure. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

### I. Definitions

In the following, some definitions are provided to further define the present disclosure. Additional definitions may be found throughout the specification.

The singular forms “a”, “an”, and “the” include plural referents unless the context clearly dictates otherwise. The terms “a” (or “an”), as well as the terms “one or more” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical value or range, it modifies that value or range by extending the boundaries above and below the numerical value(s) set forth by a variance of 10 percent, up or down (higher or lower). For example, within the meaning of the present disclosure, “about 50%” means in the range of 45%-55%, and “about 50% to about 60%” means in the range of 45%-66%.

The term “and/or” where used herein is to be taken as specific disclosure of each of the specified features or components with or without the other. Thus, the term “and/or” as used in a phrase such as “A and/or B” herein is intended to include “A and B”, “A or B”, “A” (alone), and “B” (alone). Likewise, the term “and/or” as used in a phrase such as “A, B, and/or C” is intended to encompass each of the following aspects: “A, B, and C”, “A, B, or C”, “A or C”, “A or B”, “B or C”, “A and C”, “A and B”, “B and C”, “A” (alone), “B” (alone), and “C” (alone).

The terms “administration of” and or “administering a” compound or composition should be understood to mean providing a compound or composition described herein to one or more subjects.

As used herein, the term “comprising” means “including, but not limited to.”

As used herein, the term “consisting of” means the method or composition includes only the steps or components specifically recited thereafter.

As used herein, the term “any combination thereof” or “a combination thereof” covers any combination of the components or steps recited before the term. For example, the expression “a composition further comprising A, B, C, D, or any combination thereof” includes inter alia “a composition further comprising A,” “a composition further comprising A and B,” “a composition further comprising B and D,” “a composition further comprising A, B and C,” and “a composition further comprising A, B, C, and D.”

The term “dexpramipexole,” as used herein, refers to (6R)-2-amino-4,5,6,7-tetrahydro-6-(propylamino)benzothiazole. The chemical structure of dexpramipexole is given above in formula (I). The term “dexpramipexole” refers to the “free base” (i.e., the neutral form of dexpramipexole as shown above in formula (I)) unless the context clearly dictates otherwise.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case can be determined using techniques, such as a dose escalation study.

The term “equivalent” is used herein to specify a certain amount of an ingredient with a reference to a more specific form (such as a specific salt) of the ingredient. For example, the term “dexpramipexole dihydrochloride equivalent” is used to specify the amount of dexpramipexole, or a pharmaceutically acceptable salt thereof, in a pharmaceutical composition that corresponds the same quantity as dexpramipexole dihydrochloride. For instance, an amount of 112 mg dexpramipexole corresponds to 150 mg dexpramipexole dihydrochloride equivalent, an amount of 224 mg dexpramipexole corresponds to 300 mg dexpramipexole dihydrochloride equivalent, and an amount of 56 mg dexpramipexole corresponds to 75 mg dexpramipexole dihydrochloride equivalent.

For another example, the term “dexpramipexole dihydrochloride equivalent” is used to specify the amount of dexpramipexole, or a pharmaceutically acceptable salt thereof, in a pharmaceutical composition that corresponds the same quantity as dexpramipexole dihydrochloride. For instance, an amount of 319 mg dexpramipexole dihydrochloride monohydrate corresponds to 300 mg dexpramipexole dihydrochloride equivalent, and an amount of 224 mg dexpramipexole corresponds to 300 mg dexpramipexole dihydrochloride equivalent. As another example, an amount of 160 mg dexpramipexole dihydrochloride monohydrate corresponds to 150 mg dexpramipexole dihydrochloride equivalent, and an amount of 112 mg dexpramipexole corresponds to 150 mg dexpramipexole dihydrochloride equivalent.

As used herein, the term “Once Daily” or “OD” means “once a day.”

As used herein, the term “Bis In Die” or “BID” means “twice a day.”

As used herein, the term “eosinophil” refers to an eosinophil granulocyte. In some embodiments, the term “eosinophil” refers to a human eosinophil progenitor (hEoP). In some embodiments, the term “eosinophil” refers to an eosinophil lineage-committed progenitor (EoP). In some embodiments, the term “eosinophil” refers to a human common myeloid progenitor (hCMP). In some embodiments, the term “eosinophil” refers to any combination of an eosinophil granulocyte, a human eosinophil progenitor (hEoP), an eosinophil lineage-committed progenitor (EoP), and a human common myeloid progenitor (hCMP). In some embodiments, the term “eosinophil” refers to a CD34+CD125+ progenitor cell. In some embodiments, the term “eosinophil” refers to an eosinophil residing in the bone marrow, in the systemic circulatory system, and/or in organ tissues, including the bone marrow, lungs, and airways. In some embodiments, the organ tissue is the lung, the skin, the heart, the brain, the eye, the gastrointestinal tract, the thymus, the spleen, the kidney, the bladder, the ear, the nose, the sinuses, the oral cavity, the upper respiratory tract, the bone marrow, or combinations thereof.

The term “improves” is used to convey that the present invention changes either the form, characteristics, structure, function and/or physical attributes of the tissue to which it is being provided, applied or administered. “Improves” may also refer to the overall physical state, or QoL of an individual to whom an active agent has been administered. For example, the overall physical state of an individual may “improve” if one or more symptoms of the disease, condition or disorder are alleviated by administration of an active agent.

The term “pharmaceutically acceptable salt” is meant to indicate those salts that are suitable for use in contact with the tissues of a human without undue toxicity, irritation, allergic response, and the like, and are commensurate with a reasonable benefit/risk ratio. For example, Remington's Pharmaceutical Sciences, 23^{th} ed. (2020) and Berge et al. (1977), J. Pharm. Sciences, Vol 6, 1-19 describe pharmaceutically acceptable salts in detail. Pharmaceutically acceptable salts can generally also be in the form of hydrates (such as in the form of a monohydrate). In some embodiments, the pharmaceutically acceptable salt of dexpramipexole is dexpramipexole dihydrochloride or a hydrate thereof (such as a monohydrate). Examples of suitable pharmaceutically acceptable salts of dexpramipexole within the scope of the present disclosure include, but are not limited to, dexpramipexole dihydrochloride and dexpramipexole dihydrochloride monohydrate. Further suitable “pharmaceutically acceptable salts” of dexpramipexole within the scope of the present disclosure are described throughout the specification.

The terms “subject” and “participant” are used interchangeably and encompass mammals. In some aspects, the mammal is a human.

The term “treating” refers to alleviating of the signs or symptoms associated with a specific disorder, disease, or condition, and/or removing of the signs or symptoms associated with a specific disorder, disease, or condition, and/or preventing of the worsening of the signs or symptoms associated with a specific disorder, disease, or condition. In some aspects, a treatment alleviates signs or symptoms associated with a specific disorder, disease, QoL, improve health status, or condition. In other aspects, the treatment removes signs or symptoms associated with a specific disorder, disease, or condition. In other aspects, the treatment prevents worsening of the signs or symptoms associated with a specific disorder, disease, or condition. In other aspects, the treatment reduces patient risk.

The term “preventing” refers to prophylaxis of a specific disorder, disease, or condition in a human. In certain aspects, the human may be predisposed to the specific disorder, disease, or condition but does not yet experience or display the pathology, signs, or symptoms of the specific disorder, disease, or condition.

The term “in need thereof,” as used herein, means that the human subject has a need for the particular treatment or prevention and that the treatment or prevention is being given to the subject for that particular purpose.

As used herein, “moderate COPD exacerbation” is defined as an acute exacerbation of COPD that requires either systemic corticosteroids (intramuscular [IM], intravenous, or oral) and/or antibiotics.

As used herein, “severe COPD exacerbation” is defined as an acute exacerbation of COPD that requires hospitalization (defined as inpatient admission≥24 hours in hospital, observation area, emergency department, or other equivalent health care facility, depending on the health care system) or results in death.

As used herein, “AEC Ratio—Baseline to Week 16≥0.5 (AECR≥0.5)” is defined as a subject that displays greater than or equal to a 50% decrease in blood absolute eosinophil count (AEC) in response to treatment with dexpramipexole.

As used herein, “AEC Ratio—Baseline to Week 16<0.5 (AECR<0.5)” is defined as a subject that displays less than a 50% decrease in blood absolute eosinophil count (AEC) in response to treatment with dexpramipexole.

### II. Pharmaceutical Compositions

The present disclosure provides methods of treating eosinophilic COPD, comprising orally administering to the subject a therapeutically effective amount of dexpramipexole, or a pharmaceutically acceptable salt thereof. In some aspects, the method comprises a pharmaceutical composition in the form of an orally deliverable tablet comprising dexpramipexole or a pharmaceutically acceptable salt thereof

### 1. Pharmaceutically Acceptable Salts of Dexpramipexole

Dexpramipexole can be administered as a free base or a pharmaceutical acceptable salt, such as the dihydrochloride salt. Lists of suitable salts are found in Remington's Pharmaceutical Sciences, 23rd ed., Elsevier Inc., Academic Press, Cambridge, MA, pages 307-314, 2020, which is incorporated herein by reference in its entirety.

In some embodiments, the pharmaceutically acceptable salt of dexpramipexole is an inorganic acid salt, an organic acid salt, or an amino acid salt.

In some embodiments, the pharmaceutically acceptable salt of dexpramipexole is a halogenic acid salt.

In some embodiments, the halogenic acid salt is a hydrobromic, hydrochloric, hydrofluoric, or hydroiodic acid salt.

In some embodiments, the inorganic acid salt is a nitric, perchloric, sulfuric, or phosphoric acid salt.

In some embodiments, the organic acid salt is a sulfonic, tartaric acid, acetic, malic, fumaric, succinic, citric, benzoic, gluconic, lactic, mandelic, mucic, pamoic, pantothenic, exalic or maleic acid salt. In some embodiments, the sulfonic acid salt is a methane sulfonic, trifluoromethane sulfonic, ethane sulfonic, benzene sulfonic or p-toluene sulfonic acid salt.

In some embodiments, the amino acid salt is aspartic or glutamic acid salt.

In some embodiments, the pharmaceutically acceptable salt is a methane sulfonic acid salt (“mesylate salt”), sulfuric acid salt (“sulfate salt”), tartaric acid salt (“tartrate salt”), p-toluene sulfonic acid salt (“tosylate salt”), phosphoric acid salt (“phosphate salt”), maleic acid salt (“maleate salt”), fumaric acid salt (“fumarate salt”), malic acid salt (“malate salt”), citric acid salt (“citrate salt”), succinic acid salt (“succinate salt”), or any combination thereof.

In some specific embodiments, the pharmaceutically acceptable salt is a methane sulfonic acid salt, phosphoric acid salt, fumaric acid salt, or any combination thereof.

The acid addition salt may be a mono- or di-acid addition salt, such as dihydrobromic, dihydrochloric, dihydrofluoric, dihydroiodic, disulfuric, diphosphoric, or diorganic acid salt. The acid addition salt may additionally be in the form of a hydrate, such as a dihydrochloride monohydrate.

In some aspects, the pharmaceutically acceptable salt of dexpramipexole is dexpramipexole dihydrochloride or a hydrate thereof (such as a monohydrate). In some aspects, the pharmaceutically acceptable salt of dexpramipexole is dexpramipexole dihydrochloride. In some aspects, the pharmaceutically acceptable salt of dexpramipexole is dexpramipexole dihydrochloride monohydrate.

As used herein, dexpramipexole is 99.9% to 100% enantiomerically pure. In some aspects, the dexpramipexole can have a chiral purity for dexpramipexole of at least 99.5%, at least 99.6%, at least 99.7%, at least 99.8%, at least 99.9%, at least 99.95%, or more at least 99.99%. In some aspects, the chiral purity for dexpramipexole is 100%.

All embodiments for amounts of, chiral purity of, and dosage form of dexpramipexole, or a pharmaceutically acceptable salt thereof, in the pharmaceutical compositions described herein are provided separately for the sake of brevity, but may be joined in any suitable combination.

### 2. Dosage of Dexpramipexole, or a Pharmaceutically Acceptable Salt Thereof

The amount of dexpramipexole, or a pharmaceutically acceptable salt thereof, administered to a subject according to the method of the present disclosure is, generally, a therapeutically effective amount. As used herein, the phrase “a therapeutically effective amount” means an amount of dexpramipexole, or a pharmaceutically acceptable salt thereof, that results in a significant change in one or more of the following: rate of COPD exacerbations, mucus plug score, rate of hospitalization due to exacerbations, rate of steroids and/or antibiotics use for treatment of COPD, level of biomarker associated with eosinophilic COPD and/or lung function decline, AEC, FEV₁, FVC, FEV₁/FVC, FEF₂₅₋₇₅, FEV₃/FVC, SGRQ-C, CAT, mMRC, E-RS:COPD.

In some aspects, the method comprises orally administering a daily dose of about 75 mg to about 300 mg of dexpramipexole, or a pharmaceutically acceptable salt thereof. In some aspects, the method comprises orally administering a daily dose of about 75 mg to about 250 mg of dexpramipexole, or a pharmaceutically acceptable salt thereof. In some aspects, the method comprises orally administering a daily dose of about 75 mg to about 200 mg of dexpramipexole, or a pharmaceutically acceptable salt thereof. In some aspects, the method comprises orally administering a daily dose of about 75 mg to about 150 mg of dexpramipexole, or a pharmaceutically acceptable salt thereof. In some aspects, the method comprises orally administering a daily dose of about 150 mg to about 300 mg of dexpramipexole, or a pharmaceutically acceptable salt thereof. In some aspects, the method comprises orally administering a daily dose of about 150 mg to about 200 mg of dexpramipexole, or a pharmaceutically acceptable salt thereof. In some aspects, the method comprises orally administering a daily dose of about 150 mg to about 250 mg of dexpramipexole, or a pharmaceutically acceptable salt thereof.

In some aspects, the daily dose of dexpramipexole, or a pharmaceutically acceptable salt thereof, is about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg.

In some aspects, the amount of dexpramipexole, or a pharmaceutically acceptable salt thereof, is about 75 mg of dexpramipexole dihydrochloride equivalent. In some aspects, the amount of dexpramipexole, or a pharmaceutically acceptable salt thereof, is about 150 mg of dexpramipexole dihydrochloride equivalent. In some aspects, the amount of dexpramipexole, or a pharmaceutically acceptable salt thereof, is about 300 mg of dexpramipexole dihydrochloride equivalent.

### 3. Administration of Dexpramipexole, or a Pharmaceutically Acceptable Salt Thereof

Dexpramipexole, or a pharmaceutically acceptable salt thereof, can be in the form of an orally deliverable tablet that is suitable for daily administration to a human.

The term “orally deliverable,” as used herein, means that a corresponding pharmaceutical composition (such as a tablet) is suitable for oral administration to a human by swallowing the composition as a whole. The term “orally administering” as used herein may include the act of self-administration by a human in need thereof or administration by another person such as a health care provider.

Dexpramipexole, or a pharmaceutically acceptable salt thereof, can be administered according to varying schedules. In some aspects, the mode of administration can be continuous. In some embodiments, the mode of administration is regular and/or long-term. For example, the pharmaceutical formulations can be administered once daily, twice daily, or more, as specified by a physician. In some aspects, dexpramipexole, or a pharmaceutically acceptable salt thereof, can be administered once daily or twice daily. In one aspect, dexpramipexole, or a pharmaceutically acceptable salt thereof, can be administered once daily. In another aspect, dexpramipexole, or a pharmaceutically acceptable salt thereof, can be administered twice daily.

In one aspect, dexpramipexole, or a pharmaceutically acceptable salt thereof, can be administered for about 1 month. In one aspect, dexpramipexole, or a pharmaceutically acceptable salt thereof, can be administered for about 2 months. In one aspect, dexpramipexole, or a pharmaceutically acceptable salt thereof, can be administered for about 3 months. In one aspect, dexpramipexole, or a pharmaceutically acceptable salt thereof, can be administered for about 6 months. In one aspect, dexpramipexole, or a pharmaceutically acceptable salt thereof, can be administered for about 1 year. In one aspect, dexpramipexole, or a pharmaceutically acceptable salt thereof, can be administered longer than 1 year. In one aspect, dexpramipexole, or a pharmaceutically acceptable salt thereof, can be administered as an episodic treatment. In one aspect, dexpramipexole, or a pharmaceutically acceptable salt thereof, can be administered as a maintenance treatment.

In some aspects, dexpramipexole, or a pharmaceutically acceptable salt thereof, can be administered as about 75 mg, about 150 mg, or about 300 mg once per day.

In some aspects, dexpramipexole, or a pharmaceutically acceptable salt thereof, can be administered as 75 mg, 150 mg, or 300 mg once per day.

In some aspects, dexpramipexole, or a pharmaceutically acceptable salt thereof, can be administered as about 37.5 mg, about 75 mg, or about 150 mg twice per day.

In some aspects, dexpramipexole, or a pharmaceutically acceptable salt thereof, can be administered as 37.5 mg, 75 mg, or 150 mg twice per day.

Dexpramipexole, or a pharmaceutically acceptable salt thereof, can be administered for any total length of time that provides an effective therapeutic benefit. In some aspects dexpramipexole, or a pharmaceutically acceptable salt thereof, can be administered over the course of a period of days, weeks, months, or years. For example, dexpramipexole, or a pharmaceutically acceptable salt thereof, can be administered once or twice daily for at least 1, 2, 3, 4, 5, 6, or 7 days (i.e., about 1 week); about 2 weeks, about 3 weeks, or about 4 weeks; about 2 months, about 3 months, about 4 months, about 5 months, about 6 months, about 7 months, about 8 months, about 9 months, about 10 months, about 11 months, or about 12 months (i.e., about a year); or more, as specified by a physician.

### III. Methods of Treatment or Prevention

Provided herein are methods of treating eosinophilic COPD and/or inflammatory responses of eosinophilic COPD in a human subject with eosinophilic COPD, comprising orally administering to the subject a therapeutically effective amount of dexpramipexole, or a pharmaceutically acceptable salt thereof.

Patients with COPD commonly present with dyspnea, chronic cough, and/or sputum production, and occasionally wheezing. Unfortunately, delayed diagnosis is common and many patients present only after they have experienced≥1 exacerbation, often mistakenly labeled as recurrent bronchitis. Early diagnosis and treatment are essential to improve patients' lung function, functional status, and QoL, and to reduce exacerbations.

Spirometry is essential and required to confirm a COPD diagnosis. It also is useful for tracking treatment response, potentially adjusting medications, and monitoring disease progression. When rapid disease progression is identified, further evaluation and referral to a lung specialist are indicated. A postbronchodilator (15-30 minutes after 2-4 puffs of a short-acting bronchodilator) forced expiratory volume in 1 second (FEV₁) to forced vital capacity (FVC) ratio of <0.70 confirms the presence of persistent or fixed airflow limitation. FVC is the maximal volume of air that can be forcibly exhaled after taking in the deepest breath possible, and FEV₁ is the maximal volume of air exhaled in the first second during an FVC maneuver.

Not wishing to be bound by theory, inflammation is an important component in the pathogenesis of COPD. IL-5 is the major cytokine responsible for the growth, proliferation, differentiation, recruitment, activation, and survival of eosinophils. The pathogenesis of eosinophilic COPD also includes the development of mucus plugs in the subject's airways. The pathologic composition of mucus plugs can be diverse and results in occlusion of airways which can result in reduced airflow/air trapping or complete airway blockage leading to collapsed air sacs. This condition can lead to a decrease in forced vital capacity (FVC) and residual volume to total lung capacity ratio (RV/TLC). Individual mucus plugs can be imaged utilizing lung CT scans and the extent of mucus plug activity in the lung can be quantified utilizing a validated mucus plug score algorithm.

Meta-analyses suggest absolute eosinophils count (AEC) in the blood may correlate positively with an increased likelihood of exacerbation reduction and treatment response in COPD. Generally, the response was seen when eosinophil levels were 0.10×10⁹/L-0.30×10⁹/L (or higher).

In some embodiments, the subject has a blood AEC of ≥0.15×10⁹/L before administration of dexpramipexole, or a pharmaceutically acceptable salt thereof. In some embodiments, the subject has a blood AEC of between about 0.15×10⁹/L to about 0.30×10⁹/L before administration of dexpramipexole, or a pharmaceutically acceptable salt thereof. For example, the subject can have a blood AEC of about 0.15×10⁹/L, about 0.16×10⁹/L, about 0.17×10⁹/L, about 0.18×10⁹/L, about 0.19×10⁹/L, about 0.20×10⁹/L, about 0.22×10⁹/L, about 0.24×10⁹/L, about 0.26×10⁹/L, about 0.28×10⁹/L, or about 0.30×10⁹/L, about 0.32×10⁹/L, about 0.34×10⁹/L, about 0.36×10⁹/L, about 0.38×10⁹/L, or about 0.40×10⁹/L before administration of dexpramipexole, or a pharmaceutically acceptable salt thereof. In some embodiments, the subject has a blood AEC of ≥0.30×10⁹/L before administration of dexpramipexole, or a pharmaceutically acceptable salt thereof. In some embodiments, the subject has a blood AEC of between about 0.30×10⁹/L to about 0.5×10⁹/L before administration of dexpramipexole, or a pharmaceutically acceptable salt thereof. For example, the subject can have a blood AEC of about 0.31×10⁹/L, about 0.32×10⁹/L, about 0.33×10⁹/L, about 0.34×10⁹/L, about 0.35×10⁹/L, about 0.36×10⁹/L, about 0.37×10⁹/L, about 0.38×10⁹/L, about 0.39×10⁹/L, about 0.40×10⁹/L, about 0.41×10⁹/L, about 0.42×10⁹/L, about 0.43×10⁹/L, about 0.44×10⁹/L, about 0.45×10⁹/L, about 0.46×10⁹/L, about 0.47×10⁹/L, about 0.48×10⁹/L, about 0.49×10⁹/L, or about 0.50×10⁹/L before administration of dexpramipexole, or a pharmaceutically acceptable salt thereof. In some embodiments, the subject has a blood AEC of between about 0.50×10⁹/L to about 1.0×10⁹/L before administration of dexpramipexole, or a pharmaceutically acceptable salt thereof. For example, the subject can have a blood AEC of about 0.50×10⁹/L, about 0.55×10⁹/L, about 0.60×10⁹/L, about 0.65×10⁹/L, about 0.70×10⁹/L, about 0.75×10⁹/L, about 0.80×10⁹/L, about 0.85×10⁹/L, about 0.90×10⁹/L, about 0.95×10⁹/L, or about 1.0×10⁹/L before administration of dexpramipexole, or a pharmaceutically acceptable salt thereof. In some embodiments, the subject has a blood AEC of between about 1.0×10⁹/L to about 1.5×10⁹/L before administration of dexpramipexole, or a pharmaceutically acceptable salt thereof. For example, the subject can have a blood AEC of about 1.0×10⁹/L, about 1.1×10⁹/L, about 1.2×10⁹/L, about 1.3×10⁹/L, about 1.4×10⁹/L, or about 1.5×10⁹/L before administration of dexpramipexole, or a pharmaceutically acceptable salt thereof.

Currently, there are several recommended classes of therapy for COPD, of which bronchodilators (including β2-agonists and anticholinergics) are the mainstay of symptom management in mild and moderate disease, prescribed on an as-needed basis for mild COPD and as a maintenance therapy for moderate COPD. For the treatment of more severe COPD, guidelines recommend the addition of inhaled corticosteroids to long-acting bronchodilator therapy. Combinations of therapies have been investigated with a view to their complementary modes of action enabling multiple components of the disease to be addressed. Data from recent clinical trials indicate that triple therapy, combining an anticholinergic with an ICS and a long-acting β2-agonist (LABA), may provide clinical benefits additional to those associated with each treatment alone in patients with moderate to severe COPD. Despite receiving combination therapies, patients with COPD may continue to have exacerbations.

In some embodiments, prior to treatment with dexpramipexole or a pharmaceutically acceptable salt thereof, the subject is already receiving a treatment regimen comprising administration of an ICS, thereby treating the COPD in the subject.

In some embodiments, the treatment regimen comprises administration of an ICS and a long-acting β2 agonist (LABA)

In some embodiments, the treatment regimen comprises administration of an ICS and a long-acting muscarinic antagonist (LAMA).

In some embodiments, the treatment regimen comprises administration of an ICS, LABA, and LAMA.

In some embodiments, the subject has received the treatment regimen for at least 12 weeks. In some embodiments, the subject has received a stable dose of the treatment regimen for at least 4 weeks.

In some embodiments, the ICS is selected from the group consisting of beclomethasone, fluticasone, ciclesonide, mometasone, budesonide, flunisolide, and combinations thereof. In some embodiments, the fluticasone is fluticasone propionate or fluticasone furoate. The inhaled corticosteroids may be low-high dose inhaled corticosteroids, medium-dose ICS, or high-dose ICS.

In some embodiments, the LABA is selected from the group consisting of formoterol fumarate, salmeterol, salmeterol xinafoate, arformoterol tartrate, olodaterol, vilanterol, indacaterol, and combinations thereof.

In some embodiments, the LAMA is selected from the group consisting of aclidinium, glycopyrrolate, glycopyrronium, tiotropium, umeclidinium, and any combination thereof.

In some embodiments, prior to treatment with dexpramipexole or a pharmaceutically acceptable salt thereof, the subject is receiving one or more COPD medications selected from the group consisting of short-acting bronchodilators, long-acting bronchodilators, ultra-long-acting bronchodilators, xanthines, short-acting muscarinic antagonists, long-acting muscarinic antagonists, leukotriene antagonists, 5-lipoxygenase inhibitors, and any combination thereof.

Most often COPD can be prevented by reducing risk factors including tobacco smoke, occupational dusts and chemicals, biomass fuel, and air pollution. In some embodiments, the subject is a current or former smoker. In some embodiments, the subject's COPD is from toxic exposure(s).

In some embodiments, the subject is 18 years of age or older. In some embodiments, the subject is 40 years or older. In some embodiments, the subject is between the ages of 18 to 75 years old. In some embodiments, the subject is about 18 to about 85 years old. In some embodiments, the subject is about 18 to about 100 years old. In some embodiments, the subject is about 25 to about 85 years old. In some embodiments, the subject is about 30 to about 85 years old. In some embodiments, the subject is about 35 to about 85 years old. In some embodiments, the subject is about 40 to about 85 years old. In some embodiments, the subject is about 45 to about 85 years old. In some embodiments, the subject is about 50 to about 85 years old. In some embodiments, the subject is about 55 to about 85 years old. In some embodiments, the subject is about 60 to about 85 years old. In some embodiments, the subject is about 65 to about 85 years old. In some embodiments, the subject is about 70 to about 85 years old. In some embodiments, the subject is about 75 to about 85 years old. In some embodiments, the subject is about 80 to about 85 years old. In some embodiments, the subject is about 85 to about 100 years old.

Mucus dysfunction, a central pathology in patients with COPD, is characterized by excess mucus production, hypersecretion, and reduced clearance, leading to accumulation in the airways as mucus plugs. Computed tomography (CT) scans of individuals with COPD can be assessed by a physician or clinician trained in lung imaging. Mucus plug scoring is assigned based on the number of lung segments with mucus plugs, ranging from 0 (no mucus plugs seen on CT scan) to 20 (all lung segments with mucus plugs).

In some embodiments, the subject has evidence of mucus plugs (i.e., a mucus plug score of ≥1) before administration of dexpramipexole, or a pharmaceutically acceptable salt thereof. In some embodiments, the subject has a mucus plug score of 1, 2, 3, 4, 5, 6, 7, 8, 9, 10, 11, 12, 13, 14, 15, 16, 17, 18, 19, or 20 before administration of dexpramipexole, or a pharmaceutically acceptable salt thereof. In some embodiments, the subject has a mucus plug score of 0 before administration of dexpramipexole, or a pharmaceutically acceptable salt thereof. In some embodiments, administering dexpramipexole, or a pharmaceutically acceptable salt thereof, reduces the presence of mucus plugs as demonstrated by reducing the mucus plug score.

Provided herein are methods of reducing the presence of mucus plugs as demonstrated by reducing a mucus plug score in a human subject with eosinophilic COPD, comprising orally administering to the subject a therapeutically effective amount of dexpramipexole, or a pharmaceutically acceptable salt thereof.

In some embodiments, administering dexpramipexole, or a pharmaceutically acceptable salt thereof, reduces the presence of mucus plugs as demonstrated by reducing the mucus plug score. In some embodiments, the subject's mucus plug score is reduced by ≥1 after administration of dexpramipexole, or a pharmaceutically acceptable salt thereof. In some embodiments, the subject's mucus plug score is reduced by 1, 2, 3, 4, 5, 6, 7, 8, 9, 10, 11, 12, 13, 14, 15, 16, 17, 18, 19, or 20 after administration of dexpramipexole, or a pharmaceutically acceptable salt thereof.

CT scans of individuals with COPD can also be assessed for a number of outcomes including but not limited to airway lumen area, airway wall area, particle deposition, lung volumes, low attentuation areas (%<−856HU, %<−9501U), DPM air trapping, mucus plug score, airway wall thickness, computational airflow analysis, and vascular analysis of blood vessels in the lung. In some embodiments, administering dexpramipexole, or a pharmaceutically acceptable salt thereof, improves the subject's lung function. In some embodiments, administering dexpramipexole, or a pharmaceutically acceptable salt thereof, improves the subject's CT scan biomarkers. In some embodiments, administering dexpramipexole, or a pharmaceutically acceptable salt thereof, improves the subject's lung function and the subject's CT scan biomarkers. Examples of CT scan biomarkers include, but are not limited to, airway remodeling biomarkers (e.g., airway lumen area, airway wall thickness, airway wall area, airway wall area fraction, Pi10, mucus plug score, mucus plug distribution, and mucus plug burden), tissue remodeling biomarkers (e.g., total lung volume, total air volume, total tissue volume, low attenuation area, 15^{th} percentile, skewness, kurtosis, and mean lung density), functional biomarkers (e.g., air trapping: −8561HU, air trapping: ventilation defect probability mapping, jacobian, and anisotropic deformation index), and vascular remodeling biomarkers (e.g., total vessel volume, total peripheral vessel volume, and peripheral vessel fraction).

A COPD exacerbation is defined as an acute worsening of respiratory symptoms that results in additional therapy, and is mainly triggered by respiratory infections (viral and bacterial) and environmental factors such as toxic exposure.

Current treatment goals for exacerbations are to minimize the negative impact of the current exacerbation and reduce the risk of any future exacerbations. The majority of patients that experience exacerbations can be managed on an outpatient basis with pharmacologic therapies (moderate COPD exacerbations); however, some patients may require hospitalization (severe COPD exacerbations).

Many novel treatments have failed to prevent exacerbations. However, blood eosinophil counts can help healthcare professionals to predict probability of clinical benefit and reduction of exacerbations.

Provided herein are methods of reducing the rate of COPD exacerbations in a human subject with eosinophilic COPD, comprising orally administering to the subject a therapeutically effective amount of dexpramipexole, or a pharmaceutically acceptable salt thereof.

Pulmonary function is measured by performing a spirometry, a physiological test that measures a number of indices based on the maximal amount of air that an individual can exhale or inhale and includes forced expiratory volume in 1 second (FEV₁), the volume of air moved in the first second of exhalation, which is a well-accepted measure of respiratory disorders. Forced vital capacity (FVC) is the total volume of air exhaled during the entire forced expiratory volume (FEV) test. The FEV₁/FVC ratio is a calculated measure that may provide a more specific measure of bronchoconstriction, independent of processes such as air trapping. Pulmonary function testing can be tested pre-bronchodilator (after waiting a sufficient time so that all bronchodilator medications have cleared) or post-bronchodilator, typically 15-30 minutes after inhaling a short-acting bronchodilator, such as albuterol. Bronchodilator reversibility (BDR) is calculated and assessed by analyzing FEV₁ pre- and post-bronchodilator, and calculating the change both as absolute (ml) and percentage change.

In some embodiments, administering dexpramipexole, or a pharmaceutically acceptable salt thereof, improves the subject's mean FEV₁. In some embodiments, the subject's FEV₁ is increased by about 5% to about 50%. In some embodiments, the subject's FEV₁ is increased by about 5% to about 10%. In some embodiments, the subject's FEV₁ is increased by about 10% to about 30%. In some embodiments, the subject's FEV₁ is increased by about 30% to about 50%. In some embodiments, the subject's FEV₁ is increased by about 15% to about 50%. In some embodiments, the subject's FEV₁ is increased by about 20% to about 50%. In some embodiments, the subject's FEV₁ is increased by about 25% to about 50%. In some embodiments, the subject's FEV₁ is increased by about 30% to about 50%. In some embodiments, the subject's FEV₁ is increased by about 35% to about 50%. In some embodiments, the subject's FEV₁ is increased by about 40% to about 50%. In some embodiments, the subject's FEV₁ is increased by about 55% to about 50%. In some embodiments, the subject's FEV₁ is increased by about 15% to about 45%. In some embodiments, the subject's FEV₁ is increased by about 15% to about 40%. In some embodiments, the subject's FEV₁ is increased by about 15% to about 35%. In some embodiments, the subject's FEV₁ is increased by about 5% to about 30%. In some embodiments, the subject's FEV₁ is increased by about 15% to about 25%. In some embodiments, the subject's FEV₁ is increased by about 15% to about 20%. In some embodiments, the subject's FEV₁ is increased by about 5%, about 10%, about 15%, about 20%, about 25%, about 30%, about 35%, about 40%, about 45%, or about 50%. In some embodiments, the subject's FEV₁ is increased by about 16% to about 30%. In some embodiments, the subject's FEV₁ is increased by about 18% to about 30%. In some embodiments, the subject's FEV₁ is increased by about 20% to about 30%. In some embodiments, the subject's FEV₁ is increased by about 20% to about 30%. In some embodiments, the subject's FEV₁ is increased by about 22% to about 30%. In some embodiments, the subject's FEV₁ is increased by about 24% to about 30%. In some embodiments, the subject's FEV₁ is increased by about 26% to about 30%. In some embodiments, the subject's FEV₁ is increased by about 28% to about 30%. In some embodiments, the subject's FEV₁ is increased by about 30% to about 75%. In some embodiments, the subject's FEV₁ is increased by about 35% to about 75%. In some embodiments, the subject's FEV₁ is increased by about 40% to about 75%. In some embodiments, the subject's FEV₁ is increased by about 45% to about 75%. In some embodiments, the subject's FEV₁ is increased by about 55% to about 75%. In some embodiments, the subject's FEV₁ is increased by about 65% to about 75%. In some embodiments FEV₁ is measured pre-bronchodilation. In some embodiments FEV₁ is measured post-bronchodilation.

In some embodiments, the subject's FEV₁ is increased by about 30 ml to about 1000 ml. In some embodiments, the subject's FEV₁ is increased by about 30 ml to about 900 ml. In some embodiments, the subject's FEV₁ is increased by about 30 ml to about 800 ml. In some embodiments, the subject's FEV₁ is increased by about 30 ml to about 700 ml. In some embodiments, the subject's FEV₁ is increased by about 30 ml to about 600 ml. In some embodiments, the subject's FEV₁ is increased by about 30 ml to about 500 ml. In some embodiments, the subject's FEV₁ is increased by about 30 ml to about 400 ml. In some embodiments, the subject's FEV₁ is increased by about 30 ml to about 300 ml. In some embodiments, the subject's FEV₁ is increased by about 30 ml to about 200 ml. In some embodiments, the subject's FEV₁ is increased by about 30 ml to about 100 ml. In some embodiments, the subject's FEV₁ is increased by about 100 ml to about 1000 ml. In some embodiments, the subject's FEV₁ is increased by about 200 ml to about 1000 ml. In some embodiments, the subject's FEV₁ is increased by about 300 ml to about 1000 ml. In some embodiments, the subject's FEV₁ is increased by about 400 ml to about 1000 ml. In some embodiments, the subject's FEV₁ is increased by about 500 ml to about 1000 ml. In some embodiments, the subject's FEV₁ is increased by about 600 ml to about 1000 ml. In some embodiments, the subject's FEV₁ is increased by about 700 ml to about 1000 ml. In some embodiments, the subject's FEV₁ is increased by about 800 ml to about 1000 ml. In some embodiments, the subject's FEV₁ is increased by about 900 ml to about 1000 ml. For example, the subject's FEV₁ can be increased by about 30 ml, about 50 ml, about 100 ml, about 150 ml, about 200 ml, about 250 ml, about 300 ml, about 350 ml, about 400 ml, about 450 ml, about 500 ml, about 550 ml, about 600 ml, about 650 ml, about 700 ml, about 750 ml, about 800 ml, about 850 ml, about 900 ml, about 950 ml, or about 1000 ml. In some embodiments, the subject's FEV₁ is increased by about 40 ml to about 500 ml. In some embodiments, the subject's FEV₁ is increased by about 50 ml to about 150 ml. For example, the subject's FEV₁ is increased by about 100 ml. In some embodiments, the subject's FEV₁ is increased by about 200 mL to about 500 mL after administration. In some embodiments, the subject's FEV₁ is increased by about 250 mL to about 500 mL after administration. In some embodiments, the subject's FEV₁ is increased by about 300 mL to about 500 mL after administration. In some embodiments, the subject's FEV₁ is increased by about 350 mL to about 500 mL after administration. In some embodiments, the subject's FEV₁ is increased by about 400 mL to about 500 mL after administration. In some embodiments, the subject's FEV₁ is increased by about 450 mL to about 500 mL after administration. In some embodiments, the subject's FEV₁ is increased by about 500 mL to about 900 mL. In some embodiments, the subject's FEV₁ is increased by about 550 mL to about 900 mL. In some embodiments, the subject's FEV₁ is increased by about 600 mL to about 900 mL. In some embodiments, the subject's FEV₁ is increased by about 650 mL to about 900 mL. In some embodiments, the subject's FEV₁ is increased by about 700 mL to about 900 mL. In some embodiments, the subject's FEV₁ is increased by about 750 mL to about 900 mL. In some embodiments, the subject's FEV₁ is increased by about 800 mL to about 900 mL. In some embodiments, the subject's FEV₁ is increased by about 850 mL to about 900 mL. In some embodiments FEV₁ is measured pre-bronchodilation. In some embodiments FEV₁ is measured post-bronchodilation.

In some embodiments, administering dexpramipexole, or a pharmaceutically acceptable salt thereof, improves the subject's FVC. In some embodiments, the subject's FVC is increased by about 5% to about 50%. In some embodiments, the subject's FVC is increased by about 10% to about 50%. In some embodiments, the subject's FVC is increased by about 15% to about 50%. In some embodiments, the subject's FVC is increased by about 20% to about 50%. In some embodiments, the subject's FVC is increased by about 25% to about 50%. In some embodiments, the subject's FVC is increased by about 30% to about 50%. In some embodiments, the subject's FVC is increased by about 35% to about 50%. In some embodiments, the subject's FVC is increased by about 40% to about 50%. In some embodiments, the subject's FVC is increased by about 45% to about 50%. In some embodiments, the subject's FVC is increased by about 5% to about 45%. In some embodiments, the subject's FVC is increased by about 5% to about 40%. In some embodiments, the subject's FVC is increased by about 5% to about 35%. In some embodiments, the subject's FVC is increased by about 5% to about 30%. In some embodiments, the subject's FVC is increased by about 5% to about 25%. In some embodiments, the subject's FVC is increased by about 5% to about 20%. In some embodiments, the subject's FVC is increased by about 5% to about 15%. In some embodiments, the subject's FVC is increased by about 5% to about 10%. In some embodiments, the subject's FVC is increased by about 5%, about 10%, about 15%, about 20%, about 25%, about 30%, about 35%, about 40%, about 45%, or about 50%. In some embodiments, the subject's FVC is increased by about 6% to about 20%. In some embodiments, the subject's FVC is increased by about 8% to about 20%. In some embodiments, the subject's FVC is increased by about 10% to about 20%. In some embodiments, the subject's FVC is increased by about 12% to about 20%. In some embodiments, the subject's FVC is increased by about 14% to about 20%. In some embodiments, the subject's FVC is increased by about 16% to about 20%. In some embodiments, the subject's FVC is increased by about 18% to about 20%. In some embodiments, the subject's FVC is increased by about 10% to about 40%. In some embodiments, the subject's FVC is increased by about 15% to about 40%. In some embodiments, the subject's FVC is increased by about 20% to about 40%. In some embodiments, the subject's FVC is increased by about 25% to about 40%. In some embodiments, the subject's FVC is increased by about 30% to about 40%. In some embodiments, the subject's FVC is increased by about 35% to about 40%. In some embodiments FVC is measured pre-bronchodilation. In some embodiments FVC is measured post-bronchodilation.

In some embodiments, the subject's FVC is increased by about 30 ml to about 1000 ml after administration. In some embodiments, the subject's FVC is increased by about 30 ml to about 1000 ml after administration. In some embodiments, the subject's FVC is increased by about 30 ml to about 900 ml after administration. In some embodiments, the subject's FVC is increased by about 30 ml to about 800 ml after administration. In some embodiments, the subject's FVC is increased by about 30 ml to about 700 ml after administration. In some embodiments, the subject's FVC is increased by about 30 ml to about 600 ml after administration. In some embodiments, the subject's FVC is increased by about 30 ml to about 500 ml after administration. In some embodiments, the subject's FVC is increased by about 30 ml to about 400 ml after administration. In some embodiments, the subject's FVC is increased by about 30 ml to about 300 ml after administration. In some embodiments, the subject's FVC is increased by about 30 ml to about 200 ml after administration. In some embodiments, the subject's FVC is increased by about 30 ml to about 100 ml after administration. In some embodiments, the subject's FVC is increased by about 100 ml to about 1000 ml after administration. In some embodiments, the subject's FVC is increased by about 200 ml to about 1000 ml after administration. In some embodiments, the subject's FVC is increased by about 300 ml to about 1000 ml after administration. In some embodiments, the subject's FVC is increased by about 400 ml to about 1000 ml after administration. In some embodiments, the subject's FVC is increased by about 500 ml to about 1000 ml after administration. In some embodiments, the subject's FVC is increased by about 600 ml to about 1000 ml after administration. In some embodiments, the subject's FVC is increased by about 700 ml to about 1000 ml after administration. In some embodiments, the subject's FVC is increased by about 800 ml to about 1000 ml after administration. In some embodiments, the subject's FVC is increased by about 900 ml to about 1000 ml after administration. For example, the subject's FVC can be increased by about 30 ml, about 50 ml, about 100 ml, about 150 ml, about 200 ml, about 250 ml, about 300 ml, about 350 ml, about 400 ml, about 450 ml, about 500 ml, about 550 ml, about 600 ml, about 650 ml, about 700 ml, about 750 ml, about 800 ml, about 850 ml, about 900 ml, about 950 ml, or about 1000 ml. In some embodiments, the subject's FVC is increased by about 40 ml to about 500 ml. In some embodiments, the subject's FVC is increased by about 50 ml to about 150 ml. For example, the subject's FVC is increased by about 100 ml. In some embodiments, the subject's FVC is increased by about 200 mL to about 500 mL after administration. In some embodiments, the subject's FVC is increased by about 250 mL to about 500 mL after administration. In some embodiments, the subject's FVC is increased by about 300 mL to about 500 mL after administration. In some embodiments, the subject's FVC is increased by about 350 mL to about 500 mL after administration. In some embodiments, the subject's FVC is increased by about 400 mL to about 500 mL after administration. In some embodiments, the subject's FVC is increased by about 450 mL to about 500 mL after administration. In some embodiments, the subject's FVC is increased by about 500 mL to about 900 mL. In some embodiments, the subject's FVC is increased by about 550 mL to about 900 mL. In some embodiments, the subject's FVC is increased by about 600 mL to about 900 mL. In some embodiments, the subject's FVC is increased by about 650 mL to about 900 mL. In some embodiments, the subject's FVC is increased by about 700 mL to about 900 mL. In some embodiments, the subject's FVC is increased by about 750 mL to about 900 mL. In some embodiments, the subject's FVC is increased by about 800 mL to about 900 mL. In some embodiments, the subject's FVC is increased by about 850 mL to about 900 mL. In some embodiments FVC is measured pre-bronchodilation. In some embodiments FVC is measured post-bronchodilation.

In some embodiments, administering dexpramipexole, or a pharmaceutically acceptable salt thereof, improves the subject's Forced Expiratory Flow 25-75% (FEF₂₅₋₇₅). In some embodiments, the subject's FEF₂₅₋₇₅ is increased by about 5% to about 50%. In some embodiments, the subject's FEF₂₅₋₇₅ is increased by about 10% to about 50%. In some embodiments, the subject's FEF₂₅₋₇₅ is increased by about 15% to about 50%. In some embodiments, the subject's FEF₂₅₋₇₅ is increased by about 20% to about 50%. In some embodiments, the subject's FEF₂₅₋₇₅ is increased by about 25% to about 50%. In some embodiments, the subject's FEF₂₅₋₇₅ is increased by about 30% to about 50%. In some embodiments, the subject's FEF₂₅₋₇₅ is increased by about 35% to about 50%. In some embodiments, the subject's FEF₂₅₋₇₅ is increased by about 40% to about 50%. In some embodiments, the subject's FEF₂₅₋₇₅ is increased by about 5% to about 45%. In some embodiments, the subject's FEF₂₅₋₇₅ is increased by about 5% to about 40%. In some embodiments, the subject's FEF₂₅₋₇₅ is increased by about 5% to about 35%. In some embodiments, the subject's FEF₂₅₋₇₅ is increased by about 5% to about 30%. In some embodiments, the subject's FEF₂₅₋₇₅ is increased by about 5% to about 25%. In some embodiments, the subject's FEF₂₅₋₇₅ is increased by about 5% to about 20%. In some embodiments, the subject's FEF₂₅₋₇₅ is increased by about 5% to about 15%. In some embodiments, the subject's FEF₂₅₋₇₅ is increased by about 5% to about 10%. In some embodiments, the subject's FEF₂₅₋₇₅ is increased by about 5%, about 10%, about 15%, about 20%, about 25%, about 30%, about 35%, about 40%, about 45%, or about 50%. In some embodiments FEF₂₅₋₇₅ is increased by about 50% to about 80%. In some embodiments FEF₂₅₋₇₅ is increased by about 50% to about 100%. In some embodiments FEF₂₅₋₇₅ is measured pre-bronchodilation. In some embodiments FEF₂₅₋₇₅ is measured post-bronchodilation.

In some embodiments, administering dexpramipexole, or a pharmaceutically acceptable salt thereof, improves the subject's Peak Flow. In some embodiments, the subject's Peak Flow is increased by about 5% to about 50%. In some embodiments, the subject's Peak Flow is increased by about 10% to about 50%. In some embodiments, the subject's Peak Flow is increased by about 15% to about 50%. In some embodiments, the subject's Peak Flow is increased by about 20% to about 50%. In some embodiments, the subject's Peak Flow is increased by about 25% to about 50%. In some embodiments, the subject's Peak Flow is increased by about 30% to about 50%. In some embodiments, the subject's Peak Flow is increased by about 35% to about 50%. In some embodiments, the subject's Peak Flow is increased by about 40% to about 50%. In some embodiments, the subject's Peak Flow is increased by about 5% to about 45%. In some embodiments, the subject's Peak Flow is increased by about 5% to about 40%. In some embodiments, the subject's Peak Flow is increased by about 5% to about 35%. In some embodiments, the subject's Peak Flow is increased by about 5% to about 30%. In some embodiments, the subject's Peak Flow is increased by about 5% to about 25%. In some embodiments, the subject's Peak Flow is increased by about 5% to about 20%. In some embodiments, the subject's Peak Flow is increased by about 5% to about 15%. In some embodiments, the subject's Peak Flow is increased by about 5% to about 10%. In some embodiments, the subject's Peak Flow is increased by about 5%, about 10%, about 15%, about 20%, about 25%, about 30%, about 35%, about 40%, about 45%, or about 50%. In some embodiments, the subject's Peak Flow is increased by about 50% to about 120%. In some embodiments, the subject's Peak Flow is increased by about 80% to about 120%. In some embodiments Peak Flow is measured pre-bronchodilation. In some embodiments Peak Flow is measured post-bronchodilation.

In some embodiments, administering dexpramipexole, or a pharmaceutically acceptable salt thereof, improves the subject's FEV₁/FVC. In some embodiments, the subject's FEV₁/FVC is increased by about 0.1 or greater. In some embodiments, the subject's FEV₁/FVC is increased by about 0.2 or greater. In some embodiments FEV₁/FVC is measured pre-bronchodilation. In some embodiments FEV₁/FVC is measured post-bronchodilation.

Among the various measurements collected during conventional spirometry, forced expiratory flow at 25% and 75% of the pulmonary volume (FEF₂₅₋₇₅) measures the average flow rates of medium-to-small airways during the FVC segment. Another test, FEV₃/FVC, refers to the fraction of the FVC that had not been expired during the first 3 seconds of the FVC.

In some embodiments, administering dexpramipexole, or a pharmaceutically acceptable salt thereof, improves the subject's FEF₂₅₋₇₅. In some embodiments FEF₂₅₋₇₅ is measured pre-bronchodilation. In some embodiments FEF₂₅₋₇₅ is measured post-bronchodilation. In some embodiments, administering dexpramipexole, or a pharmaceutically acceptable salt thereof, improves the subject's FEV₃/FVC. In some embodiments FEV₃/FVC is measured pre-bronchodilation. In some embodiments FEV₁/FVC is measured post-bronchodilation.

A severe exacerbation is defined as an acute exacerbation of COPD that requires hospitalization (defined as inpatient admission≥24 hours in hospital, observation area, emergency department, or other equivalent health care facility, depending on the health care system) or results in death.

Also provided herein are methods of reducing rate of hospitalization due to exacerbations in a human subject with eosinophilic COPD, comprising orally administering to the subject a therapeutically effective amount of dexpramipexole, or a pharmaceutically acceptable salt thereof.

A moderate COPD exacerbation is defined as an acute exacerbation of COPD that requires either systemic corticosteroids (intramuscular (IM), intravenous, or oral) and/or antibiotics.

Also provided herein are methods of reducing the rate of oral/parenteral steroid and/or antibiotic use for treatment of COPD in a human subject with eosinophilic COPD, comprising orally administering to the subject a therapeutically effective amount of dexpramipexole, or a pharmaceutically acceptable salt thereof.

In some embodiments, the subject may further be taking one or more of an oral corticosteroid (OCS), a long-acting muscarinic antagonist (LAMA), a long-acting 32 agonist (LABA), or a bronchodilator.

In some embodiments, the subject may further be taking an oral corticosteroid (OCS) selected from the group consisting of cortisone acetate, dexamethasone, hydrocortisone, methylprednisolone, prednisolone, prednisone, and combinations thereof. Maintenance oral corticosteroids (OCS) are typically prescribed at up to 20 mg of prednisone per day or equivalent. In some embodiments, the subject may further be taking a long-acting muscarinic antagonist (LAMA) selected from the group consisting of umeclidinium, aclidinium, glycopyrronium, glycopyrrolate, tiotropium, and combinations thereof. In some embodiments, the subject may further be taking a long-acting 32 agonist (LABA) selected from the group consisting of formoterol fumarate, salmeterol, salmeterol xinafoate, arformoterol tartrate, olodaterol, vilanterol, indacaterol, and combinations thereof. In some embodiments, the subject may further be taking a medication selected from the group consisting of short-acting bronchodilators, long-acting bronchodilators, ultra-long-acting bronchodilators, xanthines, short-acting muscarinic antagonists, long-acting muscarinic antagonists, leukotriene antagonists, 5-lipoxygenase inhibitors, and combinations thereof.

In some embodiments, the subject may further be taking a biologic. In some embodiments, the biologic targets one or more of the following: OX40, OX40L, IL-25, IL-33, IL-33/ST2, IL-4, IL-4Ra, IL-13, TSLP, or IgE. Examples of biologics include but are not limited to dupilumab, tozorakimab, itepekimab, astegolimab, tezepelumab, amlitelimab, and lunsekimig.

Given the under-reporting and under-recognition of symptoms in COPD, there is a need for appropriate tools in clinical practice to identify symptoms and adjust treatment accordingly. The most efficient and accurate way for physicians to assess symptom severity, activity limitation, and health-related quality of life is to use a standardized measure, such as a validated patient-centered questionnaire. A number of PRO questionnaires and assessments are available for patients with COPD.

The COPD Assessment Test (CAT) aims to measure the impact of COPD on health-related quality of life and to facilitate patient-physician communication. Items covered include physical symptoms such as cough, phlegm, chest tightness, breathlessness when going up hills and stairs, activity limitation at home, and energy.

The modified Medical Research Council (mMRC) dyspnea scale ranges from (0-4) and is easily used to indicate the extent to which dyspnea impacts daily activities.

The Clinical COPD Questionnaire (CCQ) enables a more complete understanding of the impact of COPD on patients, including a more comprehensive assessment of activity limitation and emotional dysfunction. Areas of assessment include symptoms (e.g. dyspnea, cough, and phlegm), functional state, and mental state.

The St. George's Respiratory Questionnaire (SGRQ) includes numerous questions to measure impact on overall health, daily life, and perceived well-being in patients with obstructive airways disease.

The Evaluating Respiratory Symptoms in Chronic Obstructive Pulmonary Disease (E-RS:COPD) is a patient-reported outcome (PRO) measure developed to quantify the severity of respiratory symptoms and test the effects of treatment in clinical trials of stable COPD. The total score represents overall respiratory symptom severity, with 3 sub-scales capturing breathlessness (5 items), cough and sputum (3 items), and chest symptoms (3 items). The 11 items comprising this instrument are part of an existing measure, the 14-item EXAcerbations of COPD Tool (EXACT).

Baseline Dyspnea Index (BDI) is an interviewer-administered rating of severity of dyspnea at a single state. It provides a multidimensional measurement of dyspnea based on 3 components that evoke dyspnea in activities of daily living, in symptomatic individuals. BDI is rated from 0 (very severe) to 4 (no impairment).

Transition Dyspnea Index (TDI) measures changes in dyspnea severity from the baseline as established by the BDI. TDI is rated by seven grades from −3 (major deterioration) to +3 (major improvement).

Clinically important deterioration (CID) is a composite endpoint developed to quantify the impact of pharmacological treatment in clinical trials for COPD, also showing a prognostic value. CID is defined as any of the following condition: FEV₁ decrease≥100 mL from baseline, and/or SGRQ total score increase≥4-unit from baseline, and/or the occurrence of a moderate-to-severe exacerbation of COPD.

The BODE (Body mass index, airflow Obstruction, Dyspnea, and Exercise capacity) index for COPD survival is a multidimensional index of disease severity in COPD that incorporates four independent predictors: the body mass index (BMI), FEV₁, mMRC dyspnea scale, and the exercise capacity assessed by the 6-min walking distance (6MWD) test. Among patients with COPD, the BODE index is a better predictor of disease severity, number of acute exacerbations, and mortality than the FEV₁.

In some embodiments, administering dexpramipexole, or a pharmaceutically acceptable salt thereof, improves a measurement selected from the group consisting of St George's Respiratory Questionnaire for COPD Patients (SGRQ-C), CAT, mMRC, Evaluating Respiratory Symptoms in Chronic Obstructive Pulmonary Disease (E-RS:COPD), COPDCompEx, EXACT, BDI, TDI, CID, BODE index, and any combination thereof.

In some embodiments, administering dexpramipexole, or a pharmaceutically acceptable salt thereof, improves the subject's SGRQ-C total score measurement. In some embodiments the SGRQ-C measurement improves by about 1 to about 40 points. In some embodiments the SGRQ-C measurement improves by about 10 to about 15 points. In some embodiments the SGRQ-C measurement improves by about 10 to about 20 points. In some embodiments the SGRQ-C measurement improves by about 20 to about 30 points. In some embodiments the SGRQ-C measurement improves by about 20 to about 40 points. In some embodiments the SGRQ-C measurement improves by about 5 to about 10 points. In some embodiments the SGRQ-C measurement improves by about 10 to about 15 points. In some embodiments the SGRQ-C measurement improves by about 15 to about 20 points.

In some embodiments, administering dexpramipexole, or a pharmaceutically acceptable salt thereof, improves the subject's CAT total score measurement. In some embodiments the CAT measurement improves by about 1 to about 30 points. In some embodiments the CAT measurement improves by about 10 to about 15 points. In some embodiments the CAT measurement improves by about 10 to about 20 points. In some embodiments the CAT measurement improves by about 20 to about 30 points. In some embodiments the CAT measurement improves by about 5 to about 10 points. In some embodiments the CAT measurement improves by about 10 to about 15 points. In some embodiments the CAT measurement improves by about 15 to about 20 points.

U.S. Food and Drug Administration (FDA) has developed a clinical resource, BEST (Biomarkers, EndpointS, and other Tools), which defines biomarker used in drug development. A Pharmacodynamic/Response Biomarker, herein referred to as “biomarker” is defined as a biomarker that shows that a biological response has occurred in an individual who has been exposed to a medical product, such that one or more expected clinical events linked to that biomarker can be reasonably anticipated. Any reference to symptoms, signs, or biomarkers, are herein collectively referred to as “biomarker.”

Also provided herein are methods of changing the level of one or more biomarkers associated with eosinophilic chronic obstructive pulmonary disorder (COPD), lung function decline, or any combination thereof in a human subject with eosinophilic COPD, comprising orally administering to the subject a therapeutically effective amount of dexpramipexole, or a pharmaceutically acceptable salt thereof.

In some embodiments, the biomarker is selected from the group consisting of blood eosinophil biomarkers, tissue eosinophil biomarkers, imaging biomarkers, inflammatory biomarkers, transcriptomics, proteomics, and any combination thereof.

In some embodiments, the inflammatory biomarkers are selected from the group consisting of type 2 inflammation associated mediators, including IL-1, IL-10, IL-3, IL-4, GM-CSF, IL-5, IL-6, IL-8, IL-13, the TNFα superfamily, IL-33, ST2 (IL1RL1), CCL2, CCL3, CCL4, CCL11, CCL17, CCR3, fibrinogen, eosinophil-derived neurotoxin, eosinophil peroxidase, eosinophil cationic protein, and combinations thereof.

In some embodiments, the tissue eosinophil biomarkers are selected from the following group including nasal eosinophil peroxidase, sputum esoinophil counts, eosinophil counts from bronchial washings/biopsies

In some embodiments, imaging biomarkers are selected from the group of quantitative imaging indices including lung volumes, low attentuation areas (%<−856HU, %<−950JU), DPM air trapping, mucus plug score, airway lumen area, airway wall area, airway wall thickness, airway wall area %, computational airflow analysis, and vascular analysis of blood vessels in the lung.

### EXAMPLES

The following examples are intended solely as an illustration and should not be regarding as restricting the disclosure in any way.

In the examples below, dexpramipexole is orally administered.

### Example 1—Phase II Clinical Trial in Participants with Eosinophilic COPD

Dexpramipexole, the investigational medicinal product, is described in Table 1.

TABLE 1
Investigational Product

| | |
|---|---|
| Product Name: | Dexpramipexole |
| Active Ingredient | Dexpramipexole dihydrochloride |
| Dosage Form: | Film coated tablet |
| Unit Dose | Dexpramipexole dihydrochloride 150 mg (dexpramipexole, |
| | 112 mg base equivalent) administered BID |
| Route of Administration | Oral |
| Physical Description | White, filmcoated, capsule shaped tablets containing the |
| | following excipients: microcrystalline cellulose, mannitol, |
| | crospovidone, and magnesium stearate. The coating |
| | contains polyvinyl alcohol, titanium dioxide, polyethylene |
| | glycol 3350 and talc. |

Abbreviation: BID = twice daily

### Study Rationale

This Phase II pharmacodynamic (PD) study with dexpramipexole in participants with eosinophilic COPD examined the profile and duration of reductions of eosinophils in blood. In addition, baseline-corrected changes in pre- and post-BD FEV₁ and in COPD-validated patient reported outcomes (PROs) were examined at various timepoints. Exploratory endpoints included assessment of mucus plug scores, additional quantitative CT measures and tissue eosinophil biomarkers. This study examined dexpramipexole 150 mg BID, a dose that has a favorable safety profile in participants with ALS and EADs and is under study in the Phase III dexpramipexole asthma program (Studies EXHALE-2, EXHALE-3, and EXHALE-4). Thus, the primary objective of this clinical study was to investigate the pharmacodynamic profile of dexpramipexole in participants with eosinophilic COPD. Additional objectives included examining the safety, tolerability, lung function, PRO, blood and nasal biomarkers and quantitative CT measures in participants with eosinophilic COPD.

### Study Design

This was a Phase II, open-label, multicenter study in approximately 30 participants aged≥40 to ≤80 years of age with a diagnosis of COPD and a blood eosinophil count of ≥0.30×10⁹/L at screening. Some patients with blood eosinophil counts of 0.25-0.299×10⁹/L at screening also were included. Participants were treated with 150 mg dexpramipexole BID. This study helped characterize the pharmacodynamic profile of dexpramipexole in reducing blood eosinophil counts over 16 weeks of treatment, the efficacy of dexpramipexole on pulmonary function and COPD validated patient reported outcomes (PROs), and the safety and tolerability of dexpramipexole in participants with eosinophilic COPD. Informed consent was obtained from participants before the initiation of any study-specific procedures. This study was conducted at approximately 30 sites in the US.

The duration of each participant's involvement in the study was approximately 24 weeks (Screening through Follow-up Phase) or the occurrence of one of the following events, whichever comes first:
A recommendation of discontinuation by the investigator or Sponsor,
A decision by the participant to discontinue for any reason,
A decision by the investigator to discontinue the participant if medically necessary, or
An administrative decision is made to end the study.

There were three phases to the study:
Screening Phase (Screening Visit through Baseline) Participants underwent screening to assess whether they satisfy the eligibility criteria. Participants took part in the screening period (of up to 28 days duration), to confirm that their eosinophil blood count was ≥0.25×10⁹/L and that other screening assessments met eligibility criteria.
Treatment Phase (Baseline through completion of Week 16)
Participant eligibility was assessed during the Screening Visit and confirmed at the Baseline Visit. After the collection of all Baseline assessments, participants began investigational treatment (dexpramipexole 150 mg), BID for 16 weeks. The Week 16 Visit was the Primary Outcome Visit for the study.
Follow-up Phase (Conclusion of Week 16 assessments through Week 20) During this phase, participants had one final phone visit to collect final safety assessments following cessation of investigational product.

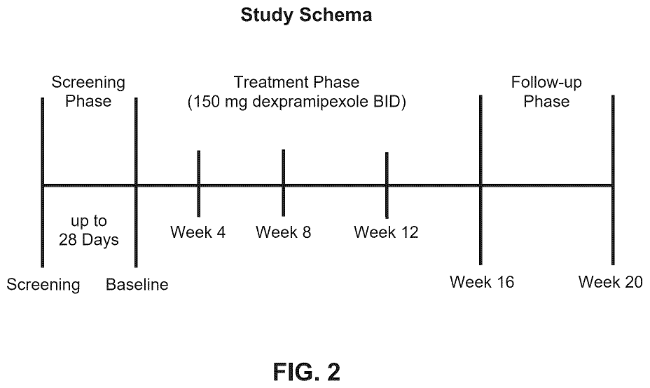

A schematic outline of the clinical study is given in FIG. 2.

### Study Objectives

### Primary Objective

The primary objective of this study was to characterize the pharmacodynamic (PD) profile of dexpramipexole in reducing blood eosinophil counts over 16 weeks of treatment.

### Secondary Objectives

The secondary objectives of this study were:
To assess the efficacy of dexpramipexole on pulmonary function
To assess the efficacy of dexpramipexole on COPD-validated PROs

### Exploratory Objectives

The exploratory objectives of this study were:
To assess the effect of dexpramipexole on inflammatory blood and nasal biomarkers including serum cytokines, proteomics, gene expression and gene signature, and blood and nasal transcriptomics
To characterize the pharmacokinetics (PK) of dexpramipexole in adults with eosinophilic COPD
To assess the effect of dexpramipexole on pulmonary mucus plugs and other quantitative CT measures in the lung in participants with eosinophilic COPD

### Safety Objective

The safety objective of this study was to assess the safety and tolerability of dexpramipexole in participants with eosinophilic COPD.

### Endpoints

### Primary Endpoint

The primary endpoint of this study was geometric mean change in the blood AEC from Baseline to Week 16.

### Secondary Endpoints

The secondary endpoints were:
Pre-BD FEV₁, change from Baseline to Week 16
Post-BD FEV₁, change from Baseline to Week 16
St George's Respiratory Questionnaire for COPD patients (SGRQ-C), change from Baseline to Week 16
COPD Assessment Test (CAT), change from Baseline to Week 16

### Exploratory Endpoints

The exploratory endpoints were:
Change from Baseline on biomarkers including serum inflammatory biomarkers and blood and nasal transcriptomics to Week 16
Change in nasal eosinophil peroxidase concentration from Baseline to Week 16
PK parameters determined from plasma dexpramipexole concentrations and analyzed by population PK modeling with a covariate analysis
Pulmonary mucus plugging score and additional quantitative CT measures, change from Baseline to Week 16

### Safety Endpoints

The safety endpoints of this study were the incidence and severity of treatment emergent AEs, change from Baseline in clinical laboratory parameters, vital signs, and ECG.

### Inclusion Criteria

To be eligible to participate in this study, candidates met the following eligibility criteria at the Screening Visit or at the timepoint specified in the individual eligibility criterion listed below:
1. Signed informed consent form prior to any study-specific procedures.
2. Male or female≥40 to ≤80 years of age at Screening Visit.
3. Physician diagnosis of COPD for at least 24 months prior to screening in accordance with the definition by the American Thoracic Society/European Respiratory Society.
4. Current or former smokers with a cigarette smoking history of ≥10 pack years at Screening calculated as (number of pack years=[number of cigarettes per day/20] multiplied by number of years smoked). Former smokers were defined as those who met the pack-year history but have stopped smoking for at least 6 months prior to Screening.
5. Spirometry: post-bronchodilator FEV₁/FVC<0.70 and post-bronchodilator FEV₁>20% and ≤₆₀% of predicted normal values at Screening calculated using NHANES III reference equations.
6. Documented history of exacerbation risk defined as exacerbation history of ≥1 moderate* or ≥1 severe** within 3 years prior to inclusion.
   Moderate exacerbations were recorded by the investigator and defined as acute exacerbations of COPD that required either systemic corticosteroids (intramuscular, intravenous, or oral) and/or antibiotics.
   Severe exacerbations were recorded by the investigator and defined as acute exacerbations of COPD that required hospitalization. Hospitalization was defined as inpatient admission≥24 hours in hospital, observation area, emergency department, or other equivalent health care facility, depending on the health care system.
7. Background ICS-based therapy (ICS+long-acting β2 agonist [LABA], or ICS+long-acting muscarinic antagonist [LAMA], or ICS+LAMA+LABA) for ≥12 weeks prior to enrollment with a stable dose of medication for ≥4 weeks prior to the Screening Visit.
8. Evidence of an eosinophilic phenotype: Participants with blood eosinophils≥0.30×10⁹/L at the Screening Visit. If the initial value is between 0.250×10⁹/L to 0.299×10⁹/L, then this was repeated once before Baseline/Day 1; the results were obtained prior to Baseline. Some Participants with blood eosinophils≥0.25×10⁹/L at the Screening Visit also were included. If the initial value was between 0.200×10⁹/L to 0.249×10⁹/L, then the blood eosinophil measurement was repeated once before Baseline/Day 1; the results were obtained prior to Baseline.
9. Negative urine pregnancy test for women of childbearing potential (WOCBP) at the Screening and Baseline Visits.
10. WOCBP used either of the following methods of birth control, from Screening through the End of Study Visit:
   a. A highly effective form of birth control (confirmed by the investigator). Highly effective forms of birth control include: true sexual abstinence, a vasectomized sexual partner, Implanon, female sterilization by tubal occlusion, any effective intrauterine device (IUD), IUD/intrauterine system (IUS), Levonorgestrel Intrauterine system, or oral contraceptive; or
   b. Two protocol acceptable methods of contraception in tandem.
   c. Women not of childbearing potential were defined as women who were either permanently sterilized (hysterectomy, bilateral oophorectomy, or bilateral salpingectomy), or who are postmenopausal. Women were considered postmenopausal if they had been amenorrheic for at least 12 months prior to the planned date of the Baseline Visit without an alternative medical cause. The following age specific requirements applied:
   d. Women≤50 years old were considered postmenopausal if they had been amenorrheic for 12 months or more following cessation of exogenous hormonal treatment and follicle stimulating hormone levels in the postmenopausal range.
   e. Women≥50 years old were considered postmenopausal if they had been amenorrheic for 12 months or more following cessation of all exogenous hormonal treatment.
11. Participants must have had the ability to read and write.

### Exclusion Criteria

Study participant candidates were excluded from study entry if any of the following exclusion criteria existed at the Screening Visit or at the timepoint specified in the individual criterion listed below:
1. A current diagnosis of asthma or any history of asthma diagnosis when ≥40 years of age.
2. Significant pulmonary disease other than COPD (eg, α-1 antitrypsin deficiency, active tuberculosis, lung fibrosis, sarcoidosis, interstitial lung disease, pulmonary hypertension, lung cancer, clinically significant bronchiectasis, Churg-Strauss Syndrome) or another diagnosed pulmonary or systemic disease associated with elevated peripheral eosinophil counts.
3. Routine treatment with supplemental oxygen more than 4.0 L/min.
4. Hypercapnia requiring bilevel positive airway pressure (BiPAP).
5. Pneumonia, upper or lower respiratory tract infection or acute exacerbation of COPD within 4 weeks prior to or during the screening period.
6. History of, or planned pneumonectomy, lung transplant, lobectomy, or lung volume reduction surgery.
7. Participants who were participating in the acute phase of a pulmonary rehabilitation program, ie, who started rehabilitation.
8. Treatment with a biologic investigational drug in the last 5 months prior to Screening. Treatment with non-biologic investigational drugs in the previous 30 days or five-half-lives prior to Screening, whichever was longer. Treatment with GSK3511294 (long-acting anti-interleukin-5) in the past 12 months.
9. Treatment with any of the following monoclonal antibody therapies within 120 days prior to Baseline: benralizumab, dupilumab, mepolizumab, reslizumab, omalizumab, tezepelumab, or tralokinumab.
10. Treatment with pramipexole (Mirapex®) within 30 days of Baseline.
11. Treatment with selected drugs known to have a substantial risk of neutropenia in the past 30 days prior to Screening.
12. Treatment with selected drugs known to have a substantial risk of QT prolongation in the past 30 days prior to the Screening Visit.
13. Known or suspected alcohol or drug abuse.
14. Uncontrolled severe hypertension: systolic blood pressure>180 mmHg or diastolic blood pressure>110 mmHg prior to the Baseline Visit despite anti-hypertensive therapy.
15. History of malignancy that required surgery (excluding local and wide-local excision), radiation therapy and/or systemic therapy during the 2 years prior to the Baseline Visit.
16. History of human immunodeficiency virus (HIV) infection or chronic infection with hepatitis B or C.
17. A helminth parasitic infection diagnosed within 24 weeks prior to Screening that had not been treated with or had failed to respond to standard of care therapy.
18. Medical or other condition likely to interfere with participant's ability to undergo study procedures, adhere to visit schedule, or comply with study requirements.
19. Known or suspected noncompliance with medication.
20. Unwillingness or inability to follow the procedures outlined in the protocol.
21. Neutrophil count<2.000×10⁹/L at Screening
22. Renal dysfunction, defined as an estimated glomerular filtration rate (eGFR)<60 mL/min/1.73 m² at Screening (using the Chronic Kidney Disease Epidemiology Collaboration) formula
23. Active liver disease defined as any known current infectious, neoplastic, or metabolic pathology of the liver or unexplained elevations in alanine aminotransferase (ALT), aspartate aminotransferase (AST), >3× the upper limit of normal (ULN), or total bilirubin>2×ULN at Screening confirmed by a repeat abnormal measurement of the relevant value(s), at least 1 week apart.
24. History of New York Heart Association class IV heart failure or last known left ventricular ejection fraction<25%.
25. History of major adverse cardiovascular event (MACE) within 3 months prior to the Baseline Visit.
26. History of cardiac arrhythmia within 3 months prior to the Baseline Visit that was not controlled by medication or via ablation.
27. History of long QT syndrome.
28. Corrected QT interval by Fridericia (QTcF) interval>450 ms for males and >470 ms for females at Screening or QTcF≥480 ms for participants with bundle branch block.
29. Clinically important abnormalities in resting ECG that may interfere with the interpretation of QTcF interval changes at Screening, including resting heart rate<45 beats per minute (bpm) or >100 bpm.
30. Metallic implants or artefacts in the chest, including pacemakers, surgical clips, implantable cardioverter defibrillators, wire sutures or rods that significantly interfere with CT scan readings (only for participants that had CT imaging).
31. Pregnant women or women breastfeeding.
32. Males who were unwilling to use an acceptable method of birth control during the entire study period (ie, condom with spermicide).
33. Employees of the clinical study center or family members (first-degree relatives) of such individuals or anyone involved in the planning and/or conduct of the study.

### Study Participants

Table 2 summarizes the demographics of all the patients that enrolled in the trial.

TABLE 2
Demographics

| | |
|---|---|
| | Dexpramipexole |
| | 150 mg BID |
| Category | (N = 7) |

Age (yrs) [n]

| | |
|---|---|
| Mean ± SD | 64.4 ± 9.22 |
| Median | 65.0 |
| Q1, Q3 | 56, 71 |
| Min, Max | 53, 80 |

Age Classification [n (%)]

| | | |
|---|---|---|
| 40 to <65 years | 3 | (42.9) |
| >=65 years | 4 | (57.1) |

Gender [n (%)]

| | | |
|---|---|---|
| Male | 4 | (57.1) |
| Female | 3 | (42.9) |

Is this female a woman of child-bearing potential* [n (%)]

| | | |
|---|---|---|
| Yes | 0 | (0.0) |
| No | 3 | (100.0) |

Race [n (%)]

| | | |
|---|---|---|
| American Indian or Alaska Native | 0 | (0.0) |
| Asian | 0 | (0.0) |
| Black or African American | 2 | (28.6) |
| Native Hawaiian or Other Pacific Islander | 0 | (0.0) |
| White | 5 | (71.4) |
| Other | 0 | (0.0) |

Ethnicity [n (%)]

| | | |
|---|---|---|
| Hispanic or Latino | 1 | (14.3) |
| Not Hispanic or Latino | 6 | (85.7) |
| Not Reported | 0 | (0.0) |

Height (cm) [n]

| | |
|---|---|
| Mean ± SD | 168.9 ± 6.28 |
| Median | 167.0 |
| Q1, Q3 | 165, 175 |
| Min, Max | 159, 175 |

Weight at Screening (kg) [n]

| | |
|---|---|
| Mean ± SD | 77.4 ± 16.50 |
| Median | 76.0 |
| Q1, Q3 | 61, 95 |
| Min, Max | 55, 97 |

BMI at Screening (kg/m\hat{ }2) [n]

| | |
|---|---|
| Mean ± SD | 27.1 ± 5.13 |
| Median | 27.3 |
| Q1, Q3 | 23, 31 |
| Min, Max | 20, 35 |

Weight at Baseline (kg) [n]

| | |
|---|---|
| Mean ± SD | 77.4 ± 16.50 |
| Median | 76.0 |
| Q1, Q3 | 61, 95 |
| Min, Max | 55, 97 |

BMI at Baseline (kg/m\hat{ }2) [n]

| | |
|---|---|
| Mean ± SD | 27.1 ± 5.13 |
| Median | 27.3 |
| Q1, Q3 | 23, 31 |
| Min, Max | 20, 35 |

*Percentages are based on number of females.

### Schedule of Events/Assessments

The Schedule of Assessments (SOA) is provided in Table 3.

TABLE 3
Schedule of Assessments

| | | | | | | | Safety FU | | |
|---|---|---|---|---|---|---|---|---|---|
| | | | Treatment | | | | Week 20² | IP | |
| | Screening | | Week | Week | Week | Week | Phone | Discontinuation | Early |
| | Screen | Baseline | 4 | 8 | 12 | 16 | FU | IP | Terminatio |
| | Screen | Baseline/ | V2 | V3 | V4 | V5 | 4 weeks after | Discon³ | ET³ |
| | Up to | Day 1¹ | ±3¹⁴ | ±3¹⁴ | ±3¹⁴ | ±3¹⁴ | last dose ± | N/A | N/A |
| Event | Day −28 | V1 | days | days | days | days | 3¹⁴ days | N/A | N/A |
| Signed/confirm ICF | X | X | | | | | | | |
| Assessment of Eligibility | X | X | | | | | | | |
| Medical Hx/Demographics | X | | | | | | | | |
| Smoking Status | X | | | | | X | | X | X |
| Smoking Cessation | X | | | | | X | | X | X |
| Counseling | | | | | | | | | |
| SGRQ-C | | X | | | | X | | X | X |
| COPD Assessment Test | | X | | | | X | | X | X |
| Physical Exam (w/body | X | X | | | | X | | X | X |
| weight and height at | | | | | | | | | |
| Screening only) | | | | | | | | | |
| Vital signs⁴ | X | X | X | X | X | X | | X | X |
| 12-lead ECG⁵ | X | X⁶ | X⁷ | | | X⁷ | | X⁷ | X⁷ |
| CT Scan | | X¹³ | | | | X¹³ | | X | X |
| CBC with differential | X⁸ | X | X | X | X | X | | X | X |
| Chemistry | X | X | X | | | X | | X | X |
| Urinalysis | X | X | | | | X | | X | X |
| FSH | X⁹ | | | | | | | | |
| Urine Pregnancy Test | X | X | X | X | X | X | | X | X |
| Contraception counseling | X | X | X | X | X | X | | X | X |
| Adverse events | | X | X | X | X | X | X | X | X |
| Prior and Concomitant | X | X | X | X | X | X | X | X | X |
| medications | | | | | | | | | |
| Exacerbation assessment | X | X | X | X | X | X | X | X | X |
| Pre-BD spirometry¹⁰ | X | X | X | X | X | X | | X | X |
| Post-BD spirometry¹⁰ | X | X | | | | X | | X | X |
| Investigational Product | | X | X | X | X | | | | |
| Dispensing | | | | | | | | | |
| In Clinic Dosing of IP | | X | X | X | X | | | | |
| Investigational product | | | X | X | X | X | | X | X |
| Accountability | | | | | | | | | |
| Plasma for dexpramipexole | | X | X | X | X | X | | | |
| concentration¹¹ | | | | | | | | | |
| Plasma for biomarkers | | X | | | | X | | | |
| Blood and nasal brushing | | X | | | | X | | | |
| for transcriptomics¹⁵ | | | | | | | | | |
| Nasal swab for eosinophil | | X | | | | X | | | |
| peroxidase | | | | | | | | | |

Abbreviations: CBC = complete blood count;
COPD = chronic obstructive pulmonary disease;
ECG = electrocardiogram;
ET = Early Termination;
FEV₁ = forced expiratory volume in 1 second;
FSH = follicle stimulating hormone;
FU = follow = up;
Hx-history;
ICF = informed consent form;
ICS = inhaled corticosteroid;
IP = investigational product;
LABA = long-acting β2 agonist;
N/A = not applicable;
PK = pharmacokinetic;
post-BD = post-bronchodilator;
pre-BD = pre-bronchodilator;
SGRQ-C = St George's Respiratory Questionnaire for COPD Patients

1. Baseline Visit (Day 1) was performed after results had been received from screening testing to determine eligibility.
2. The Safety Follow-up Phone Visit was performed within 4 weeks±3 days after stopping investigational product. An amendment to this protocol allowed patients to have the Safety Follow-up Phone Visit+/−7 days from 4 weeks.
3. For participants that prematurely discontinue IP but are remaining in the study, the IP Discontinuation Visit should be performed within 7 days of the last dose of IP. For participants withdrawn from the study prematurely, the Early Termination Visit was performed as soon as possible following discontinuation. An amendment to this protocol excluded patients that prematurely discontinued IP from remaining in the study.
4. Vital signs were measured in the seated upright position after the participant had been rested for 5 minutes (temperature, systolic and diastolic systemic blood pressure in mmHg, respiratory rate per minute, and pulse. per minute).
5. All 12-lead ECGs were collected in the supine position after the participant had been rested for 5 minutes. Screening ECG was a single tracing and was done before spirometry, blood draws, and nasal swabs and brushings.
6. Baseline 12-lead ECG was collected as triplicate tracings, with each collected at least 1 minute apart.
7. Post-baseline 12-lead ECGs were a single tracing and locally evaluated with the option to repeat if concerning findings are seen. In case of a repeat, interval durations should be calculated as the mean across repeats. Alternatively, in the case of a repeat, the interval duration was calculated as the final final measurement.
8. The qualifying eosinophil count was drawn at the Screening Visit. If the eosinophil count at Screening was uninterpretable or between 0.250×109/L to 0.299×109/L, then it was repeated once at an unscheduled visit (prior to Baseline/Day 1). Results were available prior to Baseline. An amendment to this protocol allowed patients with an eosinophil count between 0.200×109/L to 0.249×109/L to have a repeated measurement once at an unscheduled visit (prior to Baseline/Day 1). Serum FSH levels were tested at screening in women<50 years old who are considered postmenopausal, see Inclusion Criterion 9c.
9. Spirometry (FEV1) was an efficacy endpoint and was collected between 6 AM and 12 noon, to minimize diurnal variation. Ideally, each session was at approximately the same time of day throughout the study. Post-BD spirometry testing was within 15 to 30 minutes after inhalation of albuterol/salbutamol.
10. Blood samples for the determination of plasma dexpramipexole concentration were collected at predose.
11. When an ECG was collected at the same timepoint, the ECG was conducted first, followed immediately by the transcriptomics sampling.
12. At sites selected for CT imaging, CT scans were completed within 7 days prior to the visit or during the visit. Eligibility from the Screening Visit was confirmed prior to the Baseline Visit CT scan.
13. An amendment to this protocol allowed patients to have measurements taken+/−7 days from the indicated week.
14. An amendment to this protocol directed nasal samples to be taken via nasal brushing, rather than nasal swab.

### Assessment of Efficacy

The following clinical tests/assessments were performed to assess the efficacy of dexpramipexole, as found in the subsections below.

### Primary Endpoint—Peripheral Blood Absolute Eosinophil Count

Blood eosinophil counts were obtained at every onsite study visit. Samples were collected between 6 AM and 12 noon to minimize diurnal variation. Table 4 provides a summary of AEC for participants that completed 16 weeks of treatment. On average, AEC decreased to 21.3% of the baseline value by week 16.

TABLE 4
Summary of Absolute Eosinophil Count (AEC) (cells/uL) by Visit

| | | Dexpramipexole 150 | |
|---|---|---|---|
| | | mg BID (N = 7) | |
| | | | Ratio to |
| Visit | Statistic | Observed | baseline |
| Baseline | n | 7 | |
| | Geometric Mean ± GSE* | 387.9 ± 1.11 | |
| | Median | 414.5 | |
| | Q1, Q3 | 310, 480 | |
| | Min, Max | 250, 551 | |
| Week 4 | n | 7 | 7 |
| | Geometric Mean ± GSE* | 353.3 ± 1.22 | 0.911 ± 1.1189 |
| | Median | 298.0 | 0.799 |
| | Q1, Q3 | 218, 676 | 0.70, 1.23 |
| | Min, Max | 192, 724 | 0.70, 1.51 |
| Week 8 | n | 6 | 6 |
| | Geometric Mean ± GSE* | 118.0 ± 1.95 | 0.323 ± 1.9860 |
| | Median | 212.4 | 0.658 |
| | Q1, Q3 | 90, 336 | 0.19, 0.96 |
| | Min, Max | 5, 396 | 0.01, 1.08 |
| Week 12 | n | 7 | 7 |
| | Geometric Mean ± GSE* | 64.9 ± 2.30 | 0.167 ± 2.2497 |
| | Median | 224.0 | 0.538 |
| | Q1, Q3 | 6, 589 | 0.01, 1.07 |
| | Min, Max | 5, 589 | 0.01, 1.90 |
| Week 16 | n | 7 | 7 |
| | Geometric Mean ± GSE* | 82.8 ± 2.06 | 0.213 ± 2.0849 |
| | Median | 260.0 | 0.620 |
| | Q1, Q3 | 6, 342 | 0.01, 0.64 |
| | Min, Max | 5, 384 | 0.01, 1.24 |

*GSE = Standard Error of the Geometric Mean

The AEC measurements for each individual who completed week 16 are provided in Table 5. Of the 7 subjects that completed week 16, 3 were classified as AECR≥0.5, indicated by having a reduction in AEC of ≥50% from baseline at week 16. Subjects classified as AECR<0.5 are defined as subjects who had a reduction in AEC of <50% from baseline at week 16.

TABLE 5
Absolute Eosinophil Count (AEC) (cells/uL)

| | AEC week 16 | | | Study | Result | Ratio to |
|---|---|---|---|---|---|---|
| Subject ID | reduction category | Visit* | Date/Time | Day | (cells/uL) | Baseline |
| 10001-001-0001 | <50% AEC | Screening | 2024 Jul. 9/10:17 | −27 | 248 | |
| | reduction at Week 16 | Screening | 2024 Jul. 23/08:06 | −13 | 453 | |
| | | (Screening Retest) | | | | |
| | | Day 1 | 2024 Aug. 6/09:35 | 1 | 383 | |
| | | Baseline** | | | 417 | |
| | | Week 4 | 2024 Aug. 28/09:55 | 23 | 290 | 0.70 |
| | | Week 8 | 2024 Oct. 1/09:35 | 57 | 235 | 0.56 |
| | | Week 12 | 2024 Oct. 30/09:30 | 86 | 224 | 0.54 |
| | | Week 16 | 2024 Nov. 26/10:20 | 113 | 260 | 0.62 |
| 10001-001-0002 | >=50% AEC | Screening | 2024 Jul. 22/11:10 | −13 | 423 | |
| | reduction at Week 16 | Day 1 | 2024 Aug. 5/09:50 | 1 | 329 | |
| | | Baseline** | | | 373 | |
| | | Week 4 | 2024 Aug. 29/10:10 | 25 | 298 | 0.80 |
| | | Week 8 | 2024 Oct. 1/10:00 | 58 | 0 | 0.01(a) |
| | | Week 12 | 2024 Oct. 30/10:02 | 87 | 0 | 0.01(a) |
| | | Week 16 | 2024 Dec. 2/11:40 | 120 | 0 | 0.01(a) |
| 10001-008-0002 | <50% AEC reduction | Screening | 2024 Sep. 9/12:10 | −24 | 452 | |
| | at Week 16 | Day 1 | 2024 Oct. 4/12:58 | 1 | 213 | |
| | | Baseline** | | | 310 | |
| | | Week 4 | 2024 Nov. 8/12:18 | 36 | 218 | 0.70 |
| | | Week 8 | 2024 Dec. 5/11:30 | 63 | 336 | 1.08 |
| | | Week 12 | 2025 Jan. 3/11:40 | 92 | 589 | 1.90 |
| | | Week 16 | 2025 Jan. 30/11:48 | 119 | 384 | 1.24 |
| 10001-010-0005 | <50% AEC reduction | Screening | 2024 Nov. 26/09:13 | −14 | 419 | |
| | at Week 16 | Day 1 | 2024 Dec. 11/08:05 | 1 | 410 | |
| | | Baseline** | | | 414 | |
| | | Week 4 | 2025 Jan. 8/08:22 | 29 | 388 | 0.94 |
| | | Week 8 | 2025 Feb. 5/08:01 | 57 | 396 | 0.96 |
| | | Week 12 | 2025 Mar. 4/07:52 | 84 | 231 | 0.56 |
| | | Week 16 | 2025 Mar. 26/09:08 | 106 | 266 | 0.64 |
| 10001-022-0001 | >=50% AEC reduction | Screening | 2024 Oct. 14/10:27 | −27 | 412 | |
| | at Week 16 | Day 1 | 2024 Nov. 11/10:26 | 1 | 559 | |
| | | Baseline** | | | 480 | |
| | | Week 4 | 2024 Dec. 10/09:36 | 30 | 724 | 1.51 |
| | | Week 8 | 2025 Jan. 7/08:59 | 58 | 90 | 0.19 |
| | | Week 12 | 2025 Feb. 3/09:10 | 85 | 6 | 0.01 |
| | | Week 16 | 2025 Mar. 7/10:07 | 117 | 6 | 0.01 |
| 10001-022-0006 | <50% AEC reduction | Screening | 2024 Oct. 22/13:01 | −22 | 567 | |
| | at Week 16 | Day 1 | 2024 Nov. 14/11:32 | 1 | 536 | |
| | | Baseline** | | | 551 | |
| | | Week 4 | 2024 Dec. 12/11:18 | 29 | 676 | 1.23 |
| | | Week 12 | 2025 Jan. 31/10:45 | 79 | 589 | 1.07 |
| | | Week 16 | 2025 Mar. 12/09:34 | 119 | 342 | 0.62 |
| 10001-024-0002 | >=50% AEC reduction | Screening | 2024 Nov. 22/10:21 | −18 | 208 | |
| | at Week 16 | Screening Retest | 2024 Dec. 2/08:32 | −8 | 273 | |
| | | Day 1 | 2024 Dec. 11/09:20 | 1 | 229 | |
| | | Baseline** | | | 250 | |
| | | Week 4 | 2025 Jan. 8/09:57 | 29 | 192 | 0.77 |
| | | Week 8 | 2025 Feb. 7/09:27 | 59 | 192 | 0.77 |
| | | Week 12 | 2025 Mar. 5/09:30 | 85 | 9 | 0.04 |
| | | Week 16 | 2025 Mar. 26/09:56 | 106 | 98 | 0.39 |

*Unscheduled (e.g., Early Term) shown in parentheses is windowed per SAP to the nominal visit presented.
**Baseline is derived as the geometric mean of the screening and Day 1 (e.g., Screening or Screening Retest, and Day 1) results prior to first dose of study treatment.
(a)Zero values are replaced with 5 cells/>L, which is 50% of the lower limit of quantification.

### Spirometry

### Pre-BD Spirometry

For all in clinic site visits, participants were instructed to withhold their COPD medication on the morning of the visit, per Table 6.
Pre-bronchodilator spirometry was collected between 6 AM and 12 noon local time and was collected within ±2 hours of the Baseline Visit to minimize diurnal variation.
Every effort was made to collect the Week 16 Pre-BD spirometry within +1 hour of the time of day that the Baseline spirometry was collected.

Participants who presented for Screening but had not withheld their COPD medication (Table 6) had all other Screening assessments performed and were requested to reschedule the Screening spirometry as soon as feasible within the visit window. Participants who presented for subsequent visits through Week 16 but had not withheld their COPD medication or investigational product were requested to reschedule the visit as soon as feasible within the visit window.

Spirometry was collected according to the American Thoracic Society/European Respiratory Society guidelines on standardization of spirometry according to the Schedule of Assessments (Table 3). Spirometry was obtained on all participants during the study.

TABLE 6
Intervals for Holding COPD Medications Prior to Spirometry

| | | Minimum washout |
|---|---|---|
| | Examples | prior to |
| | (proprietary names | performing |
| Medication | Capitalized) | Spirometry |
| Short-acting | albuterol, salbutamol, | 6 hours |
| bronchodilator | levalbuterol, Xopenex | |
| Long-acting | Advair, arformoterol, Dulera, | 12 hours |
| bronchodilator | formoterol, olodaterol, | |
| | Symbicort, salmeterol | |
| Ultra-long-acting | Breo, vilanterol, Arcapta, | 24 hours |
| bronchodilator | indacaterol | |
| Xanthines | theophylline | 12 hours |
| Short-acting | Atrovent, ipratropium | 8 hours |
| muscarinic | | |
| antagonist | | |
| Long-acting | aclidinium, glycopyrrolate, | 24 hours |
| muscarinic | glycopyrronium, tiotropium, | |
| antagonist | umeclidinium | |
| Leukotriene | Accolate, montelukast, | 12 hours |
| antagonist | Singulair, zafirlukast | |
| 5-Lipoxygenase | zileuton, Zyflo | 6 hours (immediate |
| Inhibitor | | release) |
| | | 12 hours (extended |
| | | release) |

A summary of FEV₁ 1 values measured prior to bronchodilation, separated by AECR≥0.5 and AECR<0.5 is provided in Table 7. AECR≥0.5 patients displayed an improvement in FEV₁ 1, with the average increase in FEV₁ 1 for AECR≥0.5 patients ranging from 18.7% to 25.3%, corresponding to absolute increases of 220 mL to 263.3 mL.

TABLE 7
Pre bronchodilator: Forced Expiratory Volume in 1 Second (FEV1) [mL]

| | | Dexpramipexole 150 mg BID | | | | | |
|---|---|---|---|---|---|---|---|
| | | (N = 7) | | | | | |
| | | >=50% AEC reduction at Week 16 | | | <50% AEC reduction at Week 16 | | |
| | | (N = 3) | | | (N = 4) | | |
| Visit | Statistic | Observed | CFB* | % CFB* | Observed | CFB* | % CFB* |
| Screening | n | 3 | | | 4 | | |
| | Mean ± SD | 1260.0 ± 599.08 | | | 1702.5 ± 1060.33 | | |
| | Median | 1380.0 | | | 1570.0 | | |
| | Q1, Q3 | 610, 1790 | | | 1055, 2350 | | |
| | Min, Max | 610, 1790 | | | 550, 3120 | | |
| Day 1 | n | 3 | | | 4 | | |
| | Mean ± SD | 1233.3 ± 483.46 | | | 1472.5 ± 520.02 | | |
| | Median | 1300.0 | | | 1490.0 | | |
| | Q1, Q3 | 720, 1680 | | | 1100, 1845 | | |
| | Min, Max | 720, 1680 | | | 830, 2080 | | |
| Baseline | n | 3 | | | 4 | | |
| | Mean ± SD | 1233.3 ± 483.46 | | | 1472.5 ± 520.02 | | |
| | Median | 1300.0 | | | 1490.0 | | |
| | Q1, Q3 | 720, 1680 | | | 1100, 1845 | | |
| | Min, Max | 720, 1680 | | | 830, 2080 | | |
| Week 4 | n | 3 | 3 | 3 | 4 | 4 | 4 |
| | Mean ± SD | 1540.0 ± 678.16 | 306.7 ± 436.16 | 24.8 ± 32.69 | 1260.0 ± 511.53 | −212.5 ± 173.47 | −16.4 ± 15.81 |
| | Median | 1720.0 | 70.0 | 9.7 | 1420.0 | −215.0 | −13.3 |
| | Q1, Q3 | 790, 2110 | 40, 810 | 2, 62 | 935, 1585 | −355, −70 | −28, −4 |
| | Min, Max | 790, 2110 | 40, 810 | 2, 62 | 520, 1680 | −400, −20 | −37, −1 |
| Week 8 | | 3 | 3 | 3 | 4 | 4 | 4 |
| | Mean ± SD | 1453.3 ± 616.47 | 220.0 ± 413.88 | 18.7 ± 30.56 | 1755.0 ± 860.79 | 282.5 ± 410.23 | 16.0 ± 19.59 |
| | Median | 1590.0 | 60.0 | 8.3 | 1600.0 | 165.0 | 13.2 |
| | Q1, Q3 | 780, 1990 | −90, 690 | −5, 53 | 1215, 2295 | −5, 570 | 1, 31 |
| | Min, Max | 780, 1990 | −90, 690 | −5, 53 | 880, 2940 | −60, 860 | −4, 41 |
| Week 12 | n | 3 | 3 | 3 | 4 | 4 | 4 |
| | Mean ± SD | 1496.7 ± 448.14 | 263.3 ± 215.02 | 25.3 ± 19.37 | 1672.5 ± 886.84 | 200.0 ± 449.52 | 10.1 ± 21.78 |
| | Median | 1730.0 | 260.0 | 36. | 1440.0 | 45.0 | 4.2 |
| | Q1, Q3 | 980, 1780 | 50, 480 | 3, 37 | 1145, 2200 | −55, 455 | −3, 23 |
| | Min, Max | 980, 1780 | 50, 480 | 3, 37 | 870, 2940 | −150, 860 | −9, 41 |
| Week 16 | n | 3 | 3 | 3 | 4 | 4 | 4 |
| | Mean ± SD | 1523.3 ± 657.29 | 290.0 ± 340.73 | 22.5 ± 25.80 | 1280.0 ± 399.67 | −192.5 ± 318.58 | −11.4 ± 15.14 |
| | Median | 1820.0 | 140.0 | 8.3 | 1370.0 | −80.0 | −8.5 |
| | Q1, Q3 | 770, 1980 | 50, 680 | 7, 52 | 1020, 1540 | −385, 0 | −22, 0 |
| | Min, Max | 770, 1980 | 50, 680 | 7, 52 | 720, 1660 | −660, 50 | −32, 3 |

*CFB = Change from baseline, % CFB = Percent change from baseline.

Measurements of pre-bronchodilation Forced Vital Capacity (FVC), Forced Expiratory Flow 25-75% (FEF₂₅₋₇₅), and Peak Flow are provided in Tables 8, 9, and 10 respectively. These data are separated by AECR≥0.5 and AECR<0.5. Among AECR≥0.5 patients, the average increase in FVC ranged from 8.8% to 15.7%, which corresponded to absolute increases of 216.7 ml to 380 mL (Table 8). The average FEF₂₅₋₇₅ values for AECR≥0.5 patients increased by 6.3% to 13.6%, which corresponds to absolute increases of 20 mL and 116.7 mL. (Table 9). The average improvement in Peak Flow for AECR≥0.5 patients ranged from 25.2% to 63.4% (Table 10).

TABLE 8
Pre bronchodilator Forced Vital Capacity (FVC) [mL]

| | | Dexpramipexole 150 mg BID | | | | | |
|---|---|---|---|---|---|---|---|
| | | (N = 7) | | | | | |
| | | >=50% AEC reduction at Week 16 | | | <50% AEC reduction at Week 16 | | |
| | | (N = 3) | | | (N = 4) | | |
| Visit | Statistic | Observed | CFB* | % CFB* | Observed | CFB* | % CFB* |
| Screening | n | 3 | | | 4 | | |
| | Mean ± SD | 2406.7 ± 566.16 | | | 3080.0 ± 884.72 | | |
| | Median | 2100.0 | | | 3155.0 | | |
| | Q1, Q3 | 2060, 3060 | | | 2400, 3760 | | |
| | Min, Max | 2060, 3060 | | | 1980, 4030 | | |
| Day 1 | n | 3 | | | 4 | | |
| | Mean ± SD | 2410.0 ± 262.87 | | | 2995.0 ± 482.11 | | |
| | Median | 2520.0 | | | 2905.0 | | |
| | Q1, Q3 | 2110, 2600 | | | 2620, 3370 | | |
| | Min, Max | 2110, 2600 | | | 2550, 3620 | | |
| Baseline | n | 3 | | | 4 | | |
| | Mean ± SD | 2410.0 ± 262.87 | | | 2995.0 ± 482.11 | | |
| | Median | 2520.0 | | | 2905.0 | | |
| | Q1, Q3 | 2110, 2600 | | | 2620, 3370 | | |
| | Min, Max | 2110, 2600 | | | 2550, 3620 | | |
| Week 4 | n | 3 | 3 | 3 | 4 | 4 | 4 |
| | Mean ± SD | 2790.0 ± 497.59 | 380.0 ± 379.87 | 15.7 ± 14.81 | 2755.0 ± 729.41 | −240.0 ± 350.90 | −9.0 ± 13.97 |
| | Median | 2690.0 | 240.0 | 11.4 | 2875.0 | −95.0 | −2.9 |
| | Q1, Q3 | 2350, 3330 | 90, 810 | 3, 32 | 2215, 3295 | −450, −30 | −17, −1 |
| | Min, Max | 2350, 3330 | 90, 810 | 3, 32 | 1790, 3480 | −760, −10 | −30, 0 |
| Week 8 | n | 3 | 3 | 3 | 4 | 4 | 4 |
| | Mean ± SD | 2626.7 ± 477.53 | 216.7 ± 344.29 | 8.8 ± 13.54 | 3162.5 ± 792.10 | 167.5 ± 353.02 | 4.5 ± 11.70 |
| | Median | 2570.0 | 70.0 | 3.3 | 3175.0 | 185.0 | 5.1 |
| | Q1, Q3 | 2180, 3130 | −30, 610 | −1, 24 | 2500, 3825 | −120, 455 | −5, 14 |
| | Min, Max | 2180, 3130 | −30, 610 | −1, 24 | 2310, 3990 | −240, 540 | −9, 17 |
| Week 12 | n | 3 | 3 | 3 | 4 | 4 | 4 |
| | Mean ± SD | 2690.0 ± 181.93 | 280.0 ± 90.00 | 12.0 ± 5.17 | 2942.5 ± 825.44 | −52.5 ± 345.68 | −3.1 ± 11.75 |
| | Median | 2790.0 | 280.0 | 11.1 | 2830.0 | −75.0 | −2.9 |
| | Q1, Q3 | 2480, 2800 | 190, 370 | 7, 18 | 2300, 3585 | −320, 215 | −12, 6 |
| | Min, Max | 2480, 2800 | 190, 370 | 7, 18 | 2120, 3990 | −430, 370 | −17, 10 |
| Week 16 | n | 3 | 3 | 3 | 4 | 4 | 4 |
| | Mean ± SD | 2743.3 ± 470.57 | 333.3 ± 351.62 | 13.7 ± 13.73 | 2900.0 ± 503.26 | −95.0 ± 203.55 | −3.2 ± 7.22 |
| | Median | 2660.0 | 210.0 | 10.0 | 2985.0 | −80.0 | −2.0 |
| | Q1, Q3 | 2320, 3250 | 60, 730 | 2, 29 | 2510, 3290 | −270, 80 | −9, 3 |
| | Min, Max | 2320, 3250 | 60, 730 | 2, 29 | 2250, 3380 | −300, 80 | −12, 3 |

*CFB = Change from baseline, % CFB = Percent change from baseline.

TABLE 9
Pre bronchodilator Forced Expiratory Flow 25-75% (FEF₂₅₋₇₅) [mL/s]

| | | Dexpramipexole 150 mg BID | | | | | |
|---|---|---|---|---|---|---|---|
| | | (N = 7) | | | | | |
| | | >=50% AEC reduction at Week 16 | | | <50% AEC reduction at Week 16 | | |
| | | (N = 3) | | | (N = 4) | | |
| Visit | Statistic | Observed | CFB* | % CFB* | Observed | CFB* | % CFB* |
| Screening | n | 3 | | | 4 | | |
| | Mean ± SD | 616.7 ± 370.72 | | | 1042.5 ± 1316.93 | | |
| | Median | 800.0 | | | 505.0 | | |
| | Q1, Q3 | 190, 860 | | | 290, 1795 | | |
| | Min, Max | 190, 860 | | | 160, 3000 | | |
| Day 1 | n | 3 | | | 4 | | |
| | Mean ± SD | 620.0 ± 367.56 | | | 675.0 ± 521.44 | | |
| | Median | 730.0 | | | 535.0 | | |
| | Q1, Q3 | 210, 920 | | | 290, 1060 | | |
| | Min, Max | 210, 920 | | | 240, 1390 | | |
| Baseline | n | 3 | | | 4 | | |
| | Mean ± SD | 620.0 ± 367.56 | | | 675.0 ± 521.44 | | |
| | Median | 730.0 | | | 535.0 | | |
| | Q1, Q3 | 210, 920 | | | 290, 1060 | | |
| | Min, Max | 210, 920 | | | 240, 1390 | | |
| Week 4 | n | 3 | 3 | 3 | 4 | 4 | 4 |
| | Mean ± SD | 710.0 ± 446.43 | 90.0 ± 181.93 | 11.4 ± 25.76 | 507.5 ± 333.30 | −167.5 ± 191.03 | −19.8 ± 9.58 |
| | Median | 900.0 | −10.0 | −2.2 | 440.0 | −100.0 | −19.3 |
| | Q1, Q3 | 200, 1030 | −20, 300 | −5, 41 | 255, 760 | −300, −35 | −27, −13 |
| | Min, Max | 200, 1030 | −20, 300 | −5, 41 | 200, 950 | −440, −30 | −32, −9 |
| Week 8 | n | 3 | 3 | 3 | 4 | 4 | 4 |
| | Mean ± SD | 666.7 ± 398.29 | 46.7 ± 221.21 | 10.2 ± 27.88 | 875.0 ± 790.55 | 200.0 ± 324.14 | 30.4 ± 31.72 |
| | Median | 760.0 | 20.0 | 9.5 | 535.0 | 120.0 | 38.4 |
| | Q1, Q3 | 230, 1010 | −160, 280 | −17, 38 | 410, 1340 | 0, 400 | 8, 53 |
| | Min, Max | 230, 1010 | −160, 280 | −17, 38 | 380, 2050 | −100, 660 | −14, 58 |
| Week 12 | n | 3 | 3 | 3 | 4 | 4 | 4 |
| | Mean ± SD | 640.0 ± 351.57 | 20.0 ± 36.06 | 6.3 ± 8.24 | 857.5 ± 805.29 | 182.5 ± 329.18 | 23.9 ± 28.49 |
| | Median | 780.0 | 30.0 | 6.8 | 515.0 | 80.0 | 30.3 |
| | Q1, Q3 | 240, 900 | −20, 50 | −2, 14 | 370, 1345 | −20, 385 | 1, 47 |
| | Min, Max | 240, 900 | −20, 50 | −2, 14 | 350, 2050 | −90, 660 | −12, 47 |
| Week 16 | n | 3 | 3 | 3 | 4 | 4 | 4 |
| | Mean ± SD | 736.7 ± 486.76 | 116.7 ± 125.83 | 13.6 ± 13.59 | 505.0 ± 266.15 | −170.0 ± 293.94 | −14.5 ± 19.68 |
| | Median | 830.0 | 100.0 | 13.7 | 505.0 | −30.0 | −5.5 |
| | Q1, Q3 | 210, 1170 | 0, 250 | 0, 27 | 280, 730 | −330, −10 | −25, −4 |
| | Min, Max | 210, 1170 | 0, 250 | 0, 27 | 230, 780 | −610, −10 | −44, −3 |

*CFB = Change from baseline, % CFB = Percent change from baseline.

TABLE 10
Pre bronchodilator Peak Flow [L/min]

| | | Dexpramipexole 150 mg BID | | | | | |
|---|---|---|---|---|---|---|---|
| | | (N = 7) | | | | | |
| | | >=50% AEC reduction at Week 16 | | | <50% AEC reduction at Week 16 | | |
| | | (N = 3) | | | (N = 4) | | |
| Visit | Statistic | Observed | CFB* | % CFB* | Observed | CFB* | % CFB* |
| Screening | n | 3 | | | 4 | | |
| | Mean ± SD | 171.0 ± 39.51 | | | 233.0 ± 138.43 | | |
| | Median | 170.0 | | | 235.5 | | |
| | Q1, Q3 | 132, 211 | | | 148, 319 | | |
| | Min, Max | 132, 211 | | | 61, 400 | | |
| Day 1 | n | 3 | | | 4 | | |
| | Mean ± SD | 160.0 ± 15.72 | | | 156.5 ± 91.58 | | |
| | Median | 157.0 | | | 175.5 | | |
| | Q1, Q3 | 146, 177 | | | 84, 230 | | |
| | Min, Max | 146, 177 | | | 40, 235 | | |
| Baseline | n | 3 | | | 4 | | |
| | Mean ± SD | 160.0 ± 15.72 | | | 156.5 ± 91.58 | | |
| | Median | 157.0 | | | 175.5 | | |
| | Q1, Q3 | 146, 177 | | | 84, 230 | | |
| | Min, Max | 146, 177 | | | 40, 235 | | |
| Week 4 | n | 3 | 3 | 3 | 4 | 4 | 4 |
| | Mean ± SD | 260.7 ± 108.49 | 100.7 ± 108.29 | 63.4 ± 69.42 | 167.5 ± 49.57 | 11.0 ± 107.23 | 90.4 ± 224.72 |
| | Median | 227.0 | 50.0 | 28.2 | 180.5 | −35.5 | −20.4 |
| | Q1, Q3 | 173, 382 | 27, 225 | 18, 143 | 133, 202 | −49, 71 | −24, 205 |
| | Min, Max | 173, 382 | 27, 225 | 18, 143 | 98, 211 | −56, 171 | −25, 428 |
| Week 8 | n | 3 | 3 | 3 | 4 | 4 | 4 |
| | Mean ± SD | 197.7 ± 87.79 | 37.7 ± 93.77 | 25.2 ± 58.36 | 227.0 ± 70.68 | 70.5 ± 97.89 | 136.0 ± 243.51 |
| | Median | 160.0 | 14.0 | 9.6 | 217.5 | 55.5 | 28.5 |
| | Q1, Q3 | 135, 298 | −42, 141 | −24, 90 | 175, 280 | −1, 142 | 4, 268 |
| | Min, Max | 135, 298 | −42, 141 | −24, 90 | 154, 319 | −29, 200 | −13, 500 |
| Week 12 | n | 3 | 3 | 3 | 4 | 4 | 4 |
| | Mean ± SD | 208.7 ± 66.53 | 48.7 ± 72.45 | 31.8 ± 45.54 | 204.8 ± 42.39 | 48.3 ± 98.44 | 121.7 ± 237.65 |
| | Median | 178.0 | 32.0 | 21.9 | 210.5 | 18.0 | 12.2 |
| | Q1, Q3 | 163, 285 | −14, 128 | −8, 82 | 171, 239 | −12, 108 | −5, 249 |
| | Min, Max | 163, 285 | −14, 128 | −8, 82 | 152, 246 | −34, 191 | −15, 478 |
| Week 16 | n | 3 | 3 | 3 | 4 | 4 | 4 |
| | Mean ± SD | 240.0 ± 88.20 | 80.0 ± 87.88 | 50.4 ± 56.32 | 258.0 ± 118.14 | 101.5 ± 180.70 | 236.0 ± 462.72 |
| | Median | 215.0 | 38.0 | 21.5 | 248.0 | 18.5 | 8.1 |
| | Q1, Q3 | 167, 338 | 21, 181 | 14, 115 | 186, 331 | 5, 199 | 1, 471 |
| | Min, Max | 167, 338 | 21, 181 | 14, 115 | 124, 412 | −3, 372 | −2, 930 |

*CFB = Change from baseline, % CFB = Percent change from baseline.

### Post-BD Spirometry

Participants performed post-BD spirometry at specific visits as noted in the SOA.

After completing the pre-BD spirometry, participants inhaled four puffs of albuterol/salbutamol (site sourced) metered dose inhaler (approximately 100 μg albuterol/salbutamol base per actuation), with each actuation 1 minute apart. Post-BD spirometry was assessed within 15 to 30 minutes after inhalation of albuterol/salbutamol.

A summary of FEV₁ values measured after bronchodilation, separated by AECR≥0.5 and AECR<0.5 is provided in Table 11. AECR≥0.5 patients displayed an improvement of 16.6% in FEV₁ after 16 weeks compared to baseline, corresponding to an absolute increase of 220 mL.

TABLE 11
Post bronchodilator Forced Expiratory Volume in 1 Second (FEV₁) [mL]

| | | Dexpramipexole 150 mg BID | | | | | |
|---|---|---|---|---|---|---|---|
| | | (N = 7) | | | | | |
| | | >=50% AEC reduction at Week 16 | | | <50% AEC reduction at Week 16 | | |
| | | (N = 3) | | | (N = 4) | | |
| Visit | Statistic | Observed | CFB* | % CFB* | Observed | CFB* | % CFB* |
| Screening | n | 3 | | | 4 | | |
| | Mean ± SD | 1333.3 ± 479.83 | | | 1347.5 ± 381.52 | | |
| | Median | 1470.0 | | | 1450.0 | | |
| | Q1, Q3 | 800, 1730 | | | 1085, 1610 | | |
| | Min, Max | 800, 1730 | | | 810, 1680 | | |
| Day 1 | n | 3 | | | 4 | | |
| | Mean ± SD | 1373.3 ± 435.01 | | | 1482.5 ± 318.47 | | |
| | Median | 1370.0 | | | 1570.0 | | |
| | Q1, Q3 | 940, 1810 | | | 1270, 1695 | | |
| | Min, Max | 940, 1810 | | | 1030, 1760 | | |
| Baseline | n | 3 | | | 4 | | |
| | Mean ± SD | 1373.3 ± 435.01 | | | 1482.5 ± 318.47 | | |
| | Median | 1370.0 | | | 1570.0 | | |
| | Q1, Q3 | 940, 1810 | | | 1270, 1695 | | |
| | Min, Max | 940, 1810 | | | 1030, 1760 | | |
| Week 16 | n | 3 | 3 | 3 | 4 | 4 | 4 |
| | Mean ± SD | 1593.3 ± 616.95 | 220.0 ± 487.54 | 16.6 ± 35.02 | 1522.5 ± 546.34 | 40.0 ± 260.90 | −0.1± 21.10 |
| | Median | 1700.0 | −10.0 | −1.1 | 1705.0 | 85.0 | 4.8 |
| | Q1, Q3 | 930, 2150 | −110, 780 | −6, 57 | 1170, 1875 | −160, 240 | −15, 15 |
| | Min, Max | 930, 2150 | −110, 780 | −6, 57 | 730, 1950 | −300, 290 | −29, 19 |

*CFB = Change from baseline, % CFB = Percent change from baseline.

Measurements of post-bronchodilation Forced Vital Capacity (FVC), Forced Expiratory Flow 25-75% (FEF₂₅₋₇₅), and Peak Flow are provided in Tables 12, 13, and 14 respectively. These data are separated by AECR≥0.5 and AECR<0.5. Among AECR≥0.5 patients, the average increase in FVC at week 16 compared to baseline was 9.2%, which corresponded to an absolute increase of 226.7 mL. (Table 12). The average FEF₂₅₋₇₅ values for AECR≥0.5 patients increased by 26.7% at week 16 compared to baseline, which corresponds to an absolute increase of 130 mL. (Table 13). The average improvement in Peak Flow for AECR≥0.5 patients was 40.2% at week 16, compared to baseline. (Table 14).

TABLE 12
Post bronchodilator Forced Vital Capacity (FVC) [mL]

| | | Dexpramipexole 150 mg BID | | | | | |
|---|---|---|---|---|---|---|---|
| | | (N = 7) | | | | | |
| | | >=50% AEC reduction at Week 16 | | | <50% AEC reduction at Week 16 | | |
| | | (N = 3) | | | (N = 4) | | |
| Visit | Statistic | Observed | CFB* | % CFB* | Observed | CFB* | % CFB* |
| Screening | n | 3 | | | 4 | | |
| | Mean ± SD | 2546.7 ± 340.34 | | | 2992.5 ± 687.09 | | |
| | Median | 2430.0 | | | 2945.0 | | |
| | Q1, Q3 | 2280, 2930 | | | 2425, 3560 | | |
| | Min, Max | 2280, 2930 | | | 2300, 3780 | | |
| Day 1 | n | 3 | | | 4 | | |
| | Mean ± SD | 2570.0 ± 138.92 | | | 3012.5 ± 407.46 | | |
| | Median | 2500.0 | | | 3130.0 | | |
| | Q1, Q3 | 2480, 2730 | | | 2725, 3300 | | |
| | Min, Max | 2480, 2730 | | | 2440, 3350 | | |
| Baseline | n | 3 | | | 4 | | |
| | Mean ± SD | 2570.0 ± 138.92 | | | 3012.5 ± 407.46 | | |
| | Median | 2500.0 | | | 3130.0 | | |
| | Q1, Q3 | 2480, 2730 | | | 2725, 3300 | | |
| | Min, Max | 2480, 2730 | | | 2440, 3350 | | |
| Week 16 | n | 3 | 3 | 3 | 4 | 4 | 4 |
| | Mean ± SD | 2796.7 ± 527.86 | 226.7 ± 585.26 | 9.2 ± 23.24 | 3082.5 ± 447.84 | 70.0 ± 388.16 | 3.0 ± 14.06 |
| | Median | 2570.0 | −60.0 | −2.4 | 3165.0 | 150.0 | 4.6 |
| | Q1, Q3 | 2420, 3400 | −160, 900 | −6, 36 | 2715, 3450 | −180, 320 | −6, 12 |
| | Min, Max | 2420, 3400 | −160, 900 | −6, 36 | 2540, 3460 | −470, 450 | −16, 18 |

*CFB = Change from baseline, % CFB = Percent change from baseline.

TABLE 13
Post bronchodilator Forced Expiratory Flow 25-75% (FEF₂₅₋₇₅) [mL/s]

| | | Dexpramipexole 150 mg BID | | | | | |
|---|---|---|---|---|---|---|---|
| | | (N = 7) | | | | | |
| | | >=50% AEC reduction at Week 16 | | | <50% AEC reduction at Week 16 | | |
| | | (N = 3) | | | (N = 4) | | |
| Visit | Statistic | Observed | CFB* | % CFB* | Observed | CFB* | % CFB* |
| Screening | n | 3 | | | 4 | | |
| | Mean ± SD | 593.3 ± 345.30 | | | 1052.5 ± 1074.78 | | |
| | Median | 690.0 | | | 685.0 | | |
| | Q1, Q3 | 210, 880 | | | 330, 1775 | | |
| | Min, Max | 210, 880 | | | 240, 2600 | | |
| Day 1 | n | 3 | | | 4 | | |
| | Mean ± SD | 680.0 ± 461.30 | | | 560.0 ± 192.70 | | |
| | Median | 640.0 | | | 545.0 | | |
| | Q1, Q3 | 240, 1160 | | | 440, 680 | | |
| | Min, Max | 240, 1160 | | | 340, 810 | | |
| Baseline | n | 3 | | | 4 | | |
| | Mean ± SD | 680.0 ± 461.30 | | | 560.0 ± 192.70 | | |
| | Median | 640.0 | | | 545.0 | | |
| | Q1, Q3 | 240, 1160 | | | 440, 680 | | |
| | Min, Max | 240, 1160 | | | 340, 810 | | |
| Week 16 | n | 3 | 3 | 3 | 4 | 4 | 4 |
| | Mean ± SD | 810.0 ± 474.45 | 130.0 ± 350.86 | 26.7 ± 49.08 | 650.0 ± 382.19 | 90.0 ± 225.24 | 9.7 ± 40.69 |
| | Median | 1000.0 | 30.0 | 12.5 | 635.0 | 90.0 | 7.3 |
| | Q1, Q3 | 270, 1160 | −160, 520 | −14, 81 | 340, 960 | −105, 285 | −25, 44 |
| | Min, Max | 270, 1160 | −160, 520 | −14, 81 | 240, 1090 | −110, 290 | −29, 54 |

*CFB = Change from baseline, % CFB = Percent change from baseline.

TABLE 14
Post bronchodilator Peak Flow [L/min]

| | | Dexpramipexole 150 mg BID | | | | | |
|---|---|---|---|---|---|---|---|
| | | (N = 7) | | | | | |
| | | >=50% AEC reduction at Week 16 | | | <50% AEC reduction at Week 16 | | |
| | | (N = 3) | | | (N = 4) | | |
| Visit | Statistic | Observed | CFB* | % CFB* | Observed | CFB* | % CFB* |
| Screening | n | 3 | | | 4 | | |
| | Mean ± SD | 179.0 ± 46.81 | | | 246.5 ± 113.58 | | |
| | Median | 154.0 | | | 243.0 | | |
| | Q1, Q3 | 150, 233 | | | 176, 317 | | |
| | Min, Max | 150, 233 | | | 111, 389 | | |
| Day 1 | n | 3 | | | 4 | | |
| | Mean ± SD | 168.7 ± 10.41 | | | 274.5 ± 95.94 | | |
| | Median | 172.0 | | | 292.5 | | |
| | Q1, Q3 | 157, 177 | | | 198, 352 | | |
| | Min, Max | 157, 177 | | | 154, 359 | | |
| Baseline | n | 3 | | | 4 | | |
| | Mean ± SD | 168.7 ± 10.41 | | | 274.5 ± 95.94 | | |
| | Median | 172.0 | | | 292.5 | | |
| | Q1, Q3 | 157, 177 | | | 198, 352 | | |
| | Min, Max | 157, 177 | | | 154, 359 | | |
| Week 16 | n | 3 | 3 | 3 | 4 | 4 | 4 |
| | Mean ± SD | 233.0 ± 77.58 | 64.3 ± 86.56 | 40.2 ± 55.44 | 326.0 ± 148.82 | 51.5 ± 88.04 | 16.9 ± 28.64 |
| | Median | 215.0 | 38.0 | 21.5 | 324.5 | 46.0 | 18.1 |
| | Q1, Q3 | 166, 318 | −6, 161 | −3, 103 | 227, 425 | −22, 125 | −8, 42 |
| | Min, Max | 166, 318 | −6, 161 | −3, 103 | 146, 509 | −36, 150 | −10, 42 |

*CFB = Change from baseline, % CFB = Percent change from baseline.

### Computed Tomography (CT)

All eligible participants received inspiratory and expiratory CT scans at Baseline and at Week 16 to assess for changes in mucus plugs and airway architecture (e.g., changes in airway wall thickness).

A total of completed week 16 underwent at CT scan with mucus plug scoring. The mucus plug score for all 6 patients is provide in Table 15. One patient reduced from a mucus plug score of 4 at baseline to a score of 0 at week 16.

TABLE 15
CT Scan - Mucus Score

| | | | | Change from |
|---|---|---|---|---|
| Subject ID | Visit | Date/Time | Result | Baseline |
| 10001-001- | Day 1* | 2024 Aug. 6/10:29 | 1.00 | |
| 0001 | Week 16 | 2024 Nov. 26/11:18 | 1.00 | 0.00 |
| 10001-001- | Day 1* | 2024 Aug. 5/11:01 | 0.00 | |
| 0002 | Week 16 | 2024 Dec. 2/13:35 | 0.00 | 0.00 |
| 10001-008- | Day 1* | 2024 Sep. 18/17:22 | 0.00 | |
| 0002 | Week 16 | 2025 Jan. 27/14:55 | 3.00 | 3.00 |
| 10001-010- | Day 1* | 2024 Dec. 10/11:39 | 0.00 | |
| 0005 | Week 16 | 2025 Mar. 19/11:07 | 0.00 | 0.00 |
| 10001-022- | Day 1* | 2024 Nov. 6/12:07 | 4.00 | |
| 0001 | Week 16 | 2025 Mar. 5/13:09 | 0.00 | −4.00 |
| 10001-022- | Day 1* | 2024 Nov. 11/10:07 | 3.00 | |
| 0006 | Week 16 | 2025 Mar. 12/12:05 | 6.00 | 3.00 |

*Is derived as a Baseline result prior to first dose of study treatment.

The results of CT scoring for emphysema are displayed in Table 16. One patient displayed a decrease of 4.75 points.

TABLE 16
CT Scan Parametric Imaging - Emphysema

| | | | | Change from |
|---|---|---|---|---|
| Subject ID | Visit | Date/Time | Result | Baseline |
| 10001-001- | Day 1* | 2024 Aug. 6/10:29 | 33.87 | |
| 0001 | Week 16 | 2024 Nov. 26/11:18 | 36.28 | 2.41 |
| 10001-008- | Day 1* | 2024 Sep. 18/17:22 | 36.60 | |
| 0002 | Week 16 | 2025 Jan. 27/14:55 | 35.88 | −0.73 |
| 10001-010- | Day 1* | 2024 Dec. 10/11:39 | 11.95 | |
| 0005 | Week 16 | 2025 Mar. 19/11:07 | 12.11 | 0.16 |
| 10001-022- | Day 1* | 2024 Nov. 6/12:07 | 13.34 | |
| 0001 | Week 16 | 2025 Mar. 5/13:09 | 8.59 | −4.75 |

*Is derived as a Baseline result prior to first dose of study treatment.

The results of CT scoring for small airway disease are displayed in Table 17. All patients showed an improvement in scoring, with one patient improving by 7.12 points.

TABLE 17
CT Scan - Small Airways Disease

| | | | | Change from |
|---|---|---|---|---|
| Subject ID | Visit | Date/Time | Result | Baseline |
| 10001-001- | Day 1* | 2024 Aug. 6/10:29 | 32.83 | |
| 0001 | Week 16 | 2024 Nov. 26/11:18 | 25.72 | −7.12 |
| 10001-008- | Day 1* | 2024 Sep. 18/17:22 | 48.96 | |
| 0002 | Week 16 | 2025 Jan. 27/14:55 | 51.14 | 2.18 |
| 10001-010- | Day 1* | 2024 Dec. 10/11:39 | 42.88 | |
| 0005 | Week 16 | 2025 Mar. 19/11:07 | 39.08 | −3.80 |
| 10001-022- | Day 1* | 2024 Nov. 6/12:07 | 50.48 | |
| 0001 | Week 16 | 2025 Mar. 5/13:09 | 46.36 | −4.12 |

The results of CT scoring for Air Trapping are displayed in Table 18. One patient displayed a change of −11.40.

TABLE 18
CT Scan - Air Trapping Index -856 HU [%]

| | | | Inspiratory | | Expiratory | |
|---|---|---|---|---|---|---|
| | | | | Change from | | Change from |
| Subject ID | Visit | Date/Time | Result | Baseline | Result | Baseline |

| | | | | |
|---|---|---|---|---|
| 10001-001-0001 | Day 1* | 2024 Aug. 6/10:32 | 49.86 | |
| | Week 16 | 2024 Nov. 26/11:19 | 49.57 | −0.29 |
| 10001-008-0002 | Day 1* | 2024 Sep. 18/17:24 | 67.49 | |
| | Week 16 | 2025 Jan. 27/14:56 | 68.06 | 0.57 |
| 10001-010-0005 | Day 1* | 2024 Dec. 10/11:45 | 31.75 | |
| | Week 16 | 2025 Mar. 19/11:07 | 29.57 | −2.18 |
| 10001-022-0001 | Day 1* | 2024 Nov. 6/12:10 | 39.28 | |
| | Week 16 | 2025 Mar. 5/13:12 | 27.88 | −11.40 |

### Moderate or Severe COPD Exacerbations

Moderate or severe COPD exacerbations were documented for descriptive purposes but not formally analyzed.

A moderate COPD exacerbation was defined as an acute exacerbation of COPD that requires either systemic corticosteroids (intramuscular [IM], intravenous, or oral) and/or antibiotics.

A severe exacerbations was defined as an acute exacerbation of COPD that requires hospitalization (defined as inpatient admission≥24 hours in hospital, observation area, emergency department, or other equivalent health care facility, depending on the health care system) or results in death.

If less than 7 days have elapsed since the end date of a COPD exacerbation and the start date of a new COPD exacerbation, the second event was considered a relapse of the prior COPD exacerbation.

The start of an exacerbation was defined as the start date of systemic corticosteroids and/or antibiotics, date of emergency department (ED) or urgent care visits, or date of hospital admission due to COPD, whichever occurs earlier. The end date of an exacerbation was defined as the last date of systemic corticosteroids and/or antibiotics, date of ED or urgent care visit, or date of hospital discharge, whichever occurs later.

A COPD exacerbation that occurred during the treatment period is treated according to standard practice.

### Participant Reported Outcomes

### St George's Respiratory Questionnaire for COPD Patients (SGRQ-C)

The SGRQ-C questionnaire was developed from the SGRQ which was designed to measure health impairment in patients with asthma and COPD. The SGRQ-C is a shorter version (40 items compared with 50 items in SGRQ) derived from the original version following detailed analysis of data from large studies in COPD. The FDA has stated that the SGRQ-C is acceptable for use as either a co-primary endpoint or as a secondary endpoint providing supporting evidence of efficacy in a clinical trial. In this study, assessment of SGRQ-C was a secondary endpoint.

The SGRQ-C total score is made up of three components: (1) symptoms frequency and severity of symptoms; (2) activity effect of disease on common daily physical activities; and (3) impacts psychosocial effects of the disease.

The threshold for a clinically significant difference between groups of participants and for changes within groups of participants was four units.

Table 19 shows the result of the SGRQ-C questionnaire for patients that completed week 16, separated by their status as AECR≥0.5 or AECR≥0.5. AECR≥0.5 patients displayed a clinically significant decrease of 12.08 points at 16 weeks compared to baseline.

TABLE 19
SGRQ-C Questionnaire - Total Score

| | | Dexpramipexole 150 mg BID | | | |
|---|---|---|---|---|---|
| | | (N = 7) | | | |
| | | >=50% AEC reduction at | | <50% AEC reduction at | |
| | | Week 16 (N = 3) | | Week 16 (N = 4) | |
| Visit | Statistic | Observed | CFB* | Observed | CFB* |
| Baseline | n | 3 | | 4 | |
| | Mean ± SD | 52.47 ± 12.431 | | 49.60 ± 21.140 | |
| | Median | 57.81 | | 58.17 | |
| | Q1, Q3 | 38.3, 61.3 | | 37.6, 61.6 | |
| | Min, Max | 38.3, 61.3 | | 18.2, 63.9 | |
| Week 16 | n | 3 | 3 | 4 | 4 |
| | Mean ± SD | 40.39 ± 7.250 | −12.08 ± 5.544 | 45.45 ± 19.893 | −4.15 ± 6.817 |
| | Median | 44.09 | −12.76 | 50.64 | −5.18 |
| | Q1, Q3 | 32.0, 45.0 | −17.2, −6.2 | 32.1, 58.8 | −9.8, 1.5 |
| | Min, Max | 32.0, 45.0 | −17.2, −6.2 | 17.3, 63.2 | −10.1, 3.9 |

*CFB = Change from baseline

### COPD Assessment Test (CAT)

The CAT is a participant-completed instrument that can quantify the impact of COPD on the participant's health. It complements existing approaches to assessing COPD, such as FEV₁ measurement. It was initially designed, using a rigorous scientific development process, to provide a simple and reliable measure of health status in COPD to aid assessment of participants and promote communication between participants and clinicians. Researchers have recommended that the CAT be used as a US FDA assessment tool.

The CAT is a validated, short (8-item) and simple participant-completed questionnaire, with good discriminant properties, developed for use in routine clinical practice to measure the health status of participants with COPD. Despite the small number of component items, it covers a broad range of effects of COPD on participants' health. Studies have shown that it is responsive to change and to treatment.

The CAT has a scoring range of 0-40 (0 to 5 on 8 questions) with higher numbers indicating more severe symptoms. A difference or change of two or more units over 2 to 3 months in a participant suggested a clinically significant difference or change in health status.

Table 20 shows the result of the CAT for patients that completed week 16, separated by their status as AECR≥0.5 or AECR<0.5. AECR≥0.5 patients displayed a clinically significant decrease of 8.7 points at week 16 compared to baseline.

TABLE 20
COPD Assessment - Total Score

| | | Dexpramipexole 150 mg BID | | | |
|---|---|---|---|---|---|
| | | (N = 7) | | | |
| | | >=50% AEC reduction at | | <50% AEC reduction at | |
| | | Week 16 (N = 3) | | Week 16 (N = 4) | |
| Visit | Statistic | Observed | CFB* | Observed | CFB* |
| Baseline | n | 3 | | 4 | |
| | Mean ± SD | 25.0 ± 5.57 | | 19.3 ± 7.09 | |
| | Median | 24.0 | | 22.0 | |
| | Q1, Q3 | 20, 31 | | 15, 24 | |
| | Min, Max | 20, 31 | | 9, 24 | |
| Week 16 | n | 3 | 3 | 4 | 4 |
| | Mean ± SD | 16.3 ± 5.86 | −8.7 ± 1.15 | 17.5 ± 8.27 | −1.8 ± 1.50 |
| | Median | 14.0 | −8.0 | 20.0 | −2.0 |
| | Q1, Q3 | 12, 23 | −10, −8 | 12, 24 | −3, −1 |
| | Min, Max | 12, 23 | −10, −8 | 6, 24 | −3, 0 |

*CFB = Change from baseline

### Assessment of Pharmacokinetics

Plasma samples from each participant were drawn to measure drug concentrations as per the SOA. Samples were drawn prior to dosing with investigational drug.

### Assessment of Biomarkers and Transcriptomics

Samples for inflammatory biomarkers from blood including interleukin (IL)1, IL-13, IL-4, IL-5, IL-13, IL-6, IL-8, TNFα superfamily, fibrinogen, eosinophil-derived neurotoxin, eosinophil peroxidase, eosinophil cationic protein were collected at Baseline Visit and Week 16. Samples for blood and nasal transcriptomics were collected predose at Baseline and at Week 16.

The phraseology or terminology herein is for the purpose of description and not of limitation. As such, the terminology and/or phraseology of the present specification should be interpreted by the skilled artisan in light of the teachings and guidance herein.

The breadth and scope of the present invention should not be limited by any of the above-described exemplary embodiments, but should be defined only in accordance with the following claims and their equivalents.

All patents, patent applications, and other references noted or referenced in this application are hereby incorporated by reference in their entirety.

1. A method of reducing the rate of COPD exacerbations in a human subject with eosinophilic chronic obstructive pulmonary disorder (COPD), comprising orally administering to the subject a therapeutically effective amount of dexpramipexole, or a pharmaceutically acceptable salt thereof.

2. The method of claim 1, wherein dexpramipexole, or a pharmaceutically acceptable salt thereof, is administered in a daily dose of about 150 mg to about 300 mg per day.

3.-10. (canceled)

11. The method of claim 1, wherein the subject has evidence of mucus plugs (i.e., a mucus plug score of ≥1) before administration of dexpramipexole, or a pharmaceutically acceptable salt thereof.

12. The method of claim 11, wherein administering dexpramipexole, or a pharmaceutically acceptable salt thereof, reduces the mucus plug score.

13. The method of claim 1, wherein administering dexpramipexole, or a pharmaceutically acceptable salt thereof, improves the subject's mean forced expiratory volume in 1 second (FEV₁).

14.-21. (canceled)

22. The method of claim 1, wherein administering dexpramipexole, or a pharmaceutically acceptable salt thereof, improves the subject's forced vital capacity (FVC).

23.-33. (canceled)

34. The method of claim 1, wherein administering dexpramipexole, or a pharmaceutically acceptable salt thereof, improves the subject's forced mid-expiratory flow (FEF₂₅₋₇₅).

35. The method of claim 1, wherein administering dexpramipexole, or a pharmaceutically acceptable salt thereof, improves the subject's lung function.

36. The method of claim 1, wherein administering dexpramipexole, or a pharmaceutically acceptable salt thereof, improves a measurement selected from the group consisting of St George's Respiratory Questionnaire for COPD Patients (SGRQ-C), COPD Assessment Test (CAT), Modified Medical Research Council Grading System (mMRC), Evaluating Respiratory Symptoms in Chronic Obstructive Pulmonary Disease (E-RS:COPD), and any combination thereof.

37. The method of claim 1, wherein prior to treatment with dexpramipexole or a pharmaceutically acceptable salt thereof, the subject is already receiving a treatment regimen comprising administration of an inhaled corticosteroid (ICS), thereby treating the COPD in the subject.

38. The method of claim 37, wherein the treatment regimen comprises administration of an ICS and a long-acting β2 agonist (LABA).

39.-48. (canceled)

49. A method of reducing the presence of mucus plugs as demonstrated by reducing a mucus plug score in a human subject with eosinophilic COPD, comprising orally administering to the subject a therapeutically effective amount of dexpramipexole, or a pharmaceutically acceptable salt thereof.

50. The method of claim 49, wherein dexpramipexole, or a pharmaceutically acceptable salt thereof, is administered in a daily dose of about 150 mg to about 300 mg per day.

51.-60. (canceled)

61. The method of claim 49, wherein administering dexpramipexole, or a pharmaceutically acceptable salt thereof, improves the subject's mean forced expiratory volume in 1 second (FEV₁).

62.-69. (canceled)

70. The method of claim 49, wherein administering dexpramipexole, or a pharmaceutically acceptable salt thereof, improves the subject's forced vital capacity (FVC).

71.-81. (canceled)

82. The method of claim 49, wherein administering dexpramipexole, or a pharmaceutically acceptable salt thereof, improves the subject's forced mid-expiratory flow (FEF25-75).

83. The method of claim 49, wherein administering dexpramipexole, or a pharmaceutically acceptable salt thereof, improves the subject's lung function.

84. The method of claim 49, wherein administering dexpramipexole, or a pharmaceutically acceptable salt thereof, improves a measurement selected from the group consisting of St George's Respiratory Questionnaire for COPD Patients (SGRQ-C), COPD Assessment Test (CAT), Modified Medical Research Council Grading System (mMRC), Evaluating Respiratory Symptoms in Chronic Obstructive Pulmonary Disease (E-RS:COPD), and any combination thereof.

85. The method of claim 49, wherein prior to treatment with dexpramipexole or a pharmaceutically acceptable salt thereof, the subject is already receiving a treatment regimen comprising administration of an inhaled corticosteroid (ICS), thereby treating the COPD in the subject.

86. The method of claim 85, wherein the treatment regimen comprises administration of an ICS and a long-acting β2 agonist (LABA).

87.-96. (canceled)

97. A method of reducing rate of hospitalization due to exacerbations in a human subject with eosinophilic COPD, comprising orally administering to the subject a therapeutically effective amount of dexpramipexole, or a pharmaceutically acceptable salt thereof.

98. The method of claim 97, wherein dexpramipexole, or a pharmaceutically acceptable salt thereof, is administered in a daily dose of about 150 mg to about 300 mg per day.

99.-132. (canceled)

133. The method of claim 97, wherein prior to treatment with dexpramipexole or a pharmaceutically acceptable salt thereof, the subject is already receiving a treatment regimen comprising administration of an inhaled corticosteroid (ICS), thereby treating the COPD in the subject.

134. The method of claim 133, wherein the treatment regimen comprises administration of an ICS and a long-acting β2 agonist (LABA).

135.-144. (canceled)

145. A method of reducing the rate of oral/parenteral steroid and/or antibiotic use for treatment of COPD in a human subject with eosinophilic COPD, comprising orally administering to the subject a therapeutically effective amount of dexpramipexole, or a pharmaceutically acceptable salt thereof.

146. The method of claim 145, wherein dexpramipexole, or a pharmaceutically acceptable salt thereof, is administered in a daily dose of about 150 mg to about 300 mg per day.

147.-156. (canceled)

157. The method of claim 145, wherein administering dexpramipexole, or a pharmaceutically acceptable salt thereof, improves the subject's mean forced expiratory volume in 1 second (FEV₁).

158.-165. (canceled)

166. The method of claim 145, wherein administering dexpramipexole, or a pharmaceutically acceptable salt thereof, improves the subject's forced vital capacity (FVC).

167.-180. (canceled)

181. The method of claim 145, wherein prior to treatment with dexpramipexole or a pharmaceutically acceptable salt thereof, the subject is already receiving a treatment regimen comprising administration of an inhaled corticosteroid (ICS), thereby treating the COPD in the subject.

182. The method of claim 181, wherein the treatment regimen comprises administration of an ICS and a long-acting β2 agonist (LABA).

183.-240. (canceled)
